(19) United States
(12) Patent Application Publication
(10) Pub. No.: US 2026/0217828 A1
(43) Pub. Date: Jul. 30, 2026

(54) IMMUNE CHECKPOINT BLOCKADE THERAPY FOR TREATING STAPHYLOCOCCUS AUREUS INFECTIONS

(71) Applicant: University of Rochester, Rochester, NY (US)

(72) Inventors: Gowrishankar Muthukrishnan, Rochester, NY (US); Edward Schwarz, Rochester, NY (US); Motoo M. Saito, Rochester, NY (US)

(73) Assignee: University of Rochester, Rochester, NY (US)

(21) Appl. No.: 19/148,891

(22) PCT Filed: Jan. 30, 2024
(86) PCT No.: PCT/US24/13553

§ 371 Date: Jul. 17, 2025

Related U.S. Application Data
(60) Provisional application No. 63/482,322, filed on Jan. 31, 2023.
(60) Provisional application No. 63/509,689, filed on Jun. 22, 2023.

(51) Int. Cl. C07K 16/28; A61K 39/395; A61K 45/06; A61P 31/04; G01N 33/68

(52) U.S. Cl. CPC C07K 16/2818; A61K 39/3955; A61K 45/06; A61P 31/04; C07K 16/2803; G01N 33/6893; C07K 2317/31; G01N 2333/521; G01N 2333/54; G01N 2333/70503; G01N 2333/70521; G01N 2800/26; G01N 2800/52

(57) ABSTRACT

This disclosure provides a method reducing the number of Staphylococcus aureus bacterial cells in a subject and a method of treating or preventing diseases or disorders caused by S. aureus. This disclosure also provides a method of diagnosis or prognosis of a disease or disorder caused by S. aureus.

### CROSS REFERENCE TO RELATED APPLICATIONS

This application claims priority to U.S. Provisional Application No. 63/482,322 filed on Jan. 31, 2023 and U.S. Provisional Application No. 63/509,689 filed on Jun. 22, 2023. The content of the applications are incorporated herein by reference in their entireties.

### STATEMENT REGARDING FEDERALLY SPONSORED RESEARCH

This invention was made with government support under AI169736 awarded by the National Institutes of Health. The government has certain rights in the invention.

### FIELD OF THE INVENTION

This invention relates generally to an immune checkpoint therapy for treating or preventing Staphylococcus aureus infections.

### BACKGROUND OF THE INVENTION

Staphylococcus aureus, a gram-positive coccal bacterium and a member of the Firmicutes, is frequently found in the nose, respiratory tract, and on skin. It is a common cause of skin infections, respiratory infections such as sinusitis, and food poisoning. Pathogenic strains often promote infections by producing potent protein toxins, and expressing cell-surface proteins that bind and inactivate antibodies. The emergence of antibiotic-resistant strains of S. aureus, such as methicillin-resistant S. aureus (MRSA), is a global problem in clinical medicine. It is a substantial cause of sickness and death in both humans and animals. Infection with S. aureus often results in the development of a superficial abscess. Other cases of S. aureus infection can be much more serious. For example, intrusion of S. aureus into the lymphatics and blood can lead to a systemic infection, which in turn can cause complications such as endocarditis, arthritis, osteomyelitis, pneumonia, septic shock, and even death. Hospital-acquired S. aureus infection is common and particularly problematic, with S. aureus being the most frequent cause of hospital-acquired surgical site infections and pneumonia and the second most frequent cause of cardiovascular and bloodstream infections. S. aureus is also the most common pathogen in orthopedic surgical site infections, which leads to sepsis and death in 10% of patients (Cram P, et al. JAMA 308-12, 1227-1236 (2012). Antibiotic administration has been the standard treatment for S. aureus infections. Unfortunately, the use of antibiotics has also fueled the development of antibiotic resistance in S. aureus.

Although vaccines against S. aureus are being developed and numerous trials based on production of antibodies have been performed, all of them have failed. Most notably, a large phase 2 clinical trial of an iron-regulated surface determinants protein B (IsdB) active vaccine had to be terminated early due to sepsis and multi-organ failure after the surgery (Fowler V G, et al. JAMA 2013; 309:1368-78), and increased mortality has been observed in patients with S. aureus prosthetic joint infections (PJI) that have high anti-IsdB antibody titers (Nishitani K, et al. Clin Orthop Relat Res 473-9, 2735-49 (2015).

Therefore, there is a need for improved methods for treating and preventing S. aureus infections and related disorders.

### SUMMARY OF INVENTION

This disclosure addresses the need mentioned above in a number of aspects.

In one aspect, this disclosure provides a method for reducing the number of cells of a pathogenic bacterium or a pathogenic fungus, such as S. aureus bacterial cells, in a subject in need thereof. In some embodiments, the method comprises administering to the subject an effective amount of an inhibitor of an immune checkpoint molecule.

In some embodiments, the immune checkpoint molecule is selected from the group consisting of LAG-3, TIM-3, CTLA-4, PD-1, and PD-L1.

In some embodiments, the inhibitor comprises an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof.

In some embodiments, the inhibitor comprises an anti-LAG-3 antibody, an anti-TIM-3 antibody, an anti-CTLA-4 antibody, an anti-PD-1 antibody, an anti-PD-L1 antibody, or a combination thereof. In some embodiments, the inhibitor comprises (i) an anti-LAG-3 antibody or an antigen-binding fragment thereof, (ii) a combination of an anti-PD-1 antibody or an antigen-binding fragment thereof and an anti-LAG-3 antibody or an antigen-binding fragment thereof, or (iii) a bispecific antibody that binds PD-1 and LAG-3. In some embodiments, the anti-PD-1 antibody is nivolumab, or the anti-LAG-3 antibody is relatlimab.

In another aspect, this disclosure also provides a method of diagnosis or prognosis of a disease or disorder caused by a pathogenic bacterium or a pathogenic fungus, such as S. aureus, in a subject in need thereof. In some embodiments, the method comprises: (a) determining a level of each of a set of biomarkers in a sample from the subject, wherein the set of biomarkers comprises an immune checkpoint molecule or a cytokine; (b) determining a change in the level of each of the set of biomarkers as compared to a reference level for each of the set of biomarkers; and (c) assessing the presence of the disease or disorder or a status of the disease or disorder based on the change in the level of each of the set of biomarkers as compared to the reference level for each of the set of biomarkers.

In some embodiments, the set of biomarkers comprises one or more of LAG-3, PD-1, CTLA-4, TIM-3, IFNγ, IL-2, TNFα, and IL-17.

In some embodiments, the set of biomarkers comprises TIM-3. In some embodiments, the set of biomarkers comprises TIM-3 and LAG-3. In some embodiments, the set of biomarkers comprises TIM-3 and CTLA-4. In some embodiments, the set of biomarkers comprises TIM-3 and PD-1. In some embodiments, the set of biomarkers comprises TIM-3, LAG-3, and PD-1. In some embodiments, the set of biomarkers comprises TIM-3, LAG-3, and CTLA-4. In some embodiments, the set of biomarkers comprises TIM-3, CTLA-4, and PD-1. In some embodiments, the set of biomarkers comprises TIM-3, LAG-3, CTLA-4, and PD-1. In some embodiments, the set of biomarkers comprises TIM-3, LAG-3, and PD-1. In some embodiments, the set of biomarkers comprises TIM-3, LAG-3, and CTLA-4. In some embodiments, the set of biomarkers comprises TIM-3, CTLA-4, and PD-1. In some embodiments, the set of biomarkers comprises TIM-3, LAG-3, CTLA-4, and PD-1.

In some embodiments, the set of biomarkers comprises TIM-3 and IL-17. In some embodiments, the set of biomarkers comprises TIM-3, LAG-3, and IL-17. In some embodiments, the set of biomarkers comprises TIM-3, CTLA-4, and IL-17. In some embodiments, the set of biomarkers comprises TIM-3, PD-1, and IL-17. In some embodiments, the set of biomarkers comprises TIM-3, LAG-3, PD-1, and IL-17. In some embodiments, the set of biomarkers comprises TIM-3, LAG-3, CTLA-4, and IL-17. In some embodiments, the set of biomarkers comprises TIM-3, CTLA-4, PD-1, and IL-17. In some embodiments, the set of biomarkers comprises TIM-3, LAG-3, CTLA-4, PD-1, and IL-17.

In some embodiments, the change in the level of each of the set of biomarkers is an increase of an expression level of each of the set of biomarkers.

In yet another aspect, this disclosure further provides a method of preventing, treating or ameliorating a disease or disorder caused by a pathogenic bacterium or a pathogenic fungus (such as S. aureus) in a subject in need thereof. In some embodiments, the method comprises administering to the subject a therapeutically effective amount of an inhibitor of an immune checkpoint molecule.

In some embodiments, the method further comprises selecting a subject having a chronic or acute disease or disorder caused by the pathogenic bacterium or a pathogenic fungus according to the method described herein.

In some embodiments, the disease or disorder comprises an infection, e.g., an infection of S. aureus. In some embodiments, the infection is bacteremia. In some embodiments, the infection is a bone infection. In some embodiments, the infection is osteomyelitis. In some embodiments, the disease or disorder comprises S. aureus-associated sepsis.

In some embodiments, the infection is a prosthetic joint infection, a fracture related infection, a diabetic foot infection, a hematogenous osteomyelitis, or a spine infection.

In some embodiments, the immune checkpoint molecule is selected from the group consisting of LAG-3, TIM-3, CTLA-4, PD-1, and PD-L1.

In some embodiments, the inhibitor comprises an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof.

In some embodiments, the inhibitor comprises an anti-LAG-3 antibody, an anti-TIM-3 antibody, an anti-CTLA-4 antibody, an anti-PD-1 antibody, an anti-PD-L1 antibody, an antigen-binding fragment thereof, or a combination thereof. In some embodiments, the inhibitor comprises (i) an anti-LAG-3 antibody or an antigen-binding fragment thereof, (ii) a combination of an anti-PD-1 antibody or an antigen-binding fragment thereof and an anti-LAG-3 antibody or an antigen-binding fragment thereof, or (iii) a bispecific antibody that binds PD-1 and LAG-3. In some embodiments, the anti-PD-1 antibody is nivolumab, or the anti-LAG-3 antibody is relatlimab.

In some embodiments, the method comprises administering to the subject an additional therapeutic agent. In some embodiments, the additional therapeutic agent comprises a second inhibitor of a second immune checkpoint molecule, an antibiotic, an antipathogen antibody specific for the pathogenic bacterium or pathogenic fungus, or a combination thereof. In some embodiments the antipathogen antibody specifically binds to Staphylococcus aureus. In some embodiments, the inhibitor and the additional therapeutic agent are contained in the same composition.

In some embodiments, the second immune checkpoint molecule is selected from the group consisting of LAG-3, TIM-3, CTLA-4, PD-1, and PD-L1.

In some embodiments, the second inhibitor comprises an anti-LAG-3 antibody, an anti-TIM-3 antibody, an anti-CTLA-4 antibody, an anti-PD-1 antibody, an anti-PD-L1 antibody, an antigen-binding fragment thereof, or a combination thereof.

In some embodiments, the antibiotic has an anti-bacterial activity against S. aureus.

In some embodiments, the additional therapeutic agent is administered concurrently with the inhibitor. In some embodiments, the additional therapeutic agent is administered before or after the inhibitor.

In some embodiments, the inhibitor or the additional therapeutic agent is administered to the subject intratumorally, intravenously, subcutaneously, intraosseously, orally, transdermally, in sustained release, in controlled release, in delayed release, as a suppository, or sublingually.

In some embodiments, the subject has received or is about to receive a surgery or an implant. In some embodiments the subject received a surgery or an implant within 1 week, 2 weeks, 1 month, 3 months, 6 months, 9 months, 12 months, 18 months, or 24 months. In some embodiments the subject will receive surgery or an implant within 1 week, 2 weeks, 1 month, 3 months, 6 months, 9 months, 12 months, 18 months, or 24 months.

In some embodiments, the surgery is selected from the group consisting of orthopedic surgery, cardiothoracic surgery, plastic surgery, neurosurgery, oral surgery, total joint replacement, open reduction internal fixation (ORIF), debridement for open fracture, spine surgery, median sternotomy, revision total joint, revision ORIF, drainage of soft tissue abscess, or organ transplantation surgery.

In some embodiments, the disclosure provides:
[1] a method for reducing the number of cells of a pathogenic bacterium or a pathogenic fungus in a subject in need thereof, comprising administering to the subject an effective amount of an inhibitor of an inhibitory immune checkpoint molecule;
[2] the method of [1], wherein the administered inhibitor inhibits an inhibitory immune checkpoint molecule selected from the group consisting of LAG-3, TIM-3, CTLA-4, PD-1, and PD-L1;
[3] the method of [1] or [2], wherein the administered inhibitor comprises an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof;
[4] the method of any one of [1]-[3], wherein the administered inhibitor comprises an anti-LAG-3 antibody, an anti-TIM-3 antibody, an anti-CTLA-4 antibody, an anti-PD-1 antibody, an anti-PD-L1 antibody, an antigen-binding fragment thereof, or a combination thereof;
[5] the method of any one of [1]-[4], wherein the administered inhibitor comprises (i) an anti-LAG-3 antibody or an antigen-binding fragment thereof, (ii) a combination of an anti-PD-1 antibody or an antigen-binding fragment thereof and an anti-LAG-3 antibody or an antigen-binding fragment thereof, or (iii) a bispecific antibody that binds PD-1 and LAG-3;
[6] the method of [5], wherein the administered anti-PD-1 antibody is nivolumab, the anti-LAG-3 antibody is relatlimab, or the bispecific antibody comprises an antigen binding fragment of nivolumab and an antigen binding fragment of relatlimab;
[7] a method of diagnosis or prognosis of a disease or disorder caused by a pathogenic bacterium or a pathogenic fungus in a subject in need thereof, comprising:
determining a level of each of a set of biomarkers in a sample from the subject], wherein the set of biomarkers comprises an inhibitory immune checkpoint molecule or a cytokine;
determining a change in the level of each of the set of biomarkers as compared to a reference level for each of the set of biomarkers; and
assessing the presence of the disease or disorder or a status of the disease or disorder based on the change in the level of each of the set of biomarkers as compared to the reference level for each of the set of biomarkers;
[8] the method of [7], wherein the set of biomarkers comprises one or more of
   (a) LAG-3, PD-1, CTLA-4, TIM-3, IFNγ, IL-2, TNFα, and IL-17; or
   (b) LAG-3, TIM-3, and CXCL13;
[9] the method of [7] or [8]], wherein the set of biomarkers comprises: (a) TIM-3; (b) TIM-3 and LAG-3; (c) TIM-3 and CTLA-4; (d) TIM-3 and PD-1; (e) TIM-3 and IL-17; (f) TIM-3, LAG-3, and PD-1; (g) TIM-3, LAG-3, and CTLA-4; (h) TIM-3, CTLA-4, and PD-1; (i) TIM-3, LAG-3, CTLA-4, and PD-1; (j) TIM-3, LAG-3, and IL-17; (k) TIM-3, CTLA-4, and IL-17; (1) TIM-3, PD-1, and IL-17; (m) TIM-3, LAG-3, PD-1, and IL-17; (n) TIM-3, LAG-3, CTLA-4, and IL-17; (o) TIM-3, CTLA-4, PD-1, and IL-17; or (p) TIM-3, LAG-3, CTLA-4, PD-1, and IL-17;
[10] the method of any one of [7]-[9], wherein the set of biomarkers comprises TIM-3, LAG-3, and CXCL13;
[11] the method of any one of [7]-[10], wherein the sample is a bone marrow or PBMC sample;
[12] the method of any one of [7]-[11], wherein the sample is a serum sample;
[13] the method of any one of [7]-[12], wherein the change in the level of a set of biomarkers is an increase of an expression level of each of the biomarkers in the set;
[14] a method of preventing, treating or ameliorating a disease or disorder caused by a pathogenic bacterium or a pathogenic fungus in a subject in need thereof, comprising administering to the subject a therapeutically effective amount of an inhibitor of an inhibitory immune checkpoint molecule;
[15] a method of preventing, treating or ameliorating a disease or disorder caused by a pathogenic bacterium or a pathogenic fungus in a subject in need thereof, comprising administering a therapeutically effective amount of an inhibitor of an inhibitory immune checkpoint molecule], wherein the subject is determined to have a disease or disorder caused by the pathogenic bacterium or pathogenic fungus according to the method of any one of [7]-[10];
[16] the method of any one of [7]-[15], wherein the disease or disorder comprises an infection;
[17] the method of [16], wherein the infection is bacteremia, a bone infection, osteomyelitis, or sepsis;
[18] the method of [16], wherein the infection is a prosthetic joint infection, a fracture related infection, a diabetic foot infection, a hematogenous osteomyelitis, or a spine infection;
[19] the method of any one of [14]-[18], wherein the administered inhibitor inhibits an inhibitory immune checkpoint molecule selected from the group consisting of LAG-3, TIM-3, CTLA-4, PD-1, and PD-L1;
[20] the method of any one of [14]-[19], wherein the administered inhibitor comprises an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof;
[21] the method of any one of [14]-[20], wherein the administered inhibitor comprises an anti-LAG-3 antibody, an anti-TIM-3 antibody, an anti-CTLA-4 antibody, an anti-PD-1 antibody, an anti-PD-L1 antibody, an antigen-binding fragment thereof, or a combination thereof;
[22] the method of any one of [14]-[21], wherein the administered inhibitor comprises (i) an anti-LAG-3 antibody or an antigen-binding fragment thereof, (ii) a combination of an anti-PD-1 antibody or an antigen-binding fragment thereof and an anti-LAG-3 antibody or an antigen-binding fragment thereof, or (iii) a bispecific antibody that binds PD-1 and LAG-3;
[23] the method of [22], wherein the anti-PD-1 antibody is nivolumab, the anti-LAG-3 antibody is relatlimab, or the bispecific antibody is an anti-PD-1 and anti-LAG-3 bispecific antibody (e.g., a bispecific antibody comprising an antigen binding fragment of nivolumab and an antigen binding fragment of relatlimab);
[24] the method of any one of [1]-[6] and [14]-[23], which further comprises administering to the subject an additional therapeutic agent;
[25] the method of [24], wherein the additional therapeutic agent comprises a second inhibitor of a second inhibitory immune checkpoint molecule, an antibiotic, an antipathogen antibody specific for the pathogenic bacterium or pathogenic fungus, or a combination thereof;
[26] the method of [25], wherein the additional administered therapeutic agent is an antipathogen antibody that specifically binds to Staphylococcus aureus;
[27] the method of [25], wherein the additional administered therapeutic agent is a second inhibitory immune checkpoint molecule selected from the group consisting of LAG-3, TIM-3, CTLA-4, PD-1, and PD-L1;
[28] the method of [27], wherein the additional administered therapeutic agent comprises an anti-LAG-3 antibody, an anti-TIM-3 antibody, an anti-CTLA-4 antibody, an anti-PD-1 antibody, an anti-PD-L1 antibody, or a combination thereof;
[29] the method of [24], wherein the additional administered therapeutic agent is an antibiotic having an anti-bacterial activity against Staphylococcus aureus;
[30] the method of any one of [24]-[29], wherein the additional therapeutic agent is administered concurrently with the inhibitor;
[31] the method of any one of [24]-[29], wherein the additional therapeutic agent is administered before or after the inhibitor;
[32] the method of or [30], wherein the inhibitor and the additional therapeutic agent are contained in the same composition;
[33] the method of any one of [24]-[32], wherein the inhibitor and/or the additional therapeutic agent is administered to the subject intratumorally, intravenously, subcutaneously, intraosseously, orally, transdermally, in sustained release, in controlled release, in delayed release, as a suppository, or sublingually;
[34] the method of any one of [7]-[33], wherein the subject has received or is about to receive a surgery or an implant;
[35] the method of any one of [7]-[34], wherein the subject has received surgery;
[36] the method of any one of [7]-[34], wherein the subject is about to receive surgery;
[37] the method of any one of [34]-[36], wherein the surgery is selected from the group consisting of orthopedic surgery, cardiothoracic surgery, plastic surgery, neurosurgery, oral surgery, total joint replacement, open reduction internal fixation (ORIF), debridement for open fracture, spine surgery, median sternotomy, revision total joint, revision ORIF, drainage of soft tissue abscess, and organ transplantation surgery;
[38] the method of any one of [34], or [37], wherein the subject has received an implant;
[39] the method of any one of or [36], wherein the subject is about to receive an implant;
[40] the method of any one of [34]-[39], wherein the subject has received or is about to receive an orthopedic implant;
[41] the method of any one of [1]-[40], wherein the bacterium is selected from the group consisting of Staphylococcus aureus, S. epidermidis, S. lugdunensis, Cutibacterium acnes, Group B Streptococcus, and Enterobacteria;
[42] the method of [41], wherein the Staphylococcus aureus is methicillin-resistant Staphylococcus aureus (MRSA) or methicillin-susceptible Staphylococcus aureus (MSSA);
[43] the method of any one of [1]-[42], wherein the subject is a mammal;
[44] the method of any one of [1]-[43], wherein the mammal is a human;
[45] use of one or more inhibitors of inhibitory immune checkpoint molecules in manufacture of a medicament for the method of any one of [1]-[6] and [14]-[44];
[46] one or more inhibitors of inhibitory immune checkpoint molecules for use in the method of any one of [1]-[6] and [14]-[45];
[47] a method for reducing the number of cells of a pathogenic bacterium or a pathogenic fungus in a subject in need thereof, comprising administering an effective amount of an inhibitor of an inhibitory immune checkpoint molecule to a subject determined to have
   (i) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 pg/ml (e.g., as determined, in a method described herein or a method known in the art);
[48] a method of preventing, treating or ameliorating a disease or disorder caused by a pathogenic bacterium or a pathogenic fungus in a subject in need thereof, comprising administering to the subject a therapeutically effective amount of an inhibitor of an inhibitory immune checkpoint molecule to a subject determined to have
   (i) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml (e.g., as determined, in a method described herein or a method known in the art);
[49] the method of [48], wherein the disease or disorder comprises an infection and the infection is a prosthetic joint infection, a fracture related infection, a diabetic foot infection, a hematogenous osteomyelitis, or a spine infection;
[50] a method of treating or preventing a bone infection or bone loss in a subject in need thereof, comprising administering an effective amount of an inhibitor of an inhibitory immune checkpoint molecule;
[51] the method of [50], wherein the method treats or prevents bone infection by a pathogenic bacterium or a pathogenic fungus;
[52] the method of [51], wherein the bone infection is osteomyelitis;
[53] the method of or [52], wherein the method prevents bone loss associated with an infection by a pathogenic bacterium or a pathogenic fungus;
[54] A method of treating or preventing sepsis associated with a pathogenic bacterium or a pathogenic fungus in a subject in need thereof, comprising administering an effective amount of an inhibitor of an inhibitory immune checkpoint molecule;
[55] the method of [54], wherein the method treats or prevents S. aureus associated sepsis;
[56] A method of treating or preventing bacteremia, biofilm formation, or the development of an antimicrobial resistant infection in a subject in need thereof, comprising administering an effective amount of an inhibitor of an inhibitory immune checkpoint molecule;
[57] the method of any one of [1]-[6], [49]-[56], wherein prior to the administration of the inhibitor, the subject is determined to have:
   (i) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml (e.g., as determined, in a method described herein or a method known in the art);
[58] the method of any one of [49]-[56], wherein prior to a surgery or receiving an implant (e.g., within 1 year, 9 months, 6 months, 3 months 1 month or 2 weeks), the subject is determined to have:
   (i) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml (e.g., as determined, in a method described herein or a method known in the art);
[59] the method of any one of [1]-[6] and [42]-[58], wherein the subject is administered the inhibitor prior to surgery or receiving an implant;
[60] the method of any one of [1]-[6] and [42]-[57], wherein the subject is administered the inhibitor after a surgery or receiving an implant;
[61] the method of any one of [1]-[6], and [42]-[59], wherein the subject has received surgery within 1 week, 2 weeks, 3 weeks, 1 month 3 months, 6 months, 12 months, or 18 months after the administration of the inhibitor of the inhibitory immune checkpoint molecule;
[62] the method of any one of [1]-[6], [42]-[57], and [60], wherein the subject has received surgery within 1 week, 2 weeks, 3 weeks, 1 month 3 months, 6 months, 12 months, or 18 months prior to administration of the inhibitor of the inhibitory immune checkpoint molecule;
[63] the method of any one of [57]-[62], wherein the surgery is selected from the group consisting of orthopedic surgery, cardiothoracic surgery, plastic surgery, neurosurgery, oral surgery, total joint replacement, open reduction internal fixation (ORIF), debridement for open fracture, spine surgery, median sternotomy, revision total joint, revision ORIF, drainage of soft tissue abscess, or organ transplantation surgery;
[64] the method of any one of [1]-[6], [42]-[59], [63 [61], and [63], wherein the subject has received an implant within 1 week, 2 weeks, 3 weeks, 1 month 3 months, 6 months, 12 months, or 18 months after the administration of the inhibitor of the inhibitory immune checkpoint molecule;
[65] the method of any one of [1]-[6], [42]-[57], [60], [62], and [63], wherein the subject has received an implant within 1 week, 2 weeks, 3 weeks, 1 month 3 months, 6 months, 12 months, or 18 months prior to administration of the inhibitor of the inhibitory immune checkpoint molecule;
[66] the method of any one of [59]-[65], wherein implant is an orthopedic implant;
[67] the method of any one of [14]-[44], [47]-[66], wherein the administered inhibitor comprises an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof;
[68] the method of any one of [1]-[6], [14]-[44], and [47]-[68], wherein the serum titer of soluble TIM-3 in the subject is determined to be elevated compared to a previously measured TIM-3 serum titer for the subject;
[69] the method of any one of [1]-[6], [14]-[44], and [47]-[68], wherein the serum titer of soluble TIM-3 in the subject is determined to be at least 10% higher in the subject than in healthy control subjects;
[70] the method of [1]-[6], [14]-[44], and [47]-[69], wherein the serum titer of soluble TIM-3 in the subject is determined to be at least 20%, 30%, 40%, 50%, 60%, 70%, 80%, 90% or 100% (2 fold) higher in the subject than that in healthy control subjects;
[71] the method of any one of [1]-[6], [14]-[44], and [47]-[70], wherein the serum titer of soluble TIM-3 in the subject is determined to be at least 2100, 2200, 2300, 2400, or 2500 pg/ml (as determined, e.g., in a method described in the Examples);
[72] the method of any one of [1]-[6], [14]-[44], and [47]-[71], wherein the serum titer of soluble TIM-3 in the subject is determined to be at least 3000 μg/ml (as determined, e.g., in a method described herein);
[73] the method of any one of [1]-[6], [14]-[44], and [47]-[72], wherein the soluble TIM-3 is differentially spliced soluble TIM-3 and/or shed TIM-3;
[74] the method of any one of [47]-[49 and 57]-[70], wherein the serum titer of soluble TIM-3 in healthy control subjects is the mean or average titer of soluble TIM-3 in at least 10, 50 or 100 subjects;
[75] a method for reducing the number of cells of a pathogenic bacterium or a pathogenic fungus in a subject in need thereof, comprising administering comprising administering an effective amount of an inhibitor of an inhibitory immune checkpoint molecule to a subject determined to have
   (i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 at least 80000, 85000, 90000, 95000, or 100000 μg/ml (e.g., as determined, in a method described herein or a method known in the art);
[76] a method of preventing, treating or ameliorating a disease or disorder caused by a pathogenic bacterium or a pathogenic fungus in a subject in need thereof, comprising administering to the subject a therapeutically effective amount of an inhibitor of an inhibitory immune checkpoint molecule to a subject determined to have
   (i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 at least 80000, 85000, 90000, 95000, or 100000 μg/ml (e.g., as determined, in a method described herein or a method known in the art);
[77] the method of [76], wherein the disease or disorder comprises an infection and the infection is a prosthetic joint infection, a fracture related infection, a diabetic foot infection, a hematogenous osteomyelitis, or a spine infection;
[78] a method of treating or preventing a bone infection or bone loss in a subject in need thereof, comprising administering an effective amount of an inhibitor of an inhibitory immune checkpoint molecule;
[79] the method of [78], wherein the method treats or prevents bone infection by a pathogenic bacterium or a pathogenic fungus;
[80] the method of or [79], wherein the bone infection is osteomyelitis;
[81] the method of any one of [78]-[80], wherein the method prevents bone loss associated with an infection by a pathogenic bacterium or a pathogenic fungus;
[82] a method of treating or preventing sepsis associated with a pathogenic bacterium or a pathogenic fungus in a subject in need thereof, comprising administering an effective amount of an inhibitor of an inhibitory immune checkpoint molecule;
[83] the method of [82], wherein the method treats or prevents S. aureus associated sepsis;
[84] a method of treating or preventing bacteremia, biofilm formation, or the development of an antimicrobial resistant infection in a subject in need thereof, comprising administering an effective amount of an inhibitor of an inhibitory immune checkpoint molecule;
[85] the method of any one of [77]-[84], wherein prior to the administration the subject is determined to have:
   (i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml (e.g., as determined, in a method described herein or known in the art);
[86] the method of any one of [75]-[85], wherein the serum titer of soluble LAG-3 in the subject is determined to be elevated compared to a previously measured LAG-3 serum titer for the subject;
[87] the method of any one of [75]-[86], wherein the serum titer of soluble LAG-3 in the subject is determined to be at least 10% higher in the subject than in healthy control subjects;
[88] the method of any one of [1]-[6], [14]-[44], or [47]-[87], wherein the serum titer of soluble LAG-3 in the subject is determined to be at least 20%, 30%, 40%, 50%, 60%, 70%, 80%, 90% or 100% (2 fold) higher in the subject than that in healthy control subjects;
[89] the method of any one of [1]-[6], [14]-[44], or [47]-[88], wherein the serum titer of soluble LAG-3 in the subject is determined to be at least at least 80000, 85000, 90000, 95000, or 100000 μg/ml (as determined, e.g., in a method described in the Examples);
[90] the method of any one of [1]-[6], [14]-[44], or [47]-[89], wherein the serum titer of soluble LAG-3 in the subject is determined to be at least 90000 μg/ml (as determined, e.g., in a method described herein or accepted standard);
[91] the method according to any one of [75]-[90], wherein the soluble LAG-3 is differentially spliced soluble LAG-3 and/or shed LAG-3;
[92] the method of any one of [75], [76], [85], [87], or [88], wherein the serum titer of soluble LAG-3 in healthy control subjects is the mean or average titer of soluble LAG-3 in at least 10, 50 or 100 subjects;
[93] a method for reducing the number of cells of a pathogenic bacterium or a pathogenic fungus in a subject in need thereof, comprising administering an effective amount of an inhibitor of an inhibitory immune checkpoint molecule to a subject determined to have
   (i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml (e.g., as determined, in a method described herein or a method known in the art);
[94] a method of preventing, treating or ameliorating a disease or disorder caused by a pathogenic bacterium or a pathogenic fungus in a subject in need thereof, comprising administering to the subject a therapeutically effective amount of an inhibitor of an inhibitory immune checkpoint molecule to a subject determined to have
   (i) an elevated serum titer of CXCL13 compared to a previously measured CXCL 13 serum titer for the subject,
   (ii) a serum titer of CXCL 13 higher than that in healthy control subjects, or
   (iii) a serum titer of CXCL13 is at least 50, 55, 60, 65, 70, or 75 μg/ml (e.g., as determined, in a method described herein or a method known in the art);
[95] the method of any one of or [94], wherein prior to administration of the inhibitor the subject is further determined to have
   (i) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml (e.g., as determined, in a method described herein or a method known in the art);
[96] the method of any one of [93]-[95], wherein prior to administration of the inhibitor the subject is further determined to have
   (i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 at least at least 80000, 85000, 90000, 95000, or 100000 μg/ml (e.g., as determined, in a method described herein or a method known in the art);
[97] the method of any one of [94]-[96], wherein the disease or disorder comprises;
   (a) an infection such as a bone infection (e.g., a bone infection by a pathogenic bacterium or a pathogenic fungus), osteomyelitis, a prosthetic joint infection, a fracture related infection, a diabetic foot infection, a hematogenous osteomyelitis, or a spine infection;
   (b) sepsis associated with a pathogenic bacterium or a pathogenic fungus such as, S. aureus associated sepsis,
   (c) bacteremia,
   (d) biofilm formation, or
   (e) the development of an antimicrobial resistant infection;
[98] the method of any one of [93]-[97], wherein prior to the administration of the inhibitor the subject is determined to have:
(a)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL 13 is at least is at least 50, 55, 60, 65, 70, or 75 μg/ml (e.g., as determined, in a method described herein or a method known in the art);
(b)(i) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml (e.g., as determined, in a method described herein or a method known in the art); and/or
(c)(i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml (e.g., as determined, in a method described herein or known in the art); or any combination of (a)-(c);
[99] the method of any one of [98]-[105], wherein prior to a surgery or receiving an implant (e.g., within 1 year, 9 months, 6 months, 3 months 1 month or 2 weeks), the subject is determined to have:
(a)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml (e.g., as determined, in a method described herein or a method known in the art);
(b)(i) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml (e.g., as determined, in a method described herein or a method known in the art); and/or
(c)(i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml (e.g., as determined, in a method described herein or known in the art); or any combination of (a)-(c);
[100] the method of [99], wherein the subject has received surgery within 1 week, 2 weeks, 3 weeks, 1 month 3 months, 6 months, 12 months, or 18 months prior to the administration of the inhibitor of the inhibitory immune checkpoint molecule;
[101] the method of or [100], wherein the subject has received an implant within 1 week, 2 weeks, 3 weeks, 1 month 3 months, 6 months, 12 months, or 18 months prior to the administration of the inhibitor of the inhibitory immune checkpoint molecule;
[102] the method of any one of [98]-[105], wherein after surgery or receiving an implant, the subject is determined to have:
(a)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml (e.g., as determined, in a method described herein or a method known in the art);
(b)(i) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml (e.g., as determined, in a method described herein or a method known in the art); and/or
(c)(i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 pg/ml (e.g., as determined, in a method described herein or known in the art); or any combination of (a)-(c);
[103] the method of [102], wherein the subject has received surgery within 1 week, 2 weeks, 3 weeks, 1 month 3 months, 6 months, 12 months, or 18 months after the administration of the inhibitor of the inhibitory immune checkpoint molecule;
[104] the method of or [103], wherein the subject has received an implant within 1 week, 2 weeks, 3 weeks, 1 month 3 months, 6 months, 12 months, or 18 months after the administration of the inhibitor of the inhibitory immune checkpoint molecule;
[105] the method of any one of [99]-[104], wherein the surgery is selected from the group consisting of orthopedic surgery, cardiothoracic surgery, plastic surgery, neurosurgery, oral surgery, total joint replacement, open reduction internal fixation (ORIF), debridement for open fracture, spine surgery, median sternotomy, revision total joint, revision ORIF, drainage of soft tissue abscess, or organ transplantation surgery;
[106] the method of any one of [99]-[105], wherein implant is an orthopedic implant;
[107] the method of any one of [68]-[106], wherein the administered inhibitor comprises an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof;
[108] the method of any one of [1]-[6], [14]-[44], and [46]-[107], wherein the serum titer of CXCL13 in the subject is determined to be elevated compared to a previously measured CXCL13 serum titer for the subject;
[109] the method of any one of [1]-[6], [14]-[44], and [46]-[108], wherein the serum titer of CXCL 13 in the subject is determined to be at least 10% higher in the subject than in healthy control subjects;
[110] the method of any one of [1]-[6], [14]-[44], and [46]-[109], wherein the serum titer of CXCL13 in the subject is determined to be at least 20%, 30%, 40%, 50%, 60%, 70%, 80%, 90% or 100% (2 fold) higher in the subject than that in healthy control subjects;
[111] the method of any one of [11]-[6], [14]-[44], and [46]-[110], wherein the serum titer of CXCL13 in the subject is determined to be at least 2100, 2200, 2300, 2400, or 2500 pg/ml (as determined, e.g., in a method described in the Examples);
[112] the method of any one of [1]-[6], [14]-[44], and [46]-[111], wherein the serum titer of CXCL13 in the subject is determined to be at least 3000 μg/ml (as determined, e.g., in a method described herein;
[113] the method of any one of [98]-[11], wherein the serum titer of CXCL13 in healthy control subjects is the mean or average titer of CXCL13 in at least 10, 50 or 100 subjects;
[114] the method of any one of [46]-[113], wherein the administered inhibitor is a LAG-3 inhibitor, a TIM-3 inhibitor, a TIGIT inhibitor, a PD-1 inhibitor, or a PD-L1 inhibitor;
[115] the method of any one of [46]-[114], wherein the administered inhibitor is an antibody or an antigen-binding fragment thereof;
[116] the method of any one of [1]-[6], [14]-[44], and [46]-[115], wherein the administered inhibitor is a LAG-3 inhibitor;
[117] the method of [116], wherein the administered inhibitor is a LAG-3 antibody or an antigen-binding fragment thereof;
[118] the method of [117], wherein the administered LAG-3 antibody or antigen-binding fragment thereof is an antibody or antigen-binding fragment of an antibody selected from the group consisting of BMS 986016, MK-4280 (28G-10), REGN3767, GSK2831781, IMP731 (H5L7BW), BAP050, IMP-701 (LAG-5250), TSR-033, LAG525, BI754111, and FS-118;
[119] the method of [117], wherein the administered LAG-3 antibody or antigen-binding fragment thereof is an antibody or antigen-binding fragment of an antibody selected from the group consisting of relatlimab, BMS-986016, and GSK2831781.
[120] the method of any one of [1]-[6], [14]-[44], or [46]-[115], wherein the administered inhibitor is a TIM-3 inhibitor;
[121] the method of [120], wherein the administered inhibitor is a TIM-3 antibody or an antigen-binding fragment thereof;
[122] the method of [121], wherein the administered TIM-inhibitor is an antibody or antigen-binding fragment of an antibody selected from the group consisting of TSR-022, LY3321367, EPZ005687, and DZNep;
[123] the method of [121], wherein the administered TIM-3 inhibitor is an antibody or antigen-binding fragment of an antibody selected from the group consisting of TSR-022 and LY3321367;
[124] the method of any one of [1]-[6], [14]-[44], and [46]-[115], wherein the administered inhibitor is a TIGIT inhibitor;
[125] the method of [124], wherein the administered inhibitor is a TIGIT antibody or an antigen-binding fragment thereof;
[126] the method of [125], wherein the administered TIGIT inhibitor is an antibody or antigen-binding fragment of an antibody selected from the group consisting of BMS-986207, AB154, COM902 (CGEN-15137), or OMP-313M32;
[127] the method of [125], wherein the administered TIGIT inhibitor is an antibody or antigen-binding fragment of an antibody selected from the group consisting of BMS-986207 and AB154;
[128] the method of any one of [1]-[6], [14]-[44], and [46]]-[115], wherein the administered inhibitor is a PD-1 inhibitor;
[129] the method of [128], wherein the administered inhibitor is a PD-1 antibody or an antigen-binding fragment thereof;
[130] the method of [129], wherein the administered PD-1 inhibitor is an antibody or antigen-binding fragment of an antibody selected from the group consisting of nivolumab, pembrolizumab, pidilizumab, nivolumab, PDR001, MEDI0680 (AMP-514), TSR-042, REGN2810, JS00I, AMP-224 (GSK-2661380), PF-06801591, BGB-A317, BI754091, and SHR-1210;
[131] the method of [129], wherein the administered PD-1 inhibitor is an antibody or antigen-binding fragment of an antibody selected from the group consisting of nivolumab, pembrolizumab, pidilizumab, nivolumab, BGB-A317, AMP-224, PDR001, and MEDI0680 (AMP-514);
[132] the method of any one of [1]-[6], [14]-[44], and [46]-[115], wherein the administered inhibitor is a PD-L1 inhibitor;
[133] the method of [132], wherein the administered inhibitor is a PD-L1 antibody or an antigen-binding fragment thereof;
[134] the method of [133], wherein the administered PD-L1 inhibitor is an antibody or antigen-binding fragment of an antibody selected from the group consisting of atezolizumab, durvalumab, BMS-936559, avelumab, LY3300054, CX-072 (Proclaim-CX-072), FAZ053, KN035, MPDL3280A, MEDI4736, MSB0010718C and MDX-1105;
[135] the method of [133], wherein the administered PD-L1 inhibitor is an antibody or antigen-binding fragment of an antibody selected from the group consisting of atezolizumab, durvalumab, BMS-936559, MPDL3280A, MEDI4736, MSB0010718C and avelumab;
[136] the method of [132], wherein the administered inhibitor is a small molecule;
[137] the method of [136], wherein the administered inhibitor is a small molecule selected from the group consisting of BMS-8, BMS-37, BMS-202, BMS-230, BMS-242, BMS-1001, BMS-1166, SB415286, JQI, and I-BET151;
[138] the method of any one of [1]-[6], [14]-[44], and [46]-[137], wherein the administered inhibitor is a bispecific antibody or a soluble immune checkpoint molecule binding ligand (e.g. a CTLA4-Fc fusion protein) that binds at least one immune checkpoint inhibitory molecule;
[139] the method of [138], wherein the administered bispecific antibody binds 2 different inhibitory immune checkpoint molecules;
[140] the method of [139], wherein the bispecific antibody binds PD-1 and LAG-3, or PD-1 and TIM-3;
[141] the method of [139], wherein the bispecific antibody comprises antigen binding fragments of nivolumab and relatlimab;
[142] the method of any one of [46]-[141], which further comprises administering to the subject an additional therapeutic agent;
[143] the method of [142], wherein the additional therapeutic agent is a second inhibitor of a second inhibitory immune checkpoint molecule, an antibiotic, an antipathogen antibody specific for the pathogenic bacterium or pathogenic fungus, or a combination thereof;
[144] the method of [143], wherein the additional administered therapeutic agent is an antipathogen antibody specifically binds to Staphylococcus aureus;
[145] the method of [143], wherein the additional administered therapeutic agent is a second administered inhibitory immune checkpoint molecule selected from the group consisting of LAG-3, TIM-3, CTLA-4, PD-1, and PD-L1;
[146] the method of or [145], wherein the second administered inhibitor of an inhibitory immune checkpoint molecule is an anti-LAG-3 antibody, an anti-TIM-3 antibody, an anti-CTLA-4 antibody, an anti-PD-1 antibody, an anti-PD-L1 antibody, or a combination thereof;
[147] the method of [143], wherein the additional administered therapeutic agent is an antibiotic having an anti-bacterial activity against Staphylococcus aureus;
[148] the method of any one of [143]-[147], wherein the additional therapeutic agent is administered concurrently with the inhibitor;
[149] the method of any one of [143]-[147], wherein the additional therapeutic agent is administered before or after the inhibitor;
[150] the method of any one of [143]-[148], wherein the inhibitor and the additional therapeutic agent are contained in the same composition;
[151] the method of any one of [143]-[150], wherein the inhibitor and/or the additional therapeutic agent is administered to the subject intratumorally, intravenously, subcutaneously, intraosseously, orally, transdermally, in sustained release, in controlled release, in delayed release, as a suppository, or sublingually;
[152] the method of any one of [142]-[151], wherein the subject has received or is about to receive a surgery or an implant;
[153] the method of any one of [46]-[152], wherein the bacterium is selected from the group consisting of Staphylococcus aureus, S. epidermidis, S. lugdunensis, Cutibacterium acnes, Group B Streptococcus, and Enterobacteria;
[154] the method of [153], wherein the Staphylococcus aureus is methicillin-resistant Staphylococcus aureus (MRSA) or methicillin-susceptible Staphylococcus aureus (MSSA);
[155] the method of any one of [47]-[154], wherein the subject is a mammal;
[156] the method of any one of [47]-[155], wherein the mammal is a human;
[157] use of one or more inhibitors of inhibitory immune checkpoint molecules in manufacture of a medicament for the method of any one of [47]-[156]; and/or
[158] one or more inhibitors of inhibitory immune checkpoint molecules for use in the method of any one of [47]-[156].

In some embodiments, the bacterium is selected from the group consisting of Staphylococcus aureus, S. epidermidis, S. lugdunensis, Cutibacterium acnes, Group B Streptococcus, and Enterobacteria. In some embodiments, the bacterium is selected from the group consisting of S. saprophyticus, S. haemolyticus, S. caprae, and S. simiae. In some embodiments, the S. aureus is methicillin-resistant S. aureus (MRSA) or methicillin-susceptible S. aureus (MSSA). In some embodiments, the S. aureus is resistant to one or more b-lactam antimicrobial agents. In some embodiments, the S. aureus is vancomycin resistant.

In some embodiments, the subject is a mammal. In some embodiments, the mammal is a human.

Also within the scope of this disclosure are (a) use of one or more inhibitors of immune checkpoint molecules in manufacture of a medicament for the method described herein, and (b) one or more inhibitors of immune checkpoint molecules for use in the method described herein.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if combinations of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A, 1B, 1C, 1D, 1E, IF, 1G, 1H, 1I, 1J, and 1K show that humanized NSG-SGM3 BLT mice have exacerbated susceptibility to S. aureus compared to murinized NSG-SGM3 and C57BL/6 WT mice. FIG. 1A shows that humanized NSG-SGM3 BLT mice were generated by engrafting with CD34+ human hematopoietic cells, autologous human fetal liver, and thymus from three different human donors. Murinized NSG-SGM3 BLT mice were generated with CD34+ murine hematopoietic cells derived from three different C57BL/6 WT mice. FIG. 1B shows schematic illustration of the experimental design of in vivo experiments. 20-week-old humanized NSG-SGM3 BLT mice, murinized NSG-SGM3 and C57BL6 (WT) mice were subjected to transtibial implant-associated osteomyelitis using bioluminescent MRSA (USA300 LAC::lux). FIGS. 1C and 1D show longitudinal assessments of in vivo S. aureus growth via bioluminescent imaging revealing increased in vivo S. aureus growth in humanized NSG-SGM3 BLT mice. FIG. 1E shows MRSA dissemination from the site of infection to distal internal organs. FIGS. F, 1G, 1H, and 1I show that on day 14 post-operation, huNSG-SGM3 BLT mice and control animals were euthanized, and ex vivo CFU quantification was performed on the implants, tibia, soft tissues surrounding the tibia, and internal organs (heart, liver, kidneys, and spleen). Interestingly, CFU quantitation revealed that huNSG-SGM3 BLT mice exhibited exacerbated susceptibility and increased sepsis due to S. aureus osteomyelitis compared to control murinized NSG-SGM3 and C57BL6 (WT) animals (n=25 (3 human donors), ANOVA, *p<0.05, ** p<0.01, *** p<0.001, *** p<0.0001). Post euthanasia, MRSA-infected and sterile implant-inserted tibiae from a subset of huNSG-SGM3 BLT, NSG-SGM3, and C57BL/6 animals were demineralized, sectioned, and processed for histopathological analyses. FIGS. 1J and 1K show Brown & Brenn (B&B) staining for identifying bacteria revealed numerous SACs formation and increased SAC area over the tibial area in humanized BLT mice, compared to control animals (n=4-5, ANOVA, *p<0.05).

FIGS. 2A, 2B, 2C, 2D, 2E, and 2F show that single-cell RNAseq reveals remarkable human T cell heterogeneity in numbers and gene expression due to S. aureus osteomyelitis in humanized BLT mice. (FIG. 2A) Schematic illustration showing the experimental overview of single-cell RNAseq of humanized NSG-SGM3 BLT mice engrafted with three different human donor tissues. Bone marrow (BM) cells were isolated from tibiae of humanized NSG-SGM3 BLT mice that underwent surgery with or without bioluminescent MRSA-contaminated transtibial implant on day 14 post-operation. Isolated BM cells were FACS sorted into human CD45+CD19+B cells and CD45+CD3+ T cells. As depicted here, equal proportions of the B and T cells were subjected to sc-RNAseq and sc-TCR/BCR repertoire analyses. (FIG. 2B) UMAP plot of single-cell gene expression of T and B cells in all ~30,000 BM cells from humanized NSG-SGM3 BLT mice tibiae. (FIG. 2C) Feature plots of pan T cell marker, CD3E and B cell marker, CD19 in all integrated BM cells. (FIG. 2D-2E) UMAP and DEG clustering analyses of hCD45+ CD3+ T cells identified 24 T cell clusters (FIG. 2F) Bar plot displaying the proportion of cell counts in each cluster between sterile sham surgery and infected implant groups. Interestingly, the number of Th1/Th17 cells (Cluster 8,20) was prominently increased in the infected animals compared to sham surgery (sterile) animals.

FIGS. 3A, 3B, 3C, 3D, and 3E show that immune checkpoint proteins are elevated in the CD4+Th1/Th17 cells in S. aureus-infected humanized BLT mice tibia. (FIG. 3A) Th1/Th17 cells (clusters 8 and 20) from FIG. 3 subjected to UMAP and differential gene expression analyses (DEG) sub-clustering analyses revealed 7 clusters. (FIG. 3B) Bar plot analyses demonstrated that these cells were of the Th1/Th17 phenotype. Several Th1/Th17 clusters showed significantly increased expression of immune checkpoint molecules LAG3, TIM-3 (HAVCR2), and, to a lesser extent, CTLA-4 and other immunosuppressive genes like TIGIT. (FIG. 3C) DEG analyses of transcriptional factors (TCF7, TOX1-2, EOMES, NR4A1), cytokines & chemokines (IL-1, IL-17, CXCL13, CXCR5) associated with functional T cell exhaustion, chronic antigenic stimulation (CD40L) and proliferation (MKi67). Note that the lower expression of TCF7, MKi67, IL-1, and IL-17 genes and higher expression of CXCL13 and TOX 2 indicate transcriptional reprogramming of these cells to a terminally functionally exhausted state (*p<0.05). (FIG. 3D-E) Immunofluorescent analyses of tibia sections from sham surgery control and humanized BLT mice show a large accumulation of LAG3+, TIM-3+, and/or PD-1+ T cells near the infection site in the MRSA-infected BLT mice. In contrast, human T cell accumulation was scant in uninfected BLT mice, and there was evidence of minimal exhaustion observed near the infection site. (FIG. 3E) A multichromatic spectral flow cytometry assay was developed, optimized, and performed on uninfected and MRSA-infected BLT mice's tibial bone marrow cells (BMs). Live human CD45+/CD3+/T cells and their subpopulations (CD4+, CD8+, Tregs) were analyzed for immune checkpoint expression (LAG3, TIM-3, and PD-1) and proliferation (Ki67). Note the frequency of human CD3+CD4+ T cells expressing TIM-3, LAG3 & PD-1) in the MRSA-infected BLT mice bone marrow cells were significantly higher compared to controls (n=4-8 mice, *p<0.05, t-test).

FIGS. 4A and 4B show that splenic and bone marrow CD4+ T cells expressing TIM-3 and LAG3 checkpoint proteins exhibit diminished proliferative capacity due to S. aureus infection. Multichromatic spectral flow cytometry on uninfected and MRSA-infected BLT mice (FIG. 4A) splenic cells and (FIG. 4B) tibial bone marrow cells was performed using protocols described herein. Subsequently, CD4+ T cell subpopulations expressing checkpoint molecules TIM-3, LAG3, and PD-1 were probed for their proliferative capacity using the cell surface marker Ki67. Note that CD4+TIM-3+ and CD4+LAG3+ cells have a significantly lower frequency of proliferating Ki67+ cells in the spleen and trending lower amounts of proliferating Ki67+ cells in the bone marrow of infected BLT mice, suggesting functional exhaustion and dysfunction (n=4-9 mice, *p<0.05, ANOVA).

FIGS. 5A, 5B, and 5C show that serum immune checkpoint proteins are highly prognostic of adverse outcomes in patients with S. aureus osteomyelitis. FIG. 5A shows serum samples that were collected from individuals undergoing total hip/knee arthroplasty (n=15), orthopedic patients with culture-confirmed S. aureus osteomyelitis with one-year (n=37, 12-Adverse Outcome (AD), 11-Infection Controlled (IC), 14-inconclusive). Immune checkpoint proteins LAG-3, TIM-3, CTLA-4, PD-1 and cytokines (IFN-γ, IL-2, TNFα, IL-17A, IL-17F) were assessed by multiplex Luminex assay. Data presented as the mean +/−SEM in each experimental group. The individual protein levels were utilized to perform receiver operating characteristic (ROC) curve analysis either singly or in combination to generate the area under the curve (AUC) for (FIG. 5B) differentiating acute vs. chronic S. aureus infections and (FIG. 5C) prognostic prediction of outcome. The no-correlation was observed between levels of immune checkpoint proteins and clinical time-based, anecdotal classification of acute vs. chronic classification. On the other hand, immune checkpoint proteins, especially TIM-3, were highly predictive of adverse in these patients (*p<0.05, ** p<0.01, **** p<0.00001).

FIGS. 6A, 6B, 6C, 6D, and 6E shown preliminary feasibility data demonstrating an efficacy trend of immune checkpoint blockade drug OPDUALAG™ (anti-PD-1 (nivolumab) +anti-LAG3 (relatlimab) mAb cocktail) in reducing S. aureus burden in humanized BLT mice. (FIG. 6A) Schematic illustration of the experimental design. 20-week-old humanized NSG-SGM3 BLT mice were subjected to transtibial implant-associated osteomyelitis using bioluminescent MRSA (USA300 LAC::lux) and treated with OPDUALAG™ or a saline placebo control. The concentration of anti-PD1 and anti-LAG3 mAbs used in the study is also outlined. (FIG. 6B) Longitudinal BLI revealed significantly decreased in vivo S. aureus growth in humanized BLT mice treated with OPDUALAG™ (n=3-4, ANOVA, *p<0.05). At day 14 post-surgery, terminal ex vivo CFU on the tibia and internal organs (heart, liver, kidneys, and spleen) revealed that OPDUALAG™-treated animals exhibited trending lower disease severity. (FIG. 6C) Interestingly, histopathology revealed remarkably reduced SACs formation in the OPDUALAG™-treated animals compared to controls. (FIG. 6D) μCT analysis revealed reduced bone reaction in the OPDUALAG™-treated animal (the rough, pitted area around the implant hole (red arrows)), and the blue color indicates the hole formed at the site of infection due to the infected implant. (FIG. 6E) Immunofluorescent micrographs depicting pronounced reduction in LAG3+ and PD-1+ T cells in the OPDUALAG™-treatment animal. Additionally, increased proliferating PCNA+ T cells were observed in the OPDUALAG™-treatment animal, potentially suggesting functional restoration of exhausted T cells.

FIGS. 7A, 7B, 7C, and 7D show preliminary feasibility data demonstrating an efficacy trend of immune checkpoint blockade drug Sabatolimab (anti-TIM-3) in reducing S. aureus burden due to implant-associated osteomyelitis. (FIG. 7A) Schematic illustration of the experimental design. 8-week-old C57BL/6 mice were subjected to transtibial implant-associated osteomyelitis using bioluminescent MRSA (USA300 LAC::lux) and treated with sabatolimab or a saline placebo control. The concentration of anti-TIM-3 mAb used in the study is also outlined. (FIG. 7B-C) Longitudinal BLI revealed significantly decreased in vivo S. aureus growth in humanized BLT mice treated with sabatolimab (n=9-10, ANOVA, *p<0.05). At day 14 post-surgery, terminal ex vivo CFU on the tibia and internal organs (heart, liver, kidneys, and spleen) revealed that sabatolimab-treated animals exhibited lower disease severity (n=9-10, t-test, *p<0.05).

### DETAILED DESCRIPTION OF THE INVENTION

This disclosure provides a method of reducing the number of S. aureus bacterial cells in a subject and a method of treating or preventing diseases or disorders caused by S. aureus. This disclosure also provides a method of diagnosis or prognosis of a disease or disorder caused by S. aureus.

Immune Checkpoint Blockade Therapy for Treating S. aureus Infections
A. Methods for reducing bacterial load of pathogenic bacterium or a pathogenic fungus, such as S. aureus

In one aspect, the invention provides a method of reducing the number of cells of a pathogenic bacterium or a pathogenic fungus, such as S. aureus bacterial cells in a subject in need thereof. In some embodiments, the method includes reducing S. aureus bacterial load or eradicating the bacteria in the bloodstream or heart of a mammalian subject. In some embodiments, the method includes administering to the subject an effective amount of an inhibitor of an immune checkpoint molecule. In some embodiments, the method includes administering to the subject an additional therapeutical agent, such as an anti-microbial or anti-bacterial agent (e.g., antibiotic).

A bacterial load in the bloodstream or heart of a mammalian subject can routinely be measured via methods known in the art to determine the amount of bacteria in the bloodstream or heart. For example, the bacterial load can be measured by plating a sample from an organism onto an agar plate, incubating the plate, and then quantifying the number of colony-forming units (CFU) on the plate. Such methods are known in the art. Additional suitable methods for determining bacterial load can also be utilized.

With the method, the bacterial load (i.e., the amount of bacteria as measured by colony-forming units) of a subject infected with S. aureus can be reduced by at least 30% (e.g., at least 40%, 50%, 60%, 70%, 80%, or 90%) in subjects treated with an inhibitor of an immune checkpoint molecule, as compared to a subject that also has been infected with S. aureus, but has not been administered the inhibitor. In some embodiments, an inhibitor of an immune checkpoint molecule can be administered as soon as possible after diagnosis of infection with S. aureus, e.g., within hours or days. The duration and amount of the inhibitor of an immune checkpoint molecule to be administered can be readily determined by those of ordinary skill in the art.

As used herein, the term “immune checkpoint molecule” refers to molecules on immune cells, such as T cells, that are important under normal physiological conditions for the maintenance of self-tolerance (or the prevention of autoimmunity) and the protection of host cells and tissue when the immune system responds to a foreign pathogen. Certain immune checkpoint molecules are co-stimulatory molecules that amplify a signal involved in the T cell response to antigen, while certain immune checkpoint molecules are inhibitory molecules (e.g., CTLA-4 or PD-1) that reduce a signal involved in the T cell response to antigen.

As used herein, the term “inhibitory immune checkpoint molecule” includes in particular inhibitory immune checkpoint receptors, expressed on immune effector cells such as T cells and capable of mediating a down-modulating or inhibiting immune responses upon engagement by their respective ligands. Examples of “inhibitory immune checkpoint molecules” include in include, without limitation, CTLA-4, PD-1, PD-L1, PD-L2, VISTA, TIM-1, TIM-3, TIM-4, LAG-3, TIGIT, galectin-1, galectin 9, CEACAM-1, CEACAM-5, CD69, CD113, GPR56, VISTA, B7-H3 (CD276), B7-H4 (VTCN1), 2B4 (CD244), SLAMF2 (CD48), GITR, BTLA, HVEM, KIR family receptors, GARP, and PD1H. Inhibitors (e.g., antagonistic (blocking) compositions) that bind to an inhibitory immune checkpoint molecule may also be referred to herein an immune checkpoint inhibitor.

In some embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen-presenting cells. PD-1 is another inhibitory immune checkpoint molecule that is expressed on T cells. PD-1 limits the activity of T cells in peripheral tissues during an inflammatory response. PD-L1 is widely expressed on antigen-presenting cells and other immune cells, and its binding to PD-1 on T cells drives them to apoptosis or into a regulatory phenotype. In some embodiments, the inhibitory immune checkpoint molecule is lymphocyte activation gene 3 (LAG-3), T cell membrane protein 3 (TIM-3). Non-limiting examples of immune checkpoint molecules includes LAG-3, TIM-3, CTLA-4, PD-1, and PD-L1.

As used herein, the term “inhibit” or “inhibitor” refers to an alteration, interference, reduction, down-regulation, blocking, suppression, abrogation, or degradation, directly or indirectly, in the expression, amount, or activity of a target or signaling pathway relative to a control, endogenous or reference target or pathway, or the absence of a target or pathway, wherein the alteration, interference, reduction, down-regulation, blocking, suppression, abrogation or degradation is statistically, biologically, or clinically significant. For example, an “inhibitor” of an immune checkpoint molecule blocks, inactivates, reduces or minimizes the activity of the immune checkpoint molecule by about 5%, 10%, 15%, 20%, 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more. Non-limiting examples of inhibitors includes an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof.

Inhibitors that target immune checkpoint molecules can be used to enhance T cell responses against S. aureus. Small molecule inhibitors for immune checkpoint molecules are described, for example, Smith W M, et al. (Smith W M, et al. Therapeutic targeting of immune checkpoints with small molecule inhibitors. Am J Transl Res 11 (2), 529-541 (2019). PMID: 30899360), the content of which is herein incorporated by reference. For example, non-limiting examples of small molecule inhibitors for LAG-3 includes BMS-986016 (BMS-ONO) and GSK2831781. Non-limiting examples of small molecule inhibitors for TIM-3 includes EPZ005687 and DZNep. Non-limiting examples of small molecule inhibitors for CTLA-4 includes entinostat, panobinostat, ACY-241, and azacytidine. Non-limiting examples of small molecule inhibitors for PD-1/PD-L1 includes BMS-8, BMS-37, BMS-202, BMS-230, BMS-242, BMS-1001, BMS-1166, SB415286, JQ1, and I-BET151.

In some embodiments, an inhibitor of an immune checkpoint molecule is an antibody, e.g., a monoclonal antibody. In some embodiments, the inhibitor includes an anti-LAG-3 antibody, an anti-TIM-3 antibody, an anti-CTLA-4 antibody, an anti-PD-1 antibody, an anti-PD-L1 antibody, an antigen-binding fragment thereof, or a combination thereof. In some embodiments, the inhibitor includes an anti-LAG-3 antibody. In some embodiments, the inhibitor includes a monoclonal anti-LAG-3 antibody. In some embodiments, the inhibitor includes an anti-TIM-3 antibody. In some embodiments, the inhibitor includes a monoclonal anti-TIM-3 antibody. In some embodiments, the inhibitor includes an anti-CTLA-4 antibody. In some embodiments, the inhibitor includes a monoclonal anti-CTLA-4 antibody. In some embodiments, the inhibitor includes an anti-PD-1 antibody. In some embodiments, the inhibitor includes a monoclonal anti-PD-1 antibody. In some embodiments, the inhibitor includes an anti-PD-L1 antibody. In some embodiments, the inhibitor includes a monoclonal anti-PD-L1 antibody. In some embodiments, the inhibitor is a combination of two or more of an anti-LAG-3 antibody, an anti-TIM-3 antibody, an anti-CTLA-4 antibody, an anti-PD-1 antibody, and an anti-PD-L1 antibody. In some embodiments, the inhibitor is a combination of two or more of an anti-LAG-3 monoclonal antibody, an anti-TIM-3 monoclonal antibody, an anti-CTLA-4 monoclonal antibody, an anti-PD-1 monoclonal antibody, and an anti-PD-L1 monoclonal antibody.

In some embodiments, the inhibitor comprises (i) an anti-LAG-3 antibody or an antigen-binding fragment thereof, (ii) a combination of an anti-PD-1 antibody or an antigen-binding fragment thereof and an anti-LAG-3 antibody or an antigen-binding fragment thereof, or (iii) a bispecific antibody that binds PD-1 and LAG-3. In some embodiments, the anti-PD-1 antibody is nivolumab, or the anti-LAG-3 antibody is relatlimab.

In some embodiments, the administered LAG-3 inhibitor is an anti-LAG-3 antibody or an antigen-binding fragment thereof. In some embodiments, the administered LAG-3 inhibitor is BMS 986016, MK-4280 (28G-10), REGN3767, GSK2831781, IMP731 (H5L7BW), BAP050, IMP-701 (LAG-5250), TSR-033, LAG525, BI 754111, Sym022, FS-118, an antigen-binding fragment thereof, or a combination thereof. In some embodiments, the administered anti-LAG-3 antibody is MK-4280, IMP-761, GSK2837781, MGD013, BAP050, or IBI110 IMP321 an antigen-binding fragment thereof, or a combination thereof. In some embodiments, the administered anti-LAG-3 antibody is relatlimab, or an antigen-binding fragment thereof, or a combination thereof. In some embodiments, the administered anti-LAG-3 antibody is a LAG-3 binding protein or an antigen-binding fragment thereof, disclosed in U.S. Pat. Nos. 9,244,059, 10,266,591, 9,908,936, 10,358,495, or 10,188,730; or Intl. Publ. No. WO 2008/132601, WO2010/019570, WO2014/008218, WO2017/219995, WO2014/008218, WO2018/066054, WO2019/141092, WO2017/220555, WO2019/129137, WO2018/069500, WO2018/066054, WO2014/008218, WO2015/138920, WO2017/037203, or WO2018/034227 each of which is herein incorporated by reference it its entirety.

In some embodiments, the administered LAG-3 inhibitor is an antibody or an antigen binding fragment of an antibody that specifically binds to human LAG-3 and cross-competes for binding to human LAG-3 with one or more of the above listed antibodies. For example, in some embodiments, the administered LAG-3 inhibitor is an antibody or an antigen binding fragment of an antibody that specifically binds to human LAG-3 and cross-competes for binding to human LAG-3 with relatlimab. The ability of antibodies and antigen binding fragments to cross-compete for binding to an antigen indicates that these antibodies/fragments bind to the same epitope region of the antigen and sterically hinder the binding of other cross-competing antibodies to that particular epitope region. These cross-competing antibodies/fragments are expected to have functional properties very similar to those of the antibody with which they compete (e.g., relatlimab) by virtue of their binding to the same epitope region of LAG-3. Cross-competing antibodies can be readily identified based on their ability to cross-compete with a reference LAG-3 binding antibody (e.g., relatlimab) in standard LAG-3 binding assays such as surface plasmon resonance (BIACORE®) analysis, ELISA assays or flow cytometry (see, e.g., Intl. Publ. No. WO2013/173223).

In some embodiments, the administered TIM-3 inhibitor is an anti-TIM-3 antibody or an antigen-binding fragment thereof. In some embodiments, the administered TIM-3 inhibitor is MBG-453, TSR-022, TRL-6061, BGBA425, LY-3321367, Sym023, INCAGN-2390, MBS-986258, RO-7121661, BC-3402, SHR-1702, or LY-3415244, or an antigen (TIM-3) binding fragment thereof. In some embodiments, the administered TIM-3 inhibitor is the antibody 13A3, 3G4, 17C3, 17C8, 9F6, 8B9, or 8C4 described in Intl. Publ. No, WO2019046321A1 or an antigen (TIM-3) binding fragment thereof. The disclosure of Intl. Publ. No. WO2019046321A is incorporated herein in its entirety. In some embodiments, the administered anti-TIM-3 antibody is TSR-022 or LY3321367, or an antigen-binding fragment thereof, or a combination thereof. In some embodiments, the administered anti-TIM-3 antibody is relatlimab, or an antigen-binding fragment thereof, or a combination thereof. In some embodiments, the administered anti-TIM-3 antibody is a TIM-3 binding protein or an antigen-binding fragment thereof disclosed in Intl. Publ. No. WO 2011/155607, WO2011/159877, WO2013/006490, WO2015/109931, WO2015/117002, WO 2016/068803, WO2016/068802, WO2016/071448, WO2016/111947, WO2016/144803, WO 2016/161270, WO2017/019897, WO2017/031242, WO2017/055399, WO2017/055404, WO 2017/079112, WO2017/079115, WO2017/079116, WO2018/013818, or WO 2019/0046321, U.S. Pat. Nos. 8,552,156, 8,841,418, or U.S. Pat. No. 9,163,087, or Chinese Appl. No. CN 2010/6632675, or an antigen (TIM-3) binding fragment thereof, each of which is herein incorporated by reference it its entirety.

In some embodiments, the administered TIM-3 inhibitor is an antibody or an antigen binding fragment of an antibody that specifically binds to human TIM-3 and cross-competes for binding to human TIM-3 with one or more of the above listed antibodies. For example, in some embodiments, the administered TIM-3 inhibitor is an antibody or an antigen binding fragment of an antibody that specifically binds to human TIM-3 and cross-competes for binding to human TIM-3 with a reference TIM-3 binding antibody (e.g., 13A3, 3G4, 17C3, 17C8, 9F6, 8B9, or 8C4) in standard TIM-3 binding assays such as surface plasmon resonance (BIACORE®) analysis, ELISA assays or flow cytometry (see, e.g., Intl. Publ. No. WO 2013/173223). In another example, in some embodiments, the administered TIM-3 inhibitor is an antibody or an antigen binding fragment of an antibody that specifically binds to human TIM-3 and cross-competes for binding to human TIM-3 with a reference TIM-3 binding antibody disclosed in WO 2019/0046321 in standard TIM-3 binding assays such as surface plasmon resonance (BIACORE®) analysis, ELISA assays or flow cytometry.

Additional TIM-3 inhibitors that can be used in the methods described herein include MBG-453, TSR-022, TRL-6061, BGBA425, and LY-3321367, as well as antigen binding fragments thereof and any other TIM-3 inhibitors, e.g., antibodies, peptides, small molecules, and bispecific molecules, such as bispecific antibodies (e.g., anti-TIM-3/anti-PD-1 bispecific molecules).

As used herein, TIM-3 inhibitors include, but are not limited to, anti-TIM-3 antibodies, and antigen binding portions thereof, and soluble TIM-3 polypeptides (e.g., TIM-3-Fc fusion protein that is capable of binding to a TIM-3 ligand).

Bispecific antibodies specifically binding to PD-1 and LAG-3 include those described in WO2018185043A1 (Roche), the content of which is herein incorporated by reference.

In some embodiments, the administered CTLA-4 inhibitor is an anti-CTLA-4 antibody or an antigen-binding fragment thereof. In some embodiments, the administered CTLA-4 inhibitor is includes ipilimumab (e.g., YERVOY®), tremelimumab (ticilimumab; CP-675,206), AGEN-1884, ATOR-1015, an antigen-binding fragment thereof, or a combination thereof.

In some embodiments, the administered anti-CTLA-4 antibody is a CTLA-4 binding protein or an antigen-binding fragment thereof disclosed in U.S. Pat. Nos. 5,811,097, 6,682,736, or 7,605,238, Intl. Publ. No. WO2000/32231, WO2000/37504, or WO1997/20574, each of which is herein incorporated by reference it its entirety. In some embodiments, the administered CTLA-4 inhibitor is an antibody or an antigen binding fragment of an antibody that specifically binds to human CTLA-4 and cross-compete for binding to human CTLA-4 with one or more of the above listed antibodies. For example, in some embodiments, the administered CTLA-4 inhibitor is an antibody or an antigen binding fragment of an antibody that specifically binds to human CTLA-4 and cross-competes for binding to human CTLA-4 with a reference CTLA-4 binding antibody (e.g., ipilimumab or tremelimumab) in standard CTLA-4 binding assays such as surface plasmon resonance (BIACORE®) analysis, ELISA assays or flow cytometry (see, e.g., Intl. Publ. No. WO 2013/173223).

In some embodiments, the administered PD-1 inhibitor is an anti-PD-1 antibody or an antigen-binding fragment thereof. In some embodiments, the administered PD-1 inhibitor is includes pembrolizumab (e.g., KEYTRUDA®; MK-3475), pidilizumab (e.g., CT-011), nivolumab (e.g., OPDIVO®; BMS-936558), PDR001, MEDI0680 (AMP-514), TSR-042, REGN2810, JS001, AMP-224 (GSK-2661380), a B7-DC Fc fusion protein), PF-06801591, BGB-A317, BI 754091, JNJ-63723283, tislelizumab, Sym021, MDX-1106, MDX-1106-04, ONO-4538, SHR-1210, an antigen-binding fragment thereof, or a combination thereof. In some embodiments, the administered PD-1 inhibitor is the human antibody 17D8, 2D3, 4H1, 4A11, 7D3 or 5F4 described in U.S. Pat. No. 8,008,449, or an antigen (PD-1) binding fragment thereof.

In some embodiments, the administered anti-PD-1 antibody is a PD-1 binding protein or an antigen-binding fragment thereof disclosed in U.S. Pat. Nos. 8,008,449, 8,354,509, 8,609,089, or 8,747,847, Intl. Publ. No. WO2004/056875, WO2009/114335, WO2006/121168, WO2009/101611, WO2010/027827, WO201/0027423, or WO2011/066342, each of which is herein incorporated by reference it its entirety.

In some embodiments, the administered PD-1 inhibitor is an antibody or an antigen binding fragment of an antibody that specifically binds to human PD-1 and cross-competes for binding to human PD-1 with one or more of the above listed antibodies. For example, in some embodiments, the administered PD-1 inhibitor is an antibody or an antigen binding fragment of an antibody that specifically binds to human PD-1 and cross-compete for binding to human PD-1 with nivolumab (see, e.g., U.S. Pat. Nos. 8,008,449 and 8,779,105; and Intl. Publ. No. WO 2013/173223). The ability of antibodies and antigen binding fragments to cross-compete for binding to an antigen indicates that these antibodies/fragments bind to the same epitope region of the antigen and sterically hinder the binding of other cross-competing antibodies to that particular epitope region. These cross-competing antibodies/fragments are expected to have functional properties very similar to those of the antibody with which they compete (e.g., nivolumab) by virtue of their binding to the same epitope region of PD-1. Cross-competing antibodies can be readily identified based on their ability to cross-compete with a reference PD-1 binding antibody (e.g., nivolumab) in standard PD-1 binding assays such as surface plasmon resonance (BIACORE®) analysis, ELISA assays or flow cytometry (see, e.g., Intl. Publ. No. WO 2013/173223).

In some embodiments, the administered anti-PD-1 antibody is nivolumab (e.g., OPDIVO®). In some embodiments, the administered anti-PD-1 antibody is pembrolizumab (e.g., KEYTRUDA®).

In some embodiments, the administered PD-L1 inhibitor is an anti-PD-L1 antibody or an antigen-binding fragment thereof. In some embodiments, the administered PD-L1 inhibitor is includes atezolizumab (e.g., TECENTRIQ®) RG7446; MPDL3280A; ATEZO®), R05541267), durvalumab (e.g., MEDI4736, IMFINZI®), BMS-936559, avelumab (e.g., BAVENCIOR), LY3300054, CS1001, CX-072 (Proclaim-CX-072), FAZ053, KN035, MDX-1105, W243.55.S70, STI-A1014, YW243.55.570, MSB-0010718C, an antigen-binding fragment thereof, or a combination thereof. In some embodiments, the administered anti-PD-L1 antibody is a PD-L1 binding protein or an antigen-binding fragment thereof disclosed in U.S. Pat. Nos. 7,943,743, 8,217,149, 8,779,108, or 9,324,298; or Intl. Publ. No. WO2013/079174 or WO2016061142, each of which is herein incorporated by reference it its entirety.

In some embodiments, the administered PD-L1 inhibitor is an antibody or an antigen binding fragment of an antibody that specifically binds to human PD-L1 and cross-compete for binding to human PD-L1 with one or more of the above listed antibodies. For example, in some embodiments, the administered PD-L1 inhibitor is an antibody or an antigen binding fragment of an antibody that specifically binds to human PD-L1 and cross-competes for binding to human PD-L1 with a reference PD-L1 binding antibody (e.g., atezolizumab, durvalumab, or avelumab) in standard PD-L1 binding assays such as surface plasmon resonance (BIACORE®) analysis, ELISA assays or flow cytometry (see, e.g., Intl. Publ. No. WO 2013/173223).

In some embodiments, the administered TIGIT inhibitor is an anti-TIGIT antibody or an antigen-binding fragment thereof. In some embodiments, the administered TIGIT inhibitor is BMS-986207, AB154, COM902 (CGEN-15137), or etigilimab (e.g., OMP-313M32), or an antigen (TIGIT) binding fragment thereof. In some embodiments, the administered TIGIT inhibitor is the anti-TIGIT antibody MBSA43, MK-7684, AB154, MTIG7192A, MTIG7192A, or an antigen (TIGIT) binding fragment thereof. In some embodiments, the administered anti-TIGIT antibody is BMS-986207, or an antigen-binding fragment thereof. In some embodiments, the administered anti-TIGIT antibody is OMP-313M32, or an antigen-binding fragment thereof. In some embodiments, the administered anti-TIGIT antibody is MTIG7192A, or an antigen-binding fragment thereof. In some embodiments, the administered anti-TIGIT antibody is a TIGIT binding protein or an antigen-binding fragment thereof disclosed in Intl. Publ. No. WO2016/011264, WO2016/106302, WO2016/191643, or WO2017/053748, each of which is herein incorporated by reference it its entirety.

In some embodiments, the administered TIGIT inhibitor is an antibody or an antigen binding fragment of an antibody that specifically binds to human TIGIT and cross-competes for binding to human TIGIT with one or more of the above listed antibodies. For example, in some embodiments, the administered TIGIT inhibitor is an antibody or an antigen binding fragment of an antibody that specifically binds to human TIGIT and cross-competes for binding to human TIGIT with a reference TIGIT binding antibody (e.g., BMS-986207, OMP-313M32 and MTIG7192A) in standard TIGIT binding assays such as surface plasmon resonance (BIACORE®) analysis, ELISA assays or flow cytometry.

In some embodiments, the administered inhibitor is a bispecific antibody that binds at least one inhibitory immune checkpoint molecule. In some embodiments, the administered bispecific antibody binds 2 different inhibitory immune checkpoint inhibitory molecules. In further embodiments, the administered bispecific antibody binds PD-1 and LAG-3. In further embodiments, the administered bispecific antibody comprises antigen binding fragments of nivolumab and relatlimab

### Antibodies

In some embodiments, the antibodies targeting immune checkpoint molecules described herein may be derived from particular murine heavy and light chain germline sequences and/or comprise particular structural features such as CDR regions comprising particular amino acid sequences. Further provided are methods of making such antibodies and immune-conjugates comprising such antibodies or antigen-binding fragments thereof, and pharmaceutical compositions formulated to contain the antibodies or fragments. Also provided herein are methods of using the antibodies for preventing and treating S. aureus infections. Accordingly, the antibodies described herein can be used in a treatment in a wide variety of therapeutic applications, including, for example, treating bacteremia, osteomyelitis, and sepsis.

Unless otherwise indicated or clear from the context, the term “antibody” as used herein includes whole antibodies and any antigen-binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen-binding fragment thereof. Each heavy chain is comprised of a heavy chain variable region (abbreviated herein as V_{H}) and a heavy chain constant region. In certain naturally occurring IgG, IgD, and IgA antibodies, the heavy chain constant region is comprised of three domains, CH1, CH2, and CH3. In certain naturally occurring antibodies, each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four framework regions (FRs), arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Antibodies typically bind specifically to their cognate antigen with high affinity, reflected by a dissociation constant (K_{D}) of 10⁻⁷ to 10⁻¹¹ M or less. Any K_{D} greater than about 10⁻⁶ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen. By way of example, an antibody that binds specifically to human immune checkpoint molecules might also cross-react with immune checkpoint molecules from certain non-human primate species (e.g., cynomolgus monkey), but might not cross-react with immune checkpoint molecules from other species, or with an antigen other than immune checkpoint molecules.

Unless otherwise indicated, an immunoglobulin may be from any of the commonly known isotypes, including but not limited to IgA, secretory IgA, IgG, and IgM. The IgG isotype is divided into subclasses in certain species: IgG1, IgG2, IgG3, and IgG4 in humans, and IgG1, IgG2a, IgG2b, and IgG3 in mice. Immunoglobulins, e.g., human IgG1, exist in several allotypes, which differ from each other in at most a few amino acids. Unless otherwise indicated, “antibody” may include, by way of example, monoclonal and polyclonal antibodies; chimeric and humanized antibodies; human and non-human antibodies; wholly synthetic antibodies; and single chain antibodies.

The term “antigen-binding portion” or “antigen-binding fragment” of an antibody, as used herein, refers to one or more fragments of an antibody that retain the ability to specifically bind to an antigen. Examples of binding fragments encompassed within the term “antigen-binding portion/fragment” of an antibody include (i) a Fab fragment—a monovalent fragment consisting of the V_{L}, V_{H}, C_{L} and CH1 domains; (ii) a F(ab′)2 fragment—a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, and (v) a dAb fragment (Ward et al. (1989) Nature 341:544-546) consisting of a V_{H} domain. An isolated complementarity determining region (CDR), or a combination of two or more isolated CDRs joined by a synthetic linker, may comprise an antigen-binding domain of an antibody that is able to bind antigen.

Single chain antibody constructs are also included in the invention. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird et al. Science 242, 423-426 (1988); and Huston et al. Proc Natl Acad Sci. (USA) 85, 5879-5883 (1988)). Such single chain antibodies are also intended to be encompassed within the term “antigen-binding portion/fragment” of an antibody. These and other potential constructs are described by Chan & Carter Nat Rev Immunol 10, 301 (2010). These antibody fragments are routinely obtained using conventional techniques known to those with skill in the art, and the fragments are screened for utility in the same manner as intact antibodies. Antigen-binding portions/fragments can be produced by recombinant DNA techniques, or by enzymatic or chemical cleavage of intact immunoglobulins.

The term “monoclonal antibody,” as used herein, refers to an antibody that displays a single binding specificity and affinity for a particular epitope or a composition of antibodies in which all antibodies display a single binding specificity and affinity for a particular epitope. Typically such monoclonal antibodies will be derived from a single cell or nucleic acid encoding the antibody, and will be propagated without intentionally introducing any sequence alterations. Accordingly, the term “human monoclonal antibody” refers to a monoclonal antibody that has variable and optional constant regions derived from human germline immunoglobulin sequences. In one embodiment, human monoclonal antibodies are produced by a hybridoma, for example, obtained by fusing a B cell obtained from a transgenic or transchromosomal non-human animal (e.g., a transgenic mouse having a genome comprising a human heavy chain transgene and a light chain transgene), to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations that occur, for example, during antibody maturation. As known in the art (see, e.g., Lonberg, Nature Biotech 23(9, 1117-1125 (2005)), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid sequences that encode the light chain and heavy Chain immunoglobulin polypeptides in response to an antigen may not be identical to the original germline sequences, but instead will be substantially identical or similar (i.e., have at least 80% identity).

A “human” antibody refers to an antibody having variable regions in which both the framework and CDR regions are derived from human germline immunoglobulin sequences. Furthermore, if the antibody contains a constant region, the constant region also is derived from human germline immunoglobulin sequences. Human antibodies of the present invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody, e.g., a mouse antibody, are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species, and the constant regions are derived from another species, such as an antibody in which the variable regions are derived from a mouse antibody, and the constant regions are derived from a human antibody. A “hybrid” antibody refers to an antibody having heavy and light chains of different types, such as a mouse (parental) heavy chain and a humanized light chain, or vice versa.

An “isolated antibody,” as used herein, refers to an antibody that is substantially free of other antibodies having different antigenic specificities (e.g., an isolated antibody that specifically binds to an immune checkpoint molecule (e.g., PD-1) is substantially free of antibodies that specifically bind antigens other than the immune checkpoint molecule). An isolated antibody that specifically binds to an epitope of immune checkpoint molecules may, however, have cross-reactivity to other immune checkpoint molecules from different species.

The term “epitope” or “antigenic determinant” refers to a site on an antigen (e.g., huPD-1) to which an immunoglobulin or antibody specifically binds. Epitopes within protein antigens can be formed both from contiguous amino acids (usually a linear epitope) or noncontiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14, or 15 amino acids in a unique spatial conformation. The term “epitope mapping” refers to the process of identification of the molecular determinants on the antigen involved in antibody-antigen recognition. Methods for routinely determining what epitopes are bound by a given antibody are known in the art and include, for example, immunoblotting and immunoprecipitation assays, wherein overlapping or contiguous peptides from (are tested for reactivity with a given antibody (e.g., anti-PD-1 antibody); x-ray crystallography; 2-dimensional nuclear magnetic resonance; yeast display; and HDX-MS (see, e.g., Epitope Mapping Protocols in Methods in Molecular Biology, Vol. 66, G. E. Morris, Ed. (1996)).

Also provided are “conservative sequence modifications” to the antibody sequence provided herein, i.e. nucleotide and amino acid sequence modifications that do not abrogate the binding of the antibody encoded by the nucleotide sequence or containing the amino acid sequence, to the antigen. For example, modifications can be routinely introduced by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an antibody may be replaced with another amino acid residue from the same side chain family. Methods of routinely identifying nucleotide and amino acid conservative substitutions that do not eliminate antigen binding are known in the art. See, e.g., Brummell et al. Biochem 32, 1180-1187 (1993); Kobayashi et al. Protein Eng 12(10), 879-884 (1999); and Burks et al. Pro. Natl Acad Sci (USA) 94, 412-417 (1997).

### Antibody Generation

Various antibodies of the present invention can routinely be produced using a variety of known techniques, such as the standard somatic cell hybridization technique described by Kohler and Milstein, Nature 256, 495-497 (1975). Although somatic cell hybridization procedures are preferred, in principle, other techniques for producing monoclonal antibodies also can be employed, e.g., viral or oncogenic transformation of B lymphocytes, phage display technique using libraries of human antibody genes.

The preferred animal system for preparing hybridomas is the murine system. Hybridoma production in the mouse is a well-established procedure. Immunization protocols and techniques for routine isolation of immunized splenocytes for fusion are known in the art. Fusion partners (e.g., murine myeloma cells) and fusion procedures are also known.

To generate hybridomas producing monoclonal antibodies described herein, splenocytes and/or lymph node cells from mice immunized with an immune checkpoint molecule or an immunogenic fragment therefore can be isolated and fused to an appropriate immortalized cell line, such as a mouse myeloma cell line. The resulting hybridomas can be screened for the production of antigen-specific antibodies. For example, single cell suspensions of splenic lymphocytes from immunized mice can be fused to Sp2/0 nonsecreting mouse myeloma cells (ATCC, CRL 1581) with 50% PEG. Cells can be plated at approximately 2×10⁵ in a flat bottom microtiter plate, followed by a two-week incubation in a selective medium containing 10% fetal Clone Serum, 18% “653” conditioned media, 5% origen (IGEN), 4 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1X HAT (Sigma). After approximately two weeks, cells can be cultured in a medium in which the HAT is replaced with HT. Individual wells can then be screened by ELISA for human monoclonal IgM and IgG antibodies. Once extensive hybridoma growth occurs, a medium can be observed usually after 10-14 days. The antibody-secreting hybridomas can be replated and screened again, and if necessary, the monoclonal antibodies can be subcloned at least twice by limiting dilution. The stable subclones can then be cultured in vitro to generate small amounts of antibodies in a tissue culture medium for characterization.

To purify monoclonal antibodies, selected hybridomas can be grown in two-liter spinner-flasks for monoclonal antibody purification. Supernatants can be filtered and concentrated before affinity chromatography with protein A-sepharose. Eluted IgG can be checked by gel electrophoresis and high performance liquid chromatography to ensure purity. The buffer solution can be exchanged into PBS, and the concentration can be determined by OD280 using an extinction coefficient of 1.43. The monoclonal antibodies can be aliquoted and stored at −80° C.

Chimeric or humanized antibodies can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can routinely be linked to human constant regions using methods known in the art (see, e.g., U.S. Pat. No. 4,816,567 to Cabilly et al.). To create a humanized antibody, the murine CDR regions can be inserted into a human framework using routine methods known in the art (see, e.g., U.S. Pat. No. 5,225,539 to Winter, and U.S. Pat. Nos. 5,530,101; 5,585,089; 5,693,762 and 6,180,370 to Queen et al.).

Human monoclonal antibodies directed against immune checkpoint molecules can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.” See, e.g., Lonberg, et al. Nature 368, 6474 (1994): 856-859; Lonberg, N. Handbook of Experimental Pharmacology 113, 49-101 (1994); Lonberg, N. and Huszar, D. Intern Rev Immunol 13, 65-93 (1995), and Harding, F. and Lonberg, N. Ann NY Acad Sci 764, 536-546 (1995)). The preparation and use of HuMab mice, and the genomic modifications carried by such mice, are further described in Taylor, L. et al. Nucleic Acids Research 20, 6287-6295 (1992); Chen, J. et al. International Immunology 5, 647-656 (1993); Tuaillon et al. Proc Natl Acad Sci (USA) 90, 3720-3724 (1993); Choi et al. Nature Genetics 4, 117-123 (1993); Chen, J. et al. EMBO J 12, 821-830 (1993); Tuaillon et al. J Immunol 152, 2912-2920 (1994); Taylor, L. et al. International Immunology 6, 579-591 (1994); and Fishwild, D. et al. Nature Biotechnology 14, 845-851 (1996), the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; Intl. Publ. Nos. WO 92/03918, WO 93/12227, WO 94/25585, WO 97/13852, WO 98/24884 and WO 99/45962, all to Lonberg and Kay; and Intl. Publ. No. WO 01/14424 to Korman et al. In some embodiments, antibodies described herein are raised using a mouse that carries human immunoglobulin sequences on transgenes and trans chromosomes, such as a mouse that carries a human heavy chain transgene and a human light chain trans chromosome. Such mice, referred to herein as “KM mice,” are described in detail in Intl. Publ. No. WO 02/43478 to Ishida et al. Still further, alternative transgenic animal systems expressing human immunoglobulin genes are available in the art and can be used to raise anti-immune checkpoint molecule (e.g., anti-PD-1) antibodies described herein. For example, an alternative transgenic system referred to as the Xenomouse (Abgenix, Inc.) can be used; such mice are described in, for example, U.S. Pat. Nos. 5,939,598; 6,075,181; 6,114,598; 6,150,584 and 6,162,963 to Kucherlapati et al.

Alternative transchromosomic animal systems expressing human immunoglobulin genes are available in the art. They can be used to raise antibodies described herein. For example, mice carrying both a human heavy chain transchromosome and a human light chain transchromosome, referred to as “TC mice” can be used; such mice are described in Tomizuka et al. Proc Natl Acad Sci (USA) 97, 722-727 (2000). Furthermore, cows carrying human heavy and light chain trans chromosomes have been described in the art (Kuroiwa et al. Nature Biotechnology 20, 889-894 (2002)) and can be used to raise antibodies described herein.

Additional mouse systems described in the art for raising human antibodies include (i) the VELOCIMMUNE® mouse (Regeneron Pharmaceuticals, Inc.), in which the endogenous mouse heavy and light chain variable regions have been replaced, via homologous recombination, with human heavy and light chain variable regions, operatively linked to the endogenous mouse constant regions, such that chimeric antibodies (human V/mouse C) are raised in the mice, and then subsequently converted to fully human antibodies using standard recombinant DNA techniques; and (ii) the MeMo® mouse (Merus Biopharmaceuticals, Inc.), in which the mouse contains unrearranged human heavy chain variable regions but a single rearranged human common light chain variable region. Such mice, and use thereof to raise antibodies, are described in, for example, Intl. Publ. No. WO2009/15777, WO 2011/072204, WO 2011/097603, WO 2011/163311, WO 2011/163314, and WO 2012/148873, and U.S. Publ. No. US 2010/0069614.

Human monoclonal antibodies described herein can also be prepared using phage display methods for screening libraries of human immunoglobulin genes. Such phage display methods for isolating human antibodies are established in the art. See, e.g., U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies described herein can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

Antibodies of the present invention can routinely be recombinantly produced in a host cell transfection using, for example, a combination of recombinant DNA techniques and gene transfection methods, as is known in the art.

For example, to express antibodies or antibody fragments thereof, DNAs encoding partial or full-length light and heavy chains, can be obtained by standard molecular biology techniques (e.g., PCR amplification or cDNA cloning using a hybridoma that expresses the antibody of interest), and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vectors, or both genes can be inserted into the same expression vector. The antibody genes are inserted into the expression vector(s) by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the V_{H} segment is operatively linked to the C_{H} segment(s) within the vector and the V_{L} segment is operatively linked to the C_{L} segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, recombinant expression vectors may carry regulatory sequences that control the expression of the antibody chain genes in a host cell. The term “regulatory sequence” is intended to include promoters, enhancers, and other expression control elements (e.g., polyadenylation signals) that control the transcription or translation of the antibody chain genes. Such regulatory sequences are described, for example, in Goeddel (Gene Expression Technology. Methods in Enzymology 185, Academic Press, San Diego, CA (1990)). It will be appreciated by those skilled in the art that the design of the expression vector, including the selection of regulatory sequences, may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP) and polyoma. Alternatively, nonviral regulatory sequences may be used, such as the ubiquitin promoter or β-globin promoter. Still, regulatory elements composed of sequences from different sources, such as the SRα promoter system, which contains sequences from the SV40 early promoter and the long terminal repeat of human T cell leukemia virus type 1 (Takebe, Y. et al. Mol Cell Biol 8, 466-472 (1988)).

In addition to the antibody chain genes and regulatory sequences, recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665, and 5,179,017, all by Axel et al.). For example, typically, the selectable marker gene confers resistance to drugs, such as G418, hygromycin, or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies described herein in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibodies (Boss, M. A. and Wood, C. R. Immunology Today 6, 12-13 (1985)). Antibodies of the present invention can also be produced in glycoengineered strains of the yeast Pichia pastoris. Li et al. Nat Biotechnol 24, 210-215 (2006).

Preferred mammalian host cells for expressing the recombinant antibodies described herein include Chinese Hamster Ovary (CHO cells) (including dhfr-CHO cells, described in Urlaub and Chasin, Proc Natl Acad Sci (USA) 77, 4216-4220 (1980), used with a DHFR selectable marker, e.g., as described in R. J. Kaufman and P. A. Sharp, Mol Biol 159, 601-621 (1982)), NSO myeloma cells, COS cells, and SP2 cells. In particular, for use with NSO myeloma cells, another preferred expression system is the GS gene expression system disclosed in WO 1987/04462, WO1989/01036, and EP338,841. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

The N- and C-termini of antibody polypeptide chains of the present invention may differ from the expected sequence due to commonly observed post-translational modifications. For example, C-terminal lysine residues are often missing from antibody heavy chains. Dick et al. Biotechnol Bioeng 100, 1132 (2008). N-terminal glutamine residues, and to a lesser extent glutamate residues, are frequently converted to pyroglutamate residues on both light and heavy chains of therapeutic antibodies. Dick et al. Biotechnol Bioeng 97:544 (2007); Liu et al. J Biol Chem 286, 11211 (2011).

### Compositions

In some embodiments, an inhibitor of immune checkpoint molecules for use in the disclosed methods may be provided in a pharmaceutical composition that is optionally formulated with a pharmaceutically acceptable carrier. The composition may optionally contain one or more additional pharmaceutically active ingredients, such as another antibody or a therapeutic agent. The pharmaceutical compositions also can be administered in a combination therapy with, for example, an antibiotic, or a vaccine, etc. In some embodiments, a composition includes an antibody at a concentration of at least 1 mg/ml, 5 mg/ml, 10 mg/ml, 50 mg/ml, 100 mg/ml, 150 mg/ml, 200 mg/ml, 1-300 mg/ml, or 100-300 mg/ml.

In some embodiments, therapeutic compositions disclosed herein can include other compounds, drugs, and/or agents used for the treatment of infections and/or associated symptoms. Such compounds, drugs, and/or agents can include, for example, antibiotics.

Accordingly, any of the pharmaceutical compositions provided herein can further include one or more additional anti-microbial agents. Non-limiting examples of such antimicrobial agents include: linezolid, erythromycin, mupirocin, ertapenem, doripenem, imipenem, cilastatin, meropenem, cefadroxil, cefazolin, cefalotin, cefalothin, cephalexin, ceflacor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, ceftaroline fosamil, ceftobiprole, teicoplanin, vancomycin, televancin, emifloxac, clindamycin, lincomycin, daptomycin, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, penicillin G, temocillin, ticarcillin, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sufamethizole, sulfamethoxazole, sulfanilamide, sulfasalazine, sulfisoxazole, trimethoprim-sulfamethoxazole, sulfonamidochrysoidine, demeclocycline, doxycycline, minocycline, oxytetracycline, and tetracycline.

As used herein, “pharmaceutically acceptable carrier” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal, or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

The pharmaceutical compounds described herein may include one or more pharmaceutically acceptable salts. A “pharmaceutically acceptable salt” refers to a salt that retains the desired biological activity of the parent compound and does not impart any undesired toxicological effects (see, e.g., Berge, S. M., et al. J Pharm Sci 66, 1-19 (1977)). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids, such as aliphatic mono- and dicarboxylic acids, phenyl-substituted alkanoic acids, alkanoic hydroxy acids, aromatic acids, aliphatic and aromatic sulfonic acids and the like. Base addition salts include those derived from alkaline earth metals, such as sodium, potassium, magnesium, calcium, and the like, as well as from nontoxic organic amines, such as N,N′-dibenzyl ethylenediamine, N-methylglucamine, chloroprocaine, choline, diethanolamine, ethylenediamine, procaine and the like.

In some embodiments, the pharmaceutical composition can be in the form of sterile aqueous solutions or dispersions. It can also be formulated in a microemulsion, liposome, or other ordered structure suitable for high drug concentration.

In some embodiments, an inhibitor of inhibitory immune checkpoint molecule described herein can be administered as a sustained release formulation, in which case less frequent administration is required. Dosage and frequency vary depending on the half-life of the inhibitor in the patient. For example, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. In therapeutic applications, a relatively high dosage at relatively short intervals is sometimes required until progression of the disease is reduced or terminated, or until the patient shows partial or complete amelioration of symptoms of the disease. Thereafter, the patient can be administered a prophylactic regime.

The amount of active ingredient which can be combined with a carrier material to produce a single dosage form will vary depending upon the subject being treated and the particular mode of administration and will generally be that amount of the composition which produces a therapeutic effect. Generally, out of one hundred percent, this amount will range from about 0.01% to about 99% of the active ingredient, preferably from about 0.1% to about 70%, most preferably from about 1% to about 30% of the active ingredient in combination with a pharmaceutically acceptable carrier.

Dosage regimens are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit contains a predetermined quantity of active compound calculated to produce the desired therapeutic effect in association with the required pharmaceutical carrier. Alternatively, an antibody can be administered as a sustained release formulation, in which case less frequent administration is required. For administration of the antibody, the dosage ranges from about 0.0001 to 100 mg/kg, and more usually 0.01 to 5 mg/kg of the host body weight. For example, dosages can be 0.3 mg/kg body weight, 1 mg/kg body weight, 3 mg/kg body weight, 5 mg/kg body weight, or 10 mg/kg body weight or within the range of 1-10 mg/kg. An exemplary treatment regime entails administration once per week, once every two weeks, once every three weeks, once every four weeks, once a month, once every 3 months, or once every three to 6 months. In some embodiments, dosage regimens for an antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks. In some methods, dosage is adjusted to achieve a plasma antibody concentration of about 1-1000 μg/ml, and in some methods, about 25-300 μg/ml. A “therapeutically effective dosage” of an inhibitor may result in a decrease in severity of disease symptoms, an increase in frequency and duration of disease symptom-free periods, or a prevention of impairment or disability due to the disease affliction.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., J. R. Robinson, ed., Marcel Dekker, Inc., New York, 1978.

The pharmaceutical composition can be administered via medical devices such as needleless hypodermic injection devices (e.g., U.S. Pat. Nos. U.S. Pat. Nos. 5,399,163; 5,383,851; 5,312,335; 5,064,413; 4,941,880; 4,790,824; and 4,596,556); micro-infusion pumps (U.S. Pat. No. U.S. Pat. No. 4,487,603); transdermal devices (U.S. Pat. No. U.S. Pat. No. 4,486,194); (infusion apparatus (e.g., U.S. Pat. Nos. 4,447,233 and 4,447,224); and osmotic devices (e.g., U.S. Pat. Nos. 4,439,196 and 4,475,196). The disclosures of these patents are incorporated herein by reference.

In some embodiments, inhibitors of immune checkpoint molecules described herein can be formulated to ensure proper distribution in vivo. For example, they can be formulated in liposomes, which may additionally comprise targeting moieties to enhance selective transport to specific cells or organs. See, e.g. U.S. Pat. Nos. U.S. Pat. Nos. 4,522,811; 5,374,548; 5,416,016; and 5,399,331; V. V. Ranade, J Clin Pharmacol 29, 685-694 (1989); Umezawa et al. Biochem Biophys Res Commun 153, 1038-1044 (1988); Bloeman et al. FEBS Lett 357, 140-144 (1995); M. Owais et al. Antimicrob Agents Chemother 39, 180 (1995); Briscoe et al. Am J Physiol 1233:134 (1995); Schreier et al. J Biol Chem 269, 9090-9098 (1994); Keinanen and Laukkanen FEBS Lett 346, 123-126 (1994); and Killion and Fidler Immunomethods 4, 273-279 (1994).

B. Methods for Treating Disease or Disorders Caused by Pathogenic Bacterium or a Pathogenic Fungus, Such as S. aureus

In another aspect, this disclosure also provides a method of preventing, treating or ameliorating a disease or disorder caused by a pathogenic bacterium or a pathogenic fungus, such as S. aureus in a subject in need thereof. In some embodiments, the method comprises administering to the subject a therapeutically effective amount of an inhibitor of an immune checkpoint molecule.

In some embodiments, the immune checkpoint molecule is selected from LAG-3, TIM-3, CTLA-4, PD-1, and PD-L1.

In some embodiments, the inhibitor includes an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof.

In some embodiments, the inhibitor includes an anti-LAG-3 antibody, an anti-TIM-3 antibody, an anti-CTLA-4 antibody, an anti-PD-1 antibody, an anti-PD-L1 antibody, or a combination thereof. In some embodiments, the inhibitor includes an anti-LAG-3 antibody.

In some embodiments, the inhibitor comprises (i) an anti-LAG-3 antibody or an antigen-binding fragment thereof, (ii) a combination of an anti-PD-1 antibody or an antigen-binding fragment thereof and an anti-LAG-3 antibody or an antigen-binding fragment thereof, or (iii) a bispecific antibody that binds PD-1 and LAG-3. In some embodiments, the anti-PD-1 antibody is nivolumab, or the anti-LAG-3 antibody is relatlimab.

In some embodiments, the disease or disorder includes an infection of S. aureus. In some embodiments, the infection is a bone infection, such as osteomyelitis. In some embodiments, the infection is a prosthetic joint infection, a fracture related infection, a diabetic foot infection, a hematogenous osteomyelitis, or a spine infection.

In some embodiments, the infection is bacteremia. In some embodiments, the disease or disorder includes S. aureus-associated sepsis.

In some embodiments, the method further includes selecting a subject having a chronic or acute disease or disorder caused by S. aureus. In some embodiments, the subject is suffering from osteomyelitis. As used herein, the term “subject suffering from osteomyelitis” refers to a subject that has one or more symptoms of osteomyelitis (e.g., including but not limited to, pain in the bone, bone tenderness, and swelling or warmth) or a positive diagnosis based on one or more diagnostic tests (e.g., including but not limited to, a bone scan, blood culture, or culture of the infectious lesion). As used herein, the term “subject suffering from osteomyelitis at a particular site of infection” refers to a “subject suffering from osteomyelitis” wherein the osteomyelitis has been identified as being in a particular bone or region of bone or in several particular bones or regions of bones.

S. aureus Infections

In another aspect, the invention provides a method of treating a subject having or at risk of having a S. aureus infection and related conditions (e.g., methicillin-resistant S. aureus (MRSA) infection, methicillin-susceptible S. aureus (MSSA), vancomycin resistant S. aureus (VRSA), daptomycin-resistant S. aureus (DRSA), linezolid-resistant S. aureus (LRSA) and vancomycin intermediate-sensitivity S. aureus (VISA), S. aureus bacteremia, S. aureus skin infection, S. aureus mastitis, S. aureus cellulitis or folliculitis, or S. aureus-involved wound infections, abscesses, osteomyelitis, endocarditis, pneumonia, septic shock, food poisoning, or toxic shock syndrome). The method generally includes administering to a subject (e.g., a human being or another mammal such as a bovine, ovine, canine, feline, equine, hircine, leporine, porcine, or avian) in need thereof a therapeutically effective amount of a pharmaceutical composition described herein.

In some examples, the subject has been diagnosed or identified as having or at risk of having an S. aureus infection (e.g., a MRSA infection, a MSSA infection). Some embodiments further include (prior to the administering step) a step of diagnosing, identifying, or selecting a subject having or at risk of having a S. aureus infection (e.g., a MRSA infection, a MSSA infection). In some examples, the S. aureus infection is a nosocomial infection. In some examples, the subject has previously been treated with an antibacterial treatment, and the prior treatment was unsuccessful.

In some embodiments, prior to the administration of the inhibitor of inhibitory immune checkpoint molecule, the subject is determined to have:
(i) an elevated serum titer of soluble TIM-3 compared to a previously measured TIM-3 serum titer for the subject,
(ii) a serum titer of soluble TIM-3 higher than that in healthy control subjects, or
(iii) a serum titer of soluble TIM-3 is at least 2100, 2200, 2300, 2400, or 2500 μg/ml (e.g., as determined, in a method described herein or a method known in the art).

In some embodiments, prior to the administration of the inhibitor of inhibitory immune checkpoint molecule, the subject is determined to have:
(a)(1) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml, and
(b)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml;

In some embodiments, prior to the administration of the inhibitor of inhibitory immune checkpoint molecule, the subject is determined to have:
(i) an elevated serum titer of soluble LAG-3 compared to a previously measured LAG-3 serum titer for the subject,
(ii) a serum titer of soluble LAG-3 higher than that in healthy control subjects, or
(iii) a serum titer of soluble LAG-3 of at least 80000, 85000, 90000, 95000, or 100000 μg/ml (e.g., as determined, in a method described herein or a method known in the art).

In some embodiments, prior to the administration of the inhibitor of inhibitory immune checkpoint molecule, the subject is determined to have:
(a)(1) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml, and
(b)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml.

In some embodiments, prior to the administration of the inhibitor of inhibitory immune checkpoint molecule, the subject is determined to have:
(a)(1) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml, and
(b)(i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml; and/or
(c)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml.

In some embodiments, the subject has been diagnosed or identified as having or at risk of having an S. aureus infection or unfavorable outcome prior to surgery or receiving an implant. In some embodiments, the subject is determined to have or be at risk of having an S. aureus infection or unfavorable outcome within 18 months, 12 months, 9 months, 6 months, 3 months, 1 month, 3 weeks, 2 weeks or 1 week prior to a surgery or receiving an implant.

In some embodiments, prior to the administration of the inhibitor of inhibitory immune checkpoint molecule and prior to surgery or receiving and implant, the subject is determined to have:
(iii) an elevated serum titer of soluble TIM-3 compared to a previously measured TIM-3 serum titer for the subject,
(iv) a serum titer of soluble TIM-3 higher than that in healthy control subjects, or
(iii) a serum titer of soluble TIM-3 is at least 2100, 2200, 2300, 2400, or 2500 μg/ml (e.g., as determined, in a method described herein or a method known in the art).

In some embodiments, prior to the administration of the inhibitor of inhibitory immune checkpoint molecule and prior to surgery or receiving and implant, the subject is determined to have:
(a)(1) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml, and
(b)(i) an elevated serum titer of CXCL13 compared to a previously measured CXCL13 serum titer for the subject,
   (ii) a serum titer of CXCL13 higher than that in healthy control subjects, or
(iii) a serum titer of CXCL13 is at least 50, 55, 60, 65, 70, or 75 μg/ml; In some embodiments, prior to the administration of the inhibitor of inhibitory immune checkpoint molecule and prior to surgery or receiving and implant, the subject is determined to have:
(iii) an elevated serum titer of soluble LAG-3 compared to a previously measured LAG-3 serum titer for the subject,
(iv) a serum titer of soluble LAG-3 higher than that in healthy control subjects, or
(iii) a serum titer of soluble LAG-3 of at least 80000, 85000, 90000, 95000, or 100000 μg/ml (e.g., as determined, in a method described herein or a method known in the art).

In some embodiments, prior to the administration of the inhibitor of inhibitory immune checkpoint molecule and prior to surgery or receiving and implant, the subject is determined to have:
(a)(1) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml, and
(b)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml.

In some embodiments, prior to the administration of the inhibitor of inhibitory immune checkpoint molecule and prior to surgery or receiving and implant, the subject is determined to have:
(a)(1) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml, and
(b)(i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml; and/or
(c)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml.

In some embodiments, the subject having or at risk of having an S. aureus infection has received a surgery within 1 week, 2 weeks, 3 weeks, 1 month, 3 months, 6 months, 9 months, 12 months, 18 months, or 2 years, prior to the administration of the inhibitor of the inhibitory immune checkpoint molecule. In some embodiments, the subject has received surgery within 6 months, 12 months or 18 months, prior to administration of the inhibitor of the inhibitory immune checkpoint molecule.

In some embodiments, the subject having or at risk of having an S. aureus infection has received an implant within 1 week, 2 weeks, 3 weeks, 1 month, 3 months, 6 months, 9 months, 12 months, 18 months, or 2 years, prior to the administration of the inhibitor of the inhibitory immune checkpoint molecule. In some embodiments, the subject has received a implant within 6 months, 12 months or 18 months, prior to administration of the inhibitor of the inhibitory immune checkpoint molecule.

In some embodiments, prior to the administration of the inhibitor of inhibitory immune checkpoint molecule and after surgery or receiving an implant, the subject is determined to have:
(i) an elevated serum titer of soluble TIM-3 compared to a previously measured TIM-3 serum titer for the subject,
(ii) a serum titer of soluble TIM-3 higher than that in healthy control subjects, or
(iii) a serum titer of soluble TIM-3 is at least 2100, 2200, 2300, 2400, or 2500 μg/ml (e.g., as determined, in a method described herein or a method known in the art).

In some embodiments, prior to the administration of the inhibitor of inhibitory immune checkpoint molecule and after surgery or receiving an implant, the subject is determined to have:
(a)(1) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml, and
(b)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml;

In some embodiments, prior to the administration of the inhibitor of inhibitory immune checkpoint molecule and after surgery or receiving an implant, the subject is determined to have:
(i) an elevated serum titer of soluble LAG-3 compared to a previously measured LAG-3 serum titer for the subject,
(ii) a serum titer of soluble LAG-3 higher than that in healthy control subjects, or
(iii) a serum titer of soluble LAG-3 of at least 80000, 85000, 90000, 95000, or 100000 μg/ml (e.g., as determined, in a method described herein or a method known in the art).

In some embodiments, prior to the administration of the inhibitor of inhibitory immune checkpoint molecule and after surgery or receiving an implant, the subject is determined to have:
(a)(1) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml, and
(b)(i) an elevated serum titer of CXCL13 compared to a previously measured CXCL13 serum titer for the subject,
   (ii) a serum titer of CXCL13 higher than that in healthy control subjects, or
   (iii) a serum titer of CXCL13 is at least 50, 55, 60, 65, 70, or 75 μg/ml.

In some embodiments, prior to the administration of the inhibitor of inhibitory immune checkpoint molecule and after surgery or receiving an implant, the subject is determined to have:
(a)(1) an elevated serum titer of soluble TIM-3 compared to a previously measured TIM-3 serum titer for the subject,
   (ii) a serum titer of soluble TIM-3 higher than that in healthy control subjects, or
   (iii) a serum titer of soluble TIM-3 is at least 2100, 2200, 2300, 2400, or 2500 μg/ml, and
(b)(i) an elevated serum titer of soluble LAG-3 compared to a previously measured LAG-3 serum titer for the subject,
   (ii) a serum titer of soluble LAG-3 higher than that in healthy control subjects, or
   (iii) a serum titer of soluble LAG-3 is at least 80000, 85000, 90000, 95000, or 100000 μg/ml; and/or
(c)(i) an elevated serum titer of CXCL13 compared to a previously measured CXCL13 serum titer for the subject,
   (ii) a serum titer of CXCL13 higher than that in healthy control subjects, or
(iii) a serum titer of CXCL13 is at least 50, 55, 60, 65, 70, or 75 μg/ml.

Bone infections, such as osteomyelitis, can be debilitating or even fatal, and are notoriously difficult to treat. For example, osteomyelitis is an acute or chronic bone infection that may be caused by bacteria or fungi. The infection that causes osteomyelitis may start in another part of the body and spread to the bone through the blood. The infection may also spread to a bone from infected skin, muscles, or tendons next to the bone, as in osteomyelitis that occurs under a chronic skin ulcer (sore). Bone infection can also start after bone surgery, especially if the surgery is done after an injury or if metal rods or plates are placed in the bone. In children, the long bones are usually affected. In adults, the feet, spine bones (vertebrae), and hips (pelvis) are most commonly affected.

As used herein, the term “osteomyelitis” refers to an infection or inflammation of the bone. Osteomyelitis infections are generally caused by a pathogenic microorganism (e.g., a bacteria or a fungus). Osteomyelitis includes both acute and chronic (e.g., persistent) bone infections. It includes any inflammation of the bone marrow and adjacent bone. Often, the original site of infection is elsewhere in the body, and spreads to the bone by the blood. The bone may be predisposed to infection due to a recent minor trauma that results in a blood clot or hemostasis. In children, the long bones are usually affected. In adults, the vertebrae, head, and pelvis are most commonly affected. Bacteria or fungi are the usual organisms, but any microbe may be responsible for the infection. Pus is produced within the bone, which may result in a bone abscess. The abscess then deprives the bone of blood supply. Chronic osteomyelitis results when the causative microbes become resistant to antimicrobial agents. This may occur due to development of cellular mechanisms to circumvent the antimicrobial agents, formation of biofilms which allow quiescent organisms to remain untouched by antimicrobial agents, death of bone tissue as a result of the lost blood supply, and other mechanisms. Chronic infection can persist for years with intermittent exacerbations. Risk factors for chronic infection are recent trauma, diabetes, hemodialysis patients, IV drug abuse, and infection with organisms that are more adept at forming biofilms or developing antimicrobial resistance.

Bacteremia, also known as blood poisoning, occurs when S. aureus bacteria enter a mammal's bloodstream, including humans. Persistent fever is one sign of bacteremia. The bacteria can travel to locations deep within the body to produce infections affecting internal organs, such as brain, heart, lungs, bones, and muscles, or surgically implanted devices, such as artificial joints or cardiac pacemakers. One hallmark of S. aureus sepsis is bacterial agglutination and thromboembolic lesion formation, which is measured as bacterial colony-forming units (CFU) in the heart.

### Sepsis

In some embodiments, methods are provided for treating or preventing a pathogenic bacterium or a pathogenic fungus, such as S. aureus-associated sepsis in a mammalian subject or reducing the severity of the sepsis in a mammalian subject. Such methods suitably comprise administering to the subject an effective amount of an inhibitor of the immune checkpoint molecule described herein.

“Sepsis,” as used herein, is a condition characterized by a whole-body inflammatory state that is triggered by infection. The infection may be caused by bacteria (such as S. aureus), viruses or fungi. The body may develop this inflammatory response by the immune system to microbes in the blood, urine, lungs, skin, or other tissues. A lay term for sepsis is blood poisoning, also used to describe septicemia. Septicaemia is a related medical term referring to the presence of pathogenic organisms in the bloodstream, leading to sepsis.

Methods of preventing S. aureus-associated sepsis in a mammalian subject include administering an effective amount of an inhibitor of the immune checkpoint molecule described herein to the subject prior to an infection event. As used herein, “infection event” refers to an event during which the subject is, or could be, exposed to S. aureus infection. Exemplary infection events include, but are not limited to, surgery on any part of the body, including head, mouth, hands, arms, legs, trunk, internal organs (e.g., heart, brain, bowels, kidneys, stomach, lungs, liver, spleen, pancreas, etc.), bones, skin. Surgery provides conditions, such as open surgical wounds and organs, which can readily be infected with S. aureus. Additional infection events include trauma to any part of the body that provides open wounds or otherwise access to the bloodstream via which S. aureus infection could enter the body. Additional infection events include blood transfusions, injections of medications or illegal or legal drugs, needle pricks, tattoo needles, insertion and maintenance of intravenous (IV) lines, insertion and maintenance of surgical drains, and sites of skin breakdown, e.g., bedsores (decubitus ulcers). In embodiments where the methods provide prevention of S. aureus-associated sepsis, the antibody or antigen-binding fragment thereof can be administered at least 1 hour prior to an infection event, e.g., at least 12, 18, 24, 30, 36, or 48 hours prior to the infection event.

The above-described antibody can be used alone or in combination with other therapeutic agents in treating SA infection and related conditions. For example, prosthetic joint infection (PJI) is the bane of elective total joint replacement (TJR) surgery, of which the vast majority is caused by Staphylococcal species. The 1-5% incidence of PJI is known to be a nonrandom event that is largely determined by patient-specific factors. Moreover, ~13% of patients infected with S. aureus become septic and die from multiorgan failure, while others recover with little intervention.

In some embodiments, the subject has received or is about to receive a surgery or an implant. In some embodiments, the surgery is selected from the group consisting of orthopedic surgery, cardiothoracic surgery, plastic surgery, neurosurgery, oral surgery, total joint replacement, open reduction internal fixation (ORIF), debridement for open fracture, spine surgery, median sternotomy, revision total joint, revision ORIF, drainage of soft tissue abscess, or organ transplantation surgery.

In some embodiments, the subject has received a surgery within 1 week, 2 weeks, 3 weeks, 1 month, 3 months, 6 months, 9 months, 12 months, 18 months, or 2 years, prior to administration of the inhibitor of the inhibitory immune checkpoint molecule. In some embodiments, the subject has received a surgery within 6 months, 12 months or 18 months, prior to administration of the inhibitor of the inhibitory immune checkpoint molecule.

In some embodiments, the subject has received or is about to receive an implant. As used herein, the term “implant” refers to any medical device (object) intended for placement in the body of a subject (e.g., a mammal, such as a human) that is not a living tissue. Implants have uses that include orthopedic applications, dental applications, ear, nose, and throat (“ENT”) applications, neurosurgical applications, and cardiovascular applications.

In some embodiments, the subject has received an implant within 1 week, 2 weeks, 3 weeks, 1 month, 3 months, 6 months, 9 months, 12 months, 18 months, or 2 years, prior to administration of the inhibitor of the inhibitory immune checkpoint molecule. In some embodiments, the subject has received an implant within 6 months, 12 months or 18 months, prior to administration of the inhibitor of the inhibitory immune checkpoint molecule.

In some embodiments, the subject has received an implant associated with an infection. In some embodiments, the implant contains a biofilm.

In some embodiments, the subject has received an implant and the subject has a bone infection. In further embodiments, the subject has received an implant and the subject has osteiomyelitis.

In some embodiments, the implant is an orthopedic implant. As used herein the term “orthopedic implant” refers to an implant which replaces bone or provides fixation to bone, replaces articulating surfaces of a joint, provides abutment for a prosthetic, or combinations thereof.

Implants can include any combination of artificial materials, combinations selected because of the particular characteristics of the components. For example, a hip implant can include a combination of a metallic shaft to bear the weight, a ceramic artificial joint and a polymeric glue to affix the structure to the surrounding bone. Implants can reside wholly in the body or partly in the body and partly outside the body. Implants can be intended for short-term or long-term residence where they are positioned. Implants can be made of a variety of biocompatible materials, including: metals, ceramics, polymers, gels and fluids not normally found within the human body. Examples of polymers useful in fabricating medical devices/implants include such polymers as silicones, rubbers, latex, plastics, thermoplastics, polyanhydrides, polyesters, polyorthoesters, polyamides, polyacrylo-nitrile, polyurethanes, polyethylene, polytetrafluoroethylene, polyethylenetetraphthalate, polyphazenes, and fluoroplastics. Medical devices can also be fabricated using certain naturally-occurring materials or treated naturally-occurring materials.

To reduce the severity of S. aureus-associated sepsis in a mammalian subject, one can administer an effective amount of an inhibitor of the immune checkpoint molecule described herein to a subject that is exhibiting symptoms of S. aureus-associated sepsis. Such symptoms can include, for example, chills, confusion or delirium, fever or low body temperature (hypothermia), light-headedness due to low blood pressure, rapid heartbeat, shaking, skin rash, and warm skin.

As used herein, “reducing the severity” of sepsis refers to reducing the symptoms that a subject that has acquired S. aureus-associated sepsis is exhibiting. In some embodiments, the symptoms are reduced by at least 30% (e.g., at least 40%, 50%, 60%, 70%, 80%, or 90%) as compared to the symptoms that a subject that also has acquired S. aureus-associated sepsis is exhibiting, but the subject has not been administered the antibody or antigen-binding fragment thereof.

An antibody or antigen-binding fragment thereof described herein can be administered at a suitable dosage and dosage regimen, and such dosage and dosage regimen can depend on the disease or condition. An effective dosage can be identified by determining whether a dosage and dosage regimen gives rise to a therapeutic effect or therapeutic end-point (e.g., prevention). Dosing of the inhibitor of the immune checkpoint molecule can be provided in a single administration, or over multiple administrations spaced according to desired effects and other clinical considerations.

Exemplary methods by which the antibody or antigen-binding fragment thereof can be administered to the subject in any of the various methods described herein include, but are not limited to, intravenous (IV), intratumoral (IT), intralesional (IL), aerosol, percutaneous, endoscopic, topical, intramuscular (IM), intradermal (ID), intraocular (TO), intraperitoneal (IP), transdermal (TD), intranasal (IN), intracerebral (IC), intraorgan (e.g., intrahepatic), slow release implant, or subcutaneous administration, or via administration using an osmotic or mechanical pump.

### Combination Therapy

The compositions and related methods disclosed herein may also be used in combination with additional therapeutic agents or therapies. Non-limiting examples of therapeutic agents include antibiotics such as streptomycin, ciprofloxacin, doxycycline, gentamycin, chloramphenicol, trimethoprim, sulfamethoxazole, ampicillin, tetracycline, vancomycin, linezolid, teicoplanin, rifampin or various combinations of antibiotics.

Alternatively, the therapy may precede or follow the other agent treatment by intervals ranging from minutes to weeks. In embodiments where the other agents and/or proteins or polynucleotides are administered separately, one can generally ensure that a significant period of time does not expire between the time of each delivery, such that the therapeutic composition would still be able to exert an advantageous combined effect on the subject. In some embodiments, one may administer both modalities within about 12-24 h of each other or within about 6-12 h of each other. In some embodiments, the time period for administration may be extended significantly to several days (2, 3, 4, 5, 6 or 7) or to several weeks (1, 2, 3, 4, 5, 6, 7 or 8) between the respective administrations. Administration of the antibiotics can typically be via any common route. This includes, but is not limited to, oral, nasal, or buccal administration. Alternatively, administration may be by orthotopic, intradermal, subcutaneous, intramuscular, intraperitoneal, intranasal, or intravenous injection.

In some embodiments, the additional therapeutic agent is a second inhibitor of a second immune checkpoint molecule, an antibiotic, or a combination thereof. In some embodiments, the inhibitor and the additional therapeutic agent are contained in the same composition. In some embodiments, the second immune checkpoint molecule is selected from LAG-3, TIM-3, CTLA-4, PD-1, and PD-L1. In some embodiments, the second inhibitor is a second inhibitory immune checkpoint inhibitor, such as an anti-LAG-3 antibody, an anti-TIM-3 antibody, an anti-CTLA-4 antibody, an anti-PD-1 antibody, an anti-PD-L1 antibody, or an antigen-binding fragment thereof, or a combination thereof. In some embodiments, the second inhibitor is a inhibitory immune checkpoint inhibitor, such as an anti-TIGIT antibody, or an antigen-binding fragment thereof, or a combination thereof.

In some embodiments, the second administered inhibitory immune checkpoint inhibitor is a killer cell immunoglobulin-like receptor (KIR) inhibitor. In some embodiments, the KIR inhibitor is an anti-KIR antibody or antigen-binding fragment thereof. In some embodiments, the anti-KIR antibody is lirilumab (1-7F9, BMS-986015, IPH 2101) or IPH4102.

In some embodiments, the second administered inhibitory immune checkpoint inhibitoris a LAG-3 inhibitor. In some embodiments, the second administered inhibitory immune checkpoint inhibitor is an anti-LAG-3 antibody or an antigen-binding fragment thereof. In some embodiments, the administered anti-LAG-3 antibody is BMS 986016, MK-4280 (28G-10), REGN3767, GSK2831781, IMP731 (H5L7BW), BAP050, IMP-701 (LAG-5250), TSR-033, LAG525, BI 754111, Sym022, FS-118, an antigen-binding fragment thereof, or a combination thereof. In some embodiments, the administered anti-LAG-3 antibody is MK-4280, IMP-761, GSK2837781, MGD013, BAP050, or IBI110 IMP321 an antigen-binding fragment thereof, or a combination thereof. In some embodiments, the administered anti-LAG-3 antibody is relatlimab, or an antigen-binding fragment thereof, or a combination thereof. In some embodiments, the administered anti-LAG-3 antibody is a LAG-3 binding protein or an antigen-binding fragment thereof, disclosed in U.S. Pat. Nos. 9,244,059, 10,266,591, 9,908,936, 10,358,495, or 10,188,730; or Intl. Publ. No. WO2008/132601, WO2010/019570, WO2014/008218, WO2014/008218, WO2019/141092, WO2017/220555, WO2017/219995, WO2019/129137, WO2018/069500, WO2018/034227, WO2014/008218, WO2015/138920 or WO2017/037203, each of which is herein incorporated by reference it its entirety

In some embodiments, the second administered inhibitory immune checkpoint inhibitor is a TIGIT inhibitor. In one embodiment, the TIGIT antagonist is an anti-TIGIT antibody or antigen-binding fragment thereof. In some embodiments, the second administered inhibitory immune checkpoint inhibitor is the anti-TIGIT antibody BMS-986207, AB 154, COM902 (CGEN-15137), or OMP-313M32, or an antigen-binding fragment thereof. In some embodiments, the second the second administered inhibitory immune checkpoint inhibitor is the anti-TIGIT antibody BMS-986207, AB154, COM902 (CGEN-15137), or etigilimab (e.g., OMP-313M32), or an antigen (TIGIT) binding fragment thereof. In some embodiments, the second administered inhibitory immune checkpoint inhibitor is the anti-TIGIT antibody MBSA43, MK-7684, AB154, MTIG7192A, MTIG7192A, or an antigen (TIGIT) binding fragment thereof. In some embodiments, the administered anti-TIGIT antibody is BMS-986207, or an antigen-binding fragment thereof. In some embodiments, the administered anti-TIGIT antibody is OMP-313M32, or an antigen-binding fragment thereof. In some embodiments, the second administered anti-TIGIT antibody is MTIG7192A, or an antigen-binding fragment thereof. In some embodiments, the second administered anti-TIGIT antibody is a TIGIT binding protein or an antigen-binding fragment thereof disclosed in Intl. Publ. No. WO2016/011264, WO2016/106302, WO2016/191643, or WO2017/053748, each of which is herein incorporated by reference it its entirety.

In some embodiments, the second administered inhibitory immune checkpoint inhibitor is a TIM-3 inhibitor. In some embodiments, the second administered inhibitory immune checkpoint inhibitor is an anti-TIM-3 antibody or antigen-binding fragment thereof. In some embodiments, the administered anti-TIM-3 antibody is TSR-022 or LY3321367, or an antigen binding fragment thereof. In some embodiments, the second administered anti-TIM-3 antibody is an antigen-binding fragment of TSR-022 or LY3321367, In some embodiments, the administered TIM-3 inhibitor is MBG-453, TRL-6061, BGBA425, Sym023, INCAGN-2390, MBS-986258, RO-7121661, BC-3402, SHR-1702, or LY-3415244, or an antigen (TIM-3) binding fragment thereof. In some embodiments, the administered TIM-3 inhibitor is the antibody 13A3, 3G4, 17C3, 17C8, 9F6, 8B9, or 8C4 described in Intl. Publ. No, WO2019/046321A1 or an antigen (TIM-3) binding fragment thereof. In some embodiments, the administered anti-TIM-3 antibody is TSR-022 or LY3321367, or an antigen-binding fragment thereof, or a combination thereof. In some embodiments, the administered anti-TIM-3 antibody is relatlimab, or an antigen-binding fragment thereof, or a combination thereof. In some embodiments, the administered anti-TIM-3 antibody is a TIM-3 binding protein or an antigen-binding fragment thereof disclosed in Intl. Publ. No. WO 2011/155607, WO2011/159877, WO2013/006490, WO2015/109931, WO2015/117002, WO 2016/068803, WO2016/068802, WO2016/071448, WO2016/111947, WO2016/144803, WO 2016/161270, WO2017/019897, WO2017/031242, WO2017/055399, WO2017/055404, WO 2017/079112, WO2017/079115, WO2017/079116, WO2018/013818, or WO 2019/0046321, U.S. Pat. Nos. 8,552,156, 8,841,418, or U.S. Pat. No. 9,163,087, or Chinese Appl. No. CN 2010/6632675, or an antigen (TIM-3) binding fragment thereof, each of which is herein incorporated by reference it its entirety.

In some embodiments, the second administered inhibitory immune checkpoint inhibitor is a CTLA-4 inhibitor. In some embodiments, the second administered inhibitory immune checkpoint inhibitor is an anti-CTLA-4 antibody or antigen-binding fragment thereof. In some embodiments, the administered anti-CTLA-4 antibody is ipilimumab (e.g., YERVOY®), tremelimumab (ticilimumab; CP-675,206), AGEN-1884, ATOR-1015, an antigen-binding fragment thereof, or a combination thereof. In some embodiments, the second administered inhibitory immune checkpoint inhibitor is a CTLA-4 binding protein or an antigen-binding fragment thereof disclosed in U.S. Pat. Nos. 5,811,097, 6,682,736, 7,605,238, Intl. Publ. No. WO2000/32231, WO2000/37504, and WO1997/20574, each of which is herein incorporated by reference it its entirety. In some embodiments, the second administered inhibitory immune checkpoint inhibitor is a PD-1 inhibitor. In some embodiments, the second administered inhibitory immune checkpoint inhibitor is an anti-PD-1 antibody or antigen-binding fragment thereof. In some embodiments, the administered anti-PD-1 antibody is pembrolizumab (e.g., KEYTRUDA®; MK-3475), pidilizumab (e.g., CT-011), nivolumab (e.g., OPDIVO®; BMS-936558), PDR001, MEDI0680 (AMP-514), TSR-042, REGN2810, JS001, AMP-224 (GSK-2661380), a B7-DC Fc fusion protein), PF-06801591, BGB-A317, BI 754091, JNJ-63723283, tislelizumab, Sym021, MDX-1106, MDX-1106-04, ONO-4538, SHR-1210, an antigen-binding fragment thereof, or a combination thereof. In some embodiments, the administered PD-1 inhibitor is the human antibody 17D8, 2D3, 4H1, 4A11, 7D3 or 5F4 described in U.S. Pat. No. 8,008,449, or an antigen (PD-1) binding fragment thereof. In some embodiments, the administered anti-PD-1 antibody is a PD-1 binding protein or an antigen-binding fragment thereof disclosed in U.S. Pat. Nos. 8,008,449, 8,354,509, 8,609,089, or 8,747,847, Intl. Publ. No. WO2004/056875, WO2009/114335, WO2006/121168, WO2009/101611, WO2010/027827, WO2010/027423, WO2011/066342, or WO2015112900, each of which is herein incorporated by reference it its entirety.

In some embodiments, the administered anti-PD-1 antibody is nivolumab (e.g., OPDIVO®). In some embodiments, the administered anti-PD-1 antibody is pembrolizumab (e.g., KEYTRUDA®). In some embodiments, the second administered inhibitory immune checkpoint inhibitor is nivolumab. In some embodiments, the second administered inhibitory immune checkpoint inhibitor is pembrolizumab.

In some embodiments, the second administered inhibitory immune checkpoint inhibitor is a PD-L1 inhibitor. In some embodiments, the second administered inhibitory immune checkpoint inhibitor is an anti-PD-L1 antibody or antigen-binding fragment thereof. In some embodiments, the administered anti-PD-L1 antibody is atezolizumab (e.g., TECENTRIQ®) RG7446; MPDL3280A; ATEZOR), R05541267), durvalumab (e.g., MEDI4736, IMFINZI®), BMS-936559, avelumab (e.g., BAVENCIOR), LY3300054, CS1001, CX-072 (Proclaim-CX-072), FAZ053, KN035, MDX-1105, W243.55.S70, STI-A1014, YW243.55.570, MSB-0010718C, an antigen-binding fragment thereof, or a combination thereof. In some embodiments, the second administered inhibitory immune checkpoint inhibitor is an anti-PD-L1 antibody or antigen-binding fragment thereof disclosed in U.S. Pat. Nos. 7,943,743, 8,217,149, 8,779,108, or 9,324,298; or Intl. Publ. No. WO2013/079174 or WO2016061142A1, each of which is herein incorporated by reference it its entirety.

In some embodiments, the second administered inhibitory immune checkpoint inhibitor is a CEACAM (e.g., CEACAM-1, CEACAM-3 and/or CEACAM-5) inhibitor. In some embodiments, the administered CEACAM inhibitor is an anti-CEACAM antibody or antigen binding fragment thereof.

In some embodiments, the second administered inhibitory immune checkpoint inhibitor is a CEACAM1 inhibitor. In some embodiments, the administered CEACAM1 inhibitor is an anti-CEACAM1 antibody or antigen binding fragment thereof. In some embodiments, the administered anti-CEACAM1 antibody is CM-24 (MK-6018), or an antigen-binding fragment thereof.

In some embodiments, the second administered inhibitory immune checkpoint inhibitor is a CEA inhibitor. In one embodiment, the administered CEA inhibitor is an anti-CEA antibody or antigen binding fragment thereof. In some embodiments, the anti-CEA antibody is cergutuzumab amunaleukin (RG7813, RO-6895882), RG7802 (RO6958688), an antigen-binding fragment thereof, or a combination thereof.

In some embodiments, the second inhibitory immune checkpoint inhibitor is an Indoleamine 2,3-dioxygenase 1 (IDO1) inhibitor. In another embodiment, the IDO1 inhibitor is indoximod (NLG8189; 1-methyl-D-TRP), epacadostat (INCB-024360, INCB-24360), KHK2455, PF-06840003, navoximod (RG6078, GDC-0919, NLG919), BMS-986205 (F001287), or pyrrolidine-2,5-dione derivatives.

In some embodiments, the second administered inhibitory immune checkpoint inhibitor is a CD80 inhibitor. In some embodiments, the second administered inhibitory immune checkpoint inhibitor is an anti-CD80 antibody or antigen-binding fragment thereof. In some embodiments, the administered anti-CD80 antibody is galiximab, AV 1142742, an antigen-binding fragment thereof, or a combination thereof.

In some embodiments, the second inhibitory immune checkpoint inhibitor is an A2aR inhibitor. In some embodiments, the A2aR inhibitor is a small molecule. In some embodiments, the A2aR inhibitor is CPI-444, PBF-509, istradefylline (KW-6002), preladenant (SCH420814), tozadenant (SYN115), vipadenant (BIIB014), HTL-1071, ST1535, SCH412348, SCH442416, SCH58261, ZM241385, or AZD4635.

In some embodiments, the additional administered therapeutic agent is a chemokine inhibitor. In some embodiments, the chemokine inhibitor is an antibody or antigen binding fragment thereof. In some embodiments, the second administered therapeutic agent is an inhibitor of CXCL13. In some embodiments the agent is an antibody or antigen binding fragment that binds CXCL13.

In some embodiments, the additional administered therapeutic agent is a CD20 inhibitor. In some embodiments, the second administered inhibitory immune checkpoint inhibitor is an anti-CD20 antibody or antigen-binding fragment thereof. In some embodiments, the administered anti-CD20 antibody is rituximab (e.g., RITUXAN®; IDEC-102; IDEC-C2B8), ABP 798, ofatumumab, or obinutuzumab. an antigen-binding fragment thereof, or a combination thereof.

In some embodiments, the additional administered therapeutic agent is a GARP inhibitor. In some embodiments, the second administered inhibitory immune checkpoint inhibitor is an anti-GARP antibody or antigen binding fragment thereof. In some embodiments, the administered anti-GARP antibody is ARGX-115, or an antigen-binding fragment thereof.

In some embodiments, the additional administered therapeutic agent is a CD40 inhibitor. In some embodiments, the CD40 inhibitor is an anti-CD40 antibody or antigen binding fragment thereof. In some embodiments, the administered anti-CD40 antibody is BMS3 h-56, lucatumumab (HCD122 and CHIR-12.12), CHIR-5.9, dacetuzumab (huS2C6, PRO 64553, RG 3636, SGN 14, SGN-40), an antigen-binding fragment thereof, or a combination thereof. In another embodiment, the CD40 inhibitor is a soluble CD40 ligand (CD40-L). In one embodiment, the soluble CD40 ligand is a fusion polypeptide. In one embodiment, the soluble CD40 ligand is a CD40-L/FC2 or a monomeric CD40-L.

In some embodiments, the additional administered therapeutic agent is a CD47 inhibitor. In some embodiments, the administered CD47 inhibitor is an anti-CD47 antibody or antigen binding fragment thereof. In some embodiments, the administered anti-CD47 antibody is HuF9-G4, CC-90002, TTI-621, ALX148, NI-1701, NI-1801, SRF231, Effi-DEM, an antigen-binding fragment thereof, or a combination thereof.

In some embodiments, the additional administered therapeutic agent is a Poliovirus Receptor-Related Immunoglobulin Domain-Containing Protein (PVRIG) inhibitor. In some embodiments, the administered PVRIG inhibitor is an anti-PVRIG antibody or antigen binding fragment thereof. In one embodiment, the administered anti-PVRIG antibody is COM701 (CGEN-15029), or an antigen-binding fragment thereof.

In some embodiments, the additional administered therapeutic agent is a Stimulator of Interferon Genes (STING) inhibitor. In some embodiments, the STING inhibitor is 2′ or 3′-mono-fluoro substituted cyclic-di-nucleotides; 2′3′-di-fluoro substituted mixed linkage 2′,5′-3′,5′ cyclic-di-nucleotides; 2′-fluoro substituted, bis-3′,5′ cyclic-di-nucleotides; 2′,2″-diF-Rp, Rp, bis-3′,5′ cyclic-di-nucleotides; or fluorinated cyclic-di-nucleotides.

In some embodiments, the administered antibiotic has an anti-bacterial activity against S. aureus. Non-limiting examples of antibiotics includes: linezolid, erythromycin, mupirocin, ertapenem, doripenem, imipenem, cilastatin, meropenem, cefadroxil, cefazolin, cefalotin, cefalothin, cephalexin, ceflacor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, ceftaroline fosamil, ceftobiprole, teicoplanin, vancomycin, televancin, clindamycin, lincomycin, daptomycin, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, penicillin G, temocillin, ticarcillin, bacitracin, colistin, polymyxin B, ciprofloxacin, enoxacin, gatifloxacin, gemifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sufamethizole, sulfamethoxazole, sulfanilamide, sulfasalazine, sulfisoxazole, trimethoprim-sulfamethoxazole, sulfonamidochrysoidine, demeclocycline, doxycycline, minocycline, oxytetracycline, and tetracycline.

In some embodiments, the additional administered therapeutic agent is an antibiotic selected from the group consisting of vancomycin, tobramycin, cefazolin, erythromycin, clindamycin, rifampin, gentamycin, fusidic acid, minocycline, co-trimoxazole, clindamycin, linezolid, quinupristin-dalfopristin, daptomycin, tigecycline, dalbavancin, telavancin, oritavancin, ceftobiprole, ceftaroline, iclaprim, the carbapenem CS-023/RO-4908463, and combinations thereof.

In some embodiments, the additional administered therapeutic agent is an immunotherapeutic. In some embodiments, the immunotherapeutic agent is tefibazumab, BSYX-A1 10, Aurexis™, or a combination thereof.

Diagnosis or Prognosis of a Pathogenic Bacterium or a Pathogenic Fungus S. aureus Infections

This disclosure also provides a method of diagnosis or prognosis of a disease or disorder caused by a pathogenic bacterium or a pathogenic fungus, such as S. aureus in a subject in need thereof. In some embodiments, the method includes: (a) determining a level of each of a set of biomarkers in a sample from the subject, wherein the set of biomarkers may include an immune checkpoint molecule or a cytokine; (b) determining a change in the level of each of the set of biomarkers as compared to a reference level for each of the set of biomarkers; and (c) assessing the presence of the disease or disorder or status of the disease or disorder based on the change in the level of each of the set of biomarkers as compared to the reference level for each of the set of biomarkers. In some embodiments, the sample from the subject is a serum sample. In some embodiments, the sample from the subject contains bone marrow cells. In some embodiments, the the sample contains blood cells (e.g., peripheral blood mononucleated cells (PBMCS), neutrophils, metamyelocytes, monocytes, or T cells)). I further embodiments, the sample contains T cells, e.g., Th1/Th17 cells.

In some embodiments, the change in the level of each of one or more biomarkers is an increase in the expression level of each of one or more biomarkers as compared to a reference level. In some embodiments, a subject having an acute S. aureus infection has an increased expression level of each of one or more biomarkers as compared to a subject having a chronic S. aureus infection.

In some embodiments, the set of biomarkers includes one or more of TIM-3, LAG-3, PD-1, CTLA-4, IFNγ, IL-2, TNFα, and IL-17.

In some embodiments, the set of biomarkers comprises TIM-3. In some embodiments, the set of biomarkers comprises TIM-3 and LAG-3. In some embodiments, the set of biomarkers comprises TIM-3 and CTLA-4. In some embodiments, the set of biomarkers comprises TIM-3 and PD-1. In some embodiments, the set of biomarkers comprises TIM-3, LAG-3, and PD-1. In some embodiments, the set of biomarkers comprises TIM-3, LAG-3, and CTLA-4. In some embodiments, the set of biomarkers comprises TIM-3, CTLA-4, and PD-1. In some embodiments, the set of biomarkers comprises TIM-3, LAG-3, CTLA-4, and PD-1. In some embodiments, the set of biomarkers comprises TIM-3, LAG-3, and PD-1. In some embodiments, the set of biomarkers comprises TIM-3, LAG-3, and CTLA-4. In some embodiments, the set of biomarkers comprises TIM-3, CTLA-4, and PD-1. In some embodiments, the set of biomarkers comprises TIM-3, LAG-3, CTLA-4, and PD-1.

In some embodiments, the set of biomarkers comprises TIM-3 and IL-17. In some embodiments, the set of biomarkers comprises TIM-3, LAG-3, and IL-17. In some embodiments, the set of biomarkers comprises TIM-3, CTLA-4, and IL-17. In some embodiments, the set of biomarkers comprises TIM-3, PD-1, and IL-17. In some embodiments, the set of biomarkers comprises TIM-3, LAG-3, PD-1, and IL-17. In some embodiments, the set of biomarkers comprises TIM-3, LAG-3, CTLA-4, and IL-17. In some embodiments, the set of biomarkers comprises TIM-3, CTLA-4, PD-1, and IL-17. In some embodiments, the set of biomarkers comprises TIM-3, LAG-3, CTLA-4, PD-1, and IL-17. In some embodiments, the set of biomarkers includes TIM-3, LAG-3, and CXCL13.

As used herein, the terms “increase,” “elevate,” “elevated,” “enhance,” and “activate” all generally refer to an increase by a statically significant amount as compared to a reference level (e.g., a reference expression level or serum titer level of healthy control subjects). For the avoidance of any doubt, these terms mean an increase of at least 5% (e.g., 5%, 10%, 15%, 20%, 25%, 30%, 35%, 40%, 45%, 50%, 55%, 60%, 65%, 70%, 75%, 80%, 85%, 90%, 95%, 100%) as compared to a reference level, for example, an increase of at least about 10%, at least about 20%, at least about 30%, at least about 40%, at least about 50%, at least about 60%, at least about 70%, at least about 80%, at least about 90% or at least about 100%, as compared to a reference level. In some embodiments, these terms refer to an increase of 10-20%, 10-30%, 10-40%, 10-50%, 10-60%, 10-70%, 10-80%, 10-90%, 10-100%, 10-110%, 10-120%, 10-130%, 10-140%, 10-150%, 10-160%, 10-170%, 10-180%, 10-190%, 10-200%, 10-210%, 10-220%, 10-230%, 10-240%, 10-250%, 10-260%, 10-270%, 10-280%, 10-290%, or 10-300%, as compared to a reference level. In some embodiments, these terms refer to an increase of 10-300%, 20-300%, 30-300%, 40-300%, 50-300%, 60-300%, 70-300%, 80-300%, 90-300%, 100-300%, 110-300%, 120-300%, 130-300%, 140-300%, 150-300%, 160-300%, 170-300%, 180-300%, 190-300%, 200-300%, 210-300%, 220-300%, 230-300%, 240-300%, 250-300%, 260-300%, 270-300%, 280-300%, or 290-300% as compared to a reference level. In some embodiments, these terms refer to an increase of at least 2-fold, at least 3-fold, at least 4-fold, at least 5-fold, at least 10-fold or greater, as compared to a reference level.

The terms “reference level,” “reference value, “predetermined value,” and “predetermined level” are used interchangeably herein. An increased level (e.g., expression level or serum titer level compared to healthy subjects) of a biomarker as compared to a predetermined reference value can be an increase of at least 5% (e.g., 5%, 10%, 15%, 20%, 25%, 30%, 35%, 40%, 45%, 50%, 55%, 60%, 65%, 70%, 75%, 80%, 85%, 90%, 95%, 100%) as compared to a predetermined reference value, for example, an increase of at least about 10%, at least about 20%, at least about 30%, at least about 40%, at least about 50%, at least about 60%, at least about 70%, at least about 80%, at least about 90%, at least about 100%, or any increase from 10% to 100%, as compared to a predetermined reference value; or at least a 2-fold, at least a 3-fold, at least a 4-fold, at least a 5-fold or at least a 10-fold increase, or any increase from 2-fold to 10-fold or greater, as compared to a predetermined reference value.

In some embodiments, an increased level of a biomarker as compared to a predetermined reference value (e.g., a reference value based on healthy control subjects) can be an increase of 10-20%, 10-30%, 10-40%, 10-50%, 10-60%, 10-70%, 10-80%, 10-90%, 10-100%, 10-110%, 10-120%, 10-130%, 10-140%, 10-150%, 10-160%, 10-170%, 10-180%, 10-190%, 10-200%, 10-210%, 10-220%, 10-230%, 10-240%, 10-250%, 10-260%, 10-270%, 10-280%, 10-290%, or 10-300% as compared to a predetermined reference value. In some embodiments, an increased level of a biomarker as compared to a predetermined reference value can be an increase of 10-300%, 20-300%, 30-300%, 40-300%, 50-300%, 60-300%, 70-300%, 80-300%, 90-300%, 100-300%, 110-300%, 120-300%, 130-300%, 140-300%, 150-300%, 160-300%, 170-300%, 180-300%, 190-300%, 200-300%, 210-300%, 220-300%, 230-300%, 240-300%, 250-300%, 260-300%, 270-300%, 280-300%, or 290-300% as compared to a predetermined reference value. In some embodiments, an increased level of a biomarker as compared to a predetermined reference value can be an increase of 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170%, 170-180%, 180-190%, 190-200%, 200-210%, 210-220%, 220-230%, 230-240%, 240-250%, 250-260%, 260-270%, 270-280%, 280-290%, or 290-300% as compared to a predetermined reference value.

As used herein, the term “higher” with reference to a biomarker or biomarker complex measurement refers to a statistically significant and measurable difference in the level of a biomarker or biomarker complex measurement compared to the level of another biomarker or biomarker complex or to a control level (e.g., the level of the biomarker or biomarker complex in healthy control subjects) where the biomarker or biomarker complex measurement is greater than the level of the other biomarker or biomarker complex or the control level. The difference is preferably at least about 10%, or at least about 20%, or at least about 30%, or at least about 40%, or at least about 50%.

The term “reference level, “control level,” “reference sample,” “reference cell,” “reference tissue,” “control sample,” “control cell,” or “control tissue,” as used herein, refers to a level, standard, sample, cell, or tissue that is used for comparison purposes. The terms “reference level” and “control level” are used interchangeably herein. The terms “reference sample,” “reference cell,” “reference tissue,” “control sample,” “control cell,” and “control tissue are used interchangeably herein. In some embodiments, the reference level refers to the expression or titer level of a biomarker or biomarker complex in healthy control subjects.

The methods described herein includes a prognosis of a subject that includes, with respect to determining the severity of S. aureus infections (e.g., sepsis) or determining the subject's risk for subsequent mortality, and risk assessment of the subject with S. aureus infections (e.g., sepsis). In some embodiments, the method includes determining a level (e.g., expression level) of one or more biomarkers (e.g., TIM-3, LAG-3, PD-1, CTLA-4, IFNγ, IL-2, TNFα, IL-17). In some embodiments, the method includes determining a level of TIM-3 (e.g., TIM-3 expression in a PBMC sample, or soluble TIM-3 in serum). In some embodiments, the method includes determining a level of LAG-3 (e.g., LAG-3 expression in a PBMC sample, or soluble LAG-3 in serum). In some embodiments, the method includes determining a level of TIM-3 and CXCL13 (e.g., in a serum sample). In some embodiments, the method includes determining a level of LAG-3 and CXCL13 (e.g., in a serum sample). In some embodiments, the method includes determining a level of TIM-3, LAG-3 and CXCL13 (e.g., in a serum sample).

These levels provide information regarding the subject's severity and extent of infection. For example, as disclosed herein, osteomyelitis is associated with significantly elevated soluble LAG-3 serum titer.

These levels also provide information regarding the subject's likelihood of experiencing an adverse outcome, e.g., mortality, e.g., within a specific time period, e.g., 30 days, 60 days, 90 days, 6 months, one year, two years, three years, or five years. For example, as disclosed herein, elevated levels of soluble TIM-3 in the serum of subjects prior to receiving surgery or an implant are significantly associated with adverse and unfavorable outcomes in the subject post surgery and implant. These levels also provide information regarding the severity of disease in the subject. In some embodiments, the level of a biomarker may be determined once, e.g., at or before an event of suspected S. aureus infection (such as a TJR surgery). In some embodiments, the level of a biomarker is be determined 2, 4, 6, 8, 12, 18, and/or 24 hours, and/or 1-7 days after the infection event. Where more than one level is determined, the level of a biomarker may be calculated that quantifies whether and how much the levels of one or more biomarkers (e.g., TIM-3, LAG-3, PD-1, CTLA-4, IFNγ, IL-2, TNFα, IL-17) in the subject has increased or decreased.

Evaluating circulating levels of one or more biomarkers in a subject typically includes obtaining a tissue or biological sample, e.g., serum, plasma, or blood, from the subject. A tissue or biological sample from the subject can be obtained by any known means, including needle stick, needle biopsy, swab, and the like. In one aspect, the biological sample is a blood sample, preferably a blood plasma or serum sample, which is obtained, for example, by venipuncture. In a further aspect, the biological sample is a blood serum sample. Biological samples may be or may have been stored or banked under suitable tissue storage conditions.

Levels of one or more biomarkers in the sample can routinely be determined by measuring levels of a polypeptide in the sample using methods known in the art and/or described herein, e.g., immunoassays such as ELISA. The antibodies measured can include antigen-binding fragments thereof, degradation products thereof, and/or enzymatic cleavage products thereof. Alternatively, levels of mRNAs encoding corresponding antibody chains can routinely be measured using methods known in the art and/or described herein, e.g., by quantitative PCR or Northern blotting analysis.

In some embodiments, the method includes determining the presence, concentration or amount of one or more biomarkers. The presence, concentration, or amount, or a ratio thereof, of one or more biomarkers in a biological sample may be routinely determined using any suitable assay as is known in the art. Examples include, but are not limited to, immunoassays, such as sandwich immunoassay (e.g., monoclonal-polyclonal sandwich immunoassays, including radioisotope detection (radioimmunoassay (RIA)) and enzyme detection (enzyme immunoassay (EIA) or ELISA (e.g., Quantikine ELISA assays, R&D Systems, Minneapolis, Minn.)), competitive inhibition immunoassay (e.g., forward and reverse), fluorescence polarization immunoassay (FPIA), enzyme multiplied immunoassay technique (EMIT), bioluminescence resonance energy transfer (BRET), and homogeneous chemiluminescent assay, etc. In a SELDI-based immunoassay, a capture reagent that specifically binds an antibody (or a fragment thereof) off interest is attached to the surface of a mass spectrometry probe, such as a pre-activated protein chip array. Other suitable methods include, for example, mass spectrometry and immunohistochemistry (e.g., with sections from tissue biopsies) using antibodies (such as monoclonal, polyclonal, chimeric, humanized, human antibodies, etc.) or fragments thereof against angiopoietin. Other methods of detection include those described in, for example, U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety.

Immobilized capture proteins (e.g., antibodies specific for immune checkpoint molecules) or fragments thereof may be incorporated into the immunoassay. The capture proteins may be immobilized onto various supports, such as magnetic or chromatographic matrix particles, the surface of an assay plate (such as microtiter wells), pieces of solid substrate material, and the like. An assay strip can be prepared by coating the capture proteins in an array on a solid support. This strip can then be dipped into the biological test sample and then processed quickly through washes and detection steps to generate a measurable signal, such as a colored spot. Any solid support known in the art can be used, including but not limited to, solid supports made out of polymeric materials in the forms of wells, tubes, or beads. The capture proteins or antibodies can be bound to the solid support by adsorption, by covalent bonding using a chemical coupling agent or by other means known in the art, provided that such binding does not interfere with the binding ability of the capture proteins. Moreover, if necessary, the solid support can be derivatized to allow reactivity with various functional groups on the proteins. Such derivatization requires using certain coupling agents such as, but not limited to, maleic anhydride, N-hydroxysuccinimide, and 1-ethyl-3-(3-dimethyl aminopropyl) carbodiimide.

Additional testing may be performed to determine the subject's actual condition. More aggressive treatment may be administered either before or after additional testing. For example, in the case of suspected sepsis, the subject may be sent for more extensive imaging examinations.

With respect to a predetermined value or level as employed for monitoring disease progression and/or treatment, the amounts/concentration of one or more biomarkers or a ratio thereof may be “unchanged,” “favorable” (or “favorably altered”), or “unfavorable” (or “unfavorably altered”). “Elevated” or “increased” refers to an amount or a concentration or a ratio in a test sample that is higher than a typical or normal level or range (e.g., predetermined value/level), or is higher than another reference level or range (e.g., earlier or baseline sample). The term “lowered” or “reduced” refers to an amount or a concentration or ratio in a test sample that is lower than a typical or normal level or range (e.g., predetermined value/level), or is lower than another reference level or range (e.g., earlier or baseline sample). The term “altered” refers to an amount, a concentration, or a ratio in a sample that is altered (increased or decreased) over a typical or normal level or range (e.g., predetermined value/level), or over another reference value/level or range (e.g., earlier or baseline sample).

The typical or normal value, level, or range for a ratio is defined in accordance with standard practice. A so-called altered level or alteration can be considered to have occurred when there is any net change compared to the typical or normal level or range, or reference level or range that cannot be explained by experimental error or sample variation. Thus, the level measured in a particular sample will be compared with the level or range of levels determined in similar samples from a so-called normal subject. In this context, a “normal subject” is an individual with no detectable disease or disorder, and a “normal” (sometimes termed “control”) patient or population is/are one(s) that exhibit(s) no detectable disease or disorder, respectively, for example. The level of an analyte is said to be “elevated” when the analyte is normally undetectable (e.g., the normal level is zero, or within a range of from about 25 to about 75 percentiles of normal populations), but is detected in a test sample, as well as when the analyte is present in the test sample at a higher than normal level. Thus, inter alia, the disclosure provides a method of screening for a subject having or at risk of having S. aureus infections (e.g., sepsis).

Information obtained from the methods described above is useful in prognostication, identifying progression, and clinical management of diseases and other deleterious conditions affecting an individual subject's health status. In some embodiments, the information is useful in prognostication, identifying progression, and management of total joint replacement surgery and post-surgery care. The information more specifically assists the clinician in designing suitable treatment regimens to treat or prevent conditions such as S. aureus osteomyelitis and related sepsis or septic death of an afflicted subject.

The term “prognosis,” as used herein, refers to the prediction of the probable course and outcome of a clinical condition or disease, such as the likelihood of S. aureus osteomyelitis and related sepsis or septic death or its progression, including recurrence and drug resistance. A prognosis is usually made by evaluating factors or symptoms of a disease that are indicative of a favorable or unfavorable course or outcome of the disease.

The term “prediction” is used herein to refer to the likelihood that a patient will respond either favorably or unfavorably to a treatment, and also the extent of those responses, or that a patient will survive, following a treatment (such as a TJR surgery) for a certain period of time. The predictive methods of the present invention can be used clinically to make treatment decisions by choosing the most appropriate treatment modalities for any particular patient. The predictive methods described herein are valuable tools in predicting if a patient is likely to respond favorably to a treatment regimen (such as surgical intervention), or whether long-term survival of the patient, following surgery and/or termination of other treatment modalities is likely.

The phrase “determining the prognosis,” as used herein, refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “favorable prognosis” and “positive prognosis,” or “unfavorable prognosis” and “negative prognosis” as used herein are relative terms for the prediction of the probable course and/or likely outcome of a condition or a disease. A favorable or positive prognosis predicts a better outcome for a condition than an unfavorable or negative prognosis. In a general sense, a “favorable prognosis” is an outcome that is relatively better than many other possible prognoses that could be associated with a particular condition, whereas an unfavorable prognosis predicts an outcome that is relatively worse than many other possible prognoses that could be associated with a particular condition. Typical examples of a favorable or positive prognosis include a better-than-average cure rate, a lower propensity for osteomyelitis and related sepsis or septic death, and the like. For example, a positive prognosis is one where a patient has a 50% probability of being cured of osteomyelitis and related sepsis after treatment, while the average patient with the same condition has only a 25% probability of being cured.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

The term “biomarker,” as used herein, refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features, and/or may serve as an indicator of a particular cell type or state (e.g., epithelial, mesenchymal etc.) and/or response to therapy. Biomarkers include, but are not limited to, polynucleotides (e.g., DNA and/or RNA), polynucleotide copy number alterations (e.g., DNA copy numbers), polypeptides, polypeptide and polynucleotide modifications (e.g., posttranslational modifications), carbohydrates, and/or glycolipid-based molecular markers. A biomarker may be present in a sample obtained from a subject before the onset of a physiological or pathophysiological state (e.g., primary cancer, metastatic cancer, etc.), including a symptom thereof (e.g., response to therapy). Thus, the presence of the biomarker in a sample obtained from the subject can be indicative of an increased risk that the subject will develop the physiological or pathophysiological state or symptom thereof. Alternatively and/or additionally, the biomarker may be normally expressed in an individual, but its expression may change (i.e., it is increased (upregulated; over-expressed) or decreased (downregulated; under-expressed) before the onset of a physiological or pathophysiological state, including a symptom thereof. Thus, a change in the level of the biomarker may be indicative of an increased risk that the subject will develop the physiological or pathophysiological state or symptom thereof. Alternatively, or in addition, a change in the level of a biomarker may reflect a change in a particular physiological or pathophysiological state, or symptom thereof, in a subject, thereby allowing the nature (e.g., severity) of the physiological or pathophysiological state, or symptom thereof, to be tracked over a period of time.

In some embodiments, a level of a biomarker includes the concentration of the biomarker, the level of expression of the biomarker, or the activity of the biomarker.

The terms “level of expression” or “expression level” in general are used interchangeably and generally refer to the amount of a biomarker in a sample. “Expression” generally refers to the process by which information (e.g., gene-encoded and/or epigenetic) is converted into the structures present and operating in the cell. Therefore, as used herein, “expression” refers to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis.

As used herein, the terms “decrease,” “reduce,” and “inhibit” all generally refer to a decrease by a statistically significant amount. However, for avoidance of doubt, the term “reduced,” “decrease,” “reduce,” or “inhibit” means a decrease by at least 5% (e.g., 5%, 10%, 15%, 20%, 25%, 30%, 35%, 40%, 45%, 50%, 55%, 60%, 65%, 70%, 75%, 80%, 85%, 90%, 95%, 100%) as compared to a reference level, for example, a decrease by at least about 10%, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease of 10-100% as compared to a reference level. In some embodiments, these terms refer to a decrease of 10-20%, 10-30%, 10-40%, 10-50%, 10-60%, 10-70%, 10-80%, 10-90%, 10-100%, 10-110%, 10-120%, 10-130%, 10-140%, 10-150%, 10-160%, 10-170%, 10-180%, 10-190%, 10-200%, 10-210%, 10-220%, 10-230%, 10-240%, 10-250%, 10-260%, 10-270%, 10-280%, 10-290%, or 10-300%, as compared to a reference level. In some embodiments, these terms refer to a decrease of 10-300%, 20-300%, 30-300%, 40-300%, 50-300%, 60-300%, 70-300%, 80-300%, 90-300%, 100-300%, 110-300%, 120-300%, 130-300%, 140-300%, 150-300%, 160-300%, 170-300%, 180-300%, 190-300%, 200-300%, 210-300%, 220-300%, 230-300%, 240-300%, 250-300%, 260-300%, 270-300%, 280-300%, or 290-300%, as compared to a reference level. In some embodiments, these terms refer to a decrease of 10-20%, 20-30%, 30-40%, 40-50%, 50-60%, 60-70%, 70-80%, 80-90%, 90-100%, 100-110%, 110-120%, 120-130%, 130-140%, 140-150%, 150-160%, 160-170%, 170-180%, 180-190%, 190-200%, 200-210%, 210-220%, 220-230%, 230-240%, 240-250%, 250-260%, 260-270%, 270-280%, 280-290%, or 290-300%, as compared to a reference level.

An “antigen” refers to a substance that elicits an immunological reaction or binds to the products of that reaction. The term “epitope” refers to the region of an antigen to which an antibody or T cell binds.

A “patient” or “subject” includes an animal, such as a human, cow, horse, sheep, lamb, pig, chicken, turkey, quail, cat, dog, mouse, rat, rabbit, or guinea pig. The animal can be a mammal, such as a non-primate and a primate (e.g., monkey and human). In one embodiment, a patient is a human, such as a human infant, child, adolescent or adult. A “subject in need thereof” refers to a subject at risk of, or suffering from, a disease, disorder, or condition (e.g., hyperproliferative disorder like cancer, chronic infection) that is amenable to treatment or amelioration with a compound or a composition thereof provided herein. Subjects in need of administration of therapeutic agents as described herein include subjects suspected of having a cancer, subjects presenting with an existing cancer, subjects receiving a cancer vaccine, subjects suspected of being infected with an infectious agent, subjects presenting with an infection or infectious disease, or subjects receiving a vaccine against an infectious agent. A subject may be any organism capable of developing cancer or being infected, such as humans, pets, livestock, show animals, zoo specimens, or other animals. For example, a subject may be a human, a non-human primate, dog, cat, rabbit, horse, or the like. In some embodiments, a subject in need is a human. In particular embodiments, a subject in need has a disease, such as cancer or chronic infection, associated with immune resistance.

A “control subject” is a healthy subject, i.e., a subject having no clinical signs or symptoms of sepsis. Preferably a control subject is clinically evaluated for otherwise undetected signs or symptoms of sepsis, which evaluation includes routine laboratory testing. Preferably in healthy control subjects is the mean or average titer of soluble TIM-3 in at least 10, 50 or 100 subjects.

A “reference subject” or “reference population” defines the source of a reference standard. In one embodiment, the reference is a human subject or population of subjects with one or more clinical indicators of S. aureus infection, but who did not develop S. aureus osteomyelitis or sepsis. In another embodiment, the reference is a human subject or a population of subjects who had S. aureus infection, S. aureus osteomyelitis or sepsis, but survived without treatment. In another embodiment, the reference is a human subject or a population of subjects who had S. aureus infection, S. aureus osteomyelitis or sepsis, but survived with treatment. The above reference subjects or populations can be used to obtain the first predetermined reference mentioned above.

In another embodiment, the reference is a human subject or a population of subjects who had S. aureus infection, S. aureus osteomyelitis or sepsis, and did not survive without treatment. In still another embodiment, the reference is a human subject or a population of subjects who had S. aureus infection, S. aureus osteomyelitis or sepsis, and did not survive even with treatment. These reference subjects or populations can be used to obtain the second predetermined reference mentioned above.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can routinely be evaluated using a variety of methods known to the skilled practitioner, such as in human subjects during clinical trials, in animal model systems predictive of efficacy in humans, or by assaying the activity of the agent in in vitro assays.

As used herein, “administering” refers to the physical introduction of a composition comprising a therapeutic agent to a subject, using any of the various methods and delivery systems known to those skilled in the art. In some embodiments, routes of administration for therapeutic agents (e.g., antibodies) described herein include intravenous, intraperitoneal, intramuscular, subcutaneous, spinal or other parenteral routes of administration, for example, by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a therapeutic agent described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render them suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder or pathological condition; reducing or preventing the onset of a disease, symptom, disorder or condition; and generally counteracting a disease, symptom, disorder or pathological condition.

The term “disease” as used herein is intended to be generally synonymous and is used interchangeably with the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Prophylaxis refers to administration to a subject who does not have a disease to prevent the disease from occurring or minimize its effects if it does.

The terms “prevent,” “preventing,” “prevention,” “prophylactic treatment,” and the like refer to reducing the probability of developing a disorder or condition in a subject, who does not have, but is at risk of or susceptible to developing a disorder or condition. The term includes prevention of the spread of infection in a subject.

As used herein, the term “ameliorating” refers to relieving the symptoms of a disease, disorder or condition in a subject already exhibiting the symptoms of the disease, disorder and/or condition, i.e., causing regression of the disease, disorder and/or condition that has already affected the subject.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. Blood 117, 2423 (2011).

The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample,” as used herein, generally refer to a biological material being tested for and/or suspected of containing an analyte of interest, such as antibodies. The sample may be any tissue sample from the subject. The sample may comprise protein from the subject. Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or fluid collected by vaginal flushing. A tissue or cell type may be provided by removing a sample of cells from an animal, but it can also be accomplished by using previously isolated cells (e.g., isolated by another person, at another time, and/or for another purpose). Archival tissues, such as those having treatment or outcome history, may also be used. Protein purification may not be necessary.

In some embodiments, the sample is serum. In some embodiments, the sample contains bone marrow cells. In some embodiments, the sample contains blood cells (e.g., peripheral blood mononucleated cells (PBMCS), neutrophils, metamyelocytes, monocytes, or T cells)). In further embodiments, the sample contains T cells, e.g., Th1/Th17 cells.

Doses are often expressed in relation to body weight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

As used herein, “in vitro” refers to events that occur in an artificial environment, e.g., in a test tube or reaction vessel, in cell culture, etc., rather than within a multi-cellular organism.

As used herein, “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

As used herein, “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like do not necessarily refer to the same embodiment, but may unless the context dictates otherwise.

As used herein, the terms “and/or” or “/” means any one of the items, any combination of the items, or all of the items with which this term is associated.

As used herein, the term “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the invention and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1

S. aureus osteomyelitis is broadly considered to be incurable due to recalcitrant biofilms and colonization of the osteocyte-lacuno canalicular network (OLCN) of cortical bone, which cannot be eradicated with standards of care short of amputation (Masters, E. A. et al. Nat Rev Microbiol 20, 385-400 (2022)). However, it is also known that patients can resolve acute infections and live a full lifetime with asymptomatic S. aureus osteomyelitis (Masters, E. A. et al. Bone Res 7, 20 (2019)). Unfortunately, currently available diagnostics to guild this critical decision are very limited, which led the 2018 International Consensus Meeting on Musculoskeletal Infection to conclude that development of a functional definition for acute vs. chronic osteomyelitis is the greatest priority in this field (Schwarz, E. M. et al. J Orthop Res 37, 997-1006 (2019)).

To this end, preclinical natural history studies evaluated transitions in host immunity and found that initial robust pro-inflammatory responses in the acute phase of infection transition from Th1 and Th17 to suppressive Treg adaptive immune responses over time (Sokhi, U. K. et al. J Bone Miner Res (2021)). Since S. aureus is a human-specific pathogen, and immune responses against this pathogen vary between people and animal models, a novel model was recently developed, in which non-obese diabetic (NOD)-SCID IL2Rgamma (null) (NSG) mice were engrafted with human hematopoietic stem cells (huNSG), and the fate of the human immune cells in response to S. aureus implant-associated infection was studied over time (Muthukrishnan, G. et al. Front Immunol 12, 651515 (2021)).

As demonstrated in this example, improvements to the humanized mouse model were made to study the entire T cell repertoire during S. aureus bone infections. Humanized BLT (bone marrow, liver, thymus; BLT model) mice were generated by engrafting transgenic NSG mice encoding human stem cell factor (SCF), granulocyte-macrophage colony-stimulating factor (GM-CSF), and interleukin-3 (IL-3) (NSG-SGM3) with human hematopoietic stem cells (CD34+), autologous fetal liver, and thymus and subjected to S. aureus transtibial osteomyelitis. The following results were observed.

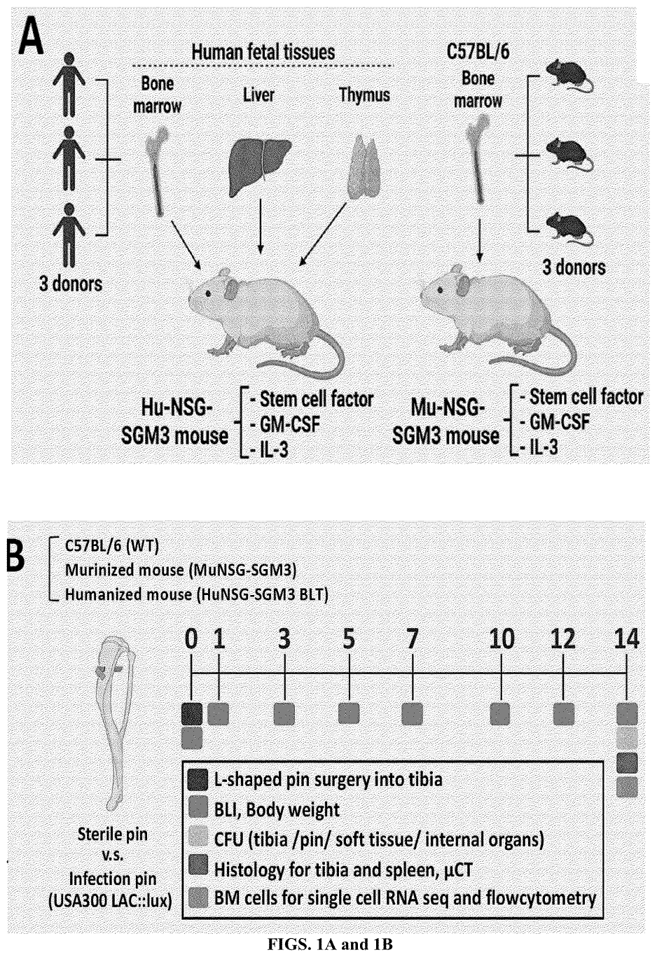

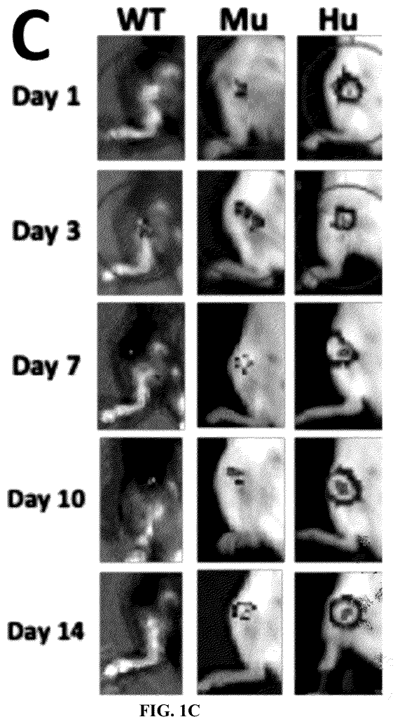

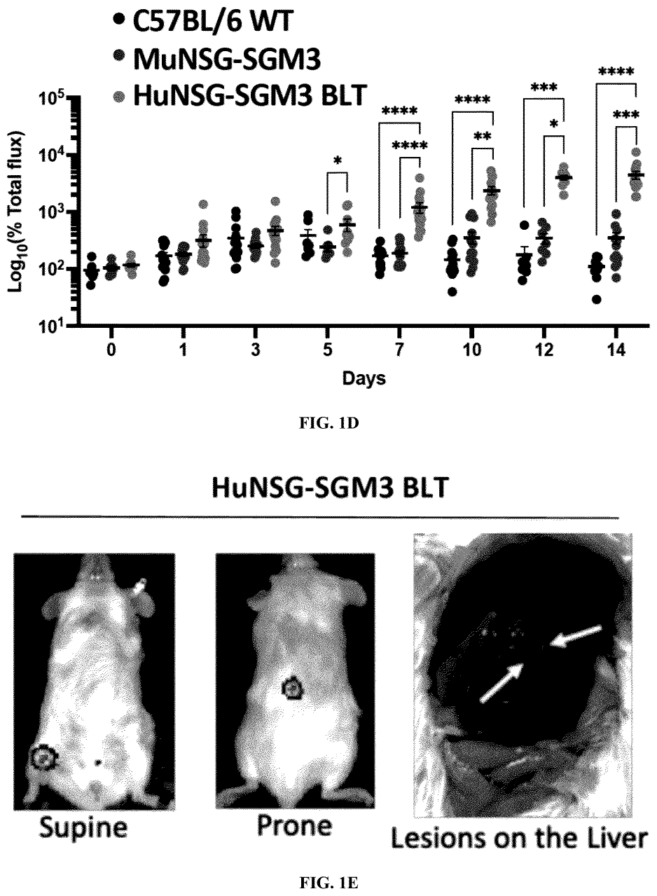

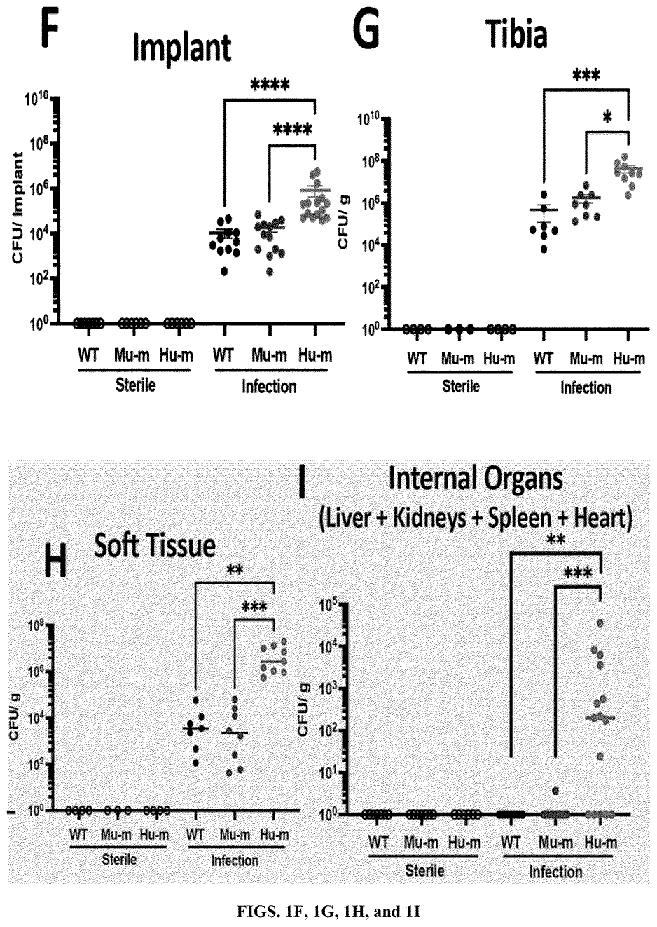

First, S. aureus infection was much more severe in huNSG-SGM3 BLT mice compared to murinized NSG-SGM3 and C57BL6 (WT) mice (FIG. 1). Moreover, huNSG-SGM3 BLT mice exhibited enhanced MRSA dissemination to distal organs suggesting that these mice were septic to S. aureus osteomyelitis.

To that end, humanized NSG-SGM3 BLT mice were generated by engrafting with CD34+ human hematopoietic cells, autologous human fetal liver, and thymus from three different human donors. Murinized NSG-SGM3 BLT mice were generated with CD34+ murine hematopoietic cells derived from three different C57BL/6 WT mice. 20-week-old humanized NSG-SGM3 BLT mice, murinized NSG-SGM3 and C57BL6 (WT) mice were subjected to transtibial implant-associated osteomyelitis using bioluminescent MRSA (USA300 LAC::lux). As shown in FIGS. 1C and 1D, longitudinal assessments of in vivo S. aureus growth via bioluminescent imaging revealed increased in vivo S. aureus growth in humanized NSG-SGM3 BLT mice. Moreover, as shown in FIG. 1E, MRSA dissemination from the site of infection to distal internal organs was observed. Also, on day 14 post-operation, the huNSG-SGM3 BLT mice and control animals were euthanized, and ex vivo CFU quantification was performed on the implants, tibia, soft tissues surrounding the tibia, and internal organs (heart, liver, kidneys, and spleen). As shown in FIGS. 1F-1I, CFU quantitation revealed that huNSG-SGM3 BLT mice exhibited exacerbated susceptibility and increased sepsis due to S. aureus osteomyelitis compared to control murinized NSG-SGM3 and C57BL6 (WT) animals (n=25 (3 human donors), ANOVA, *p<0.05, ** p<0.01, *** p<0.001, *** p<0.0001).

Post euthanasia, MRSA-infected and sterile implant-inserted tibiae from a subset of huNSG-SGM3 BLT, NSG-SGM3, and C57BL/6 animals were demineralized, sectioned, and processed for histopathological analyses. Brown & Brenn staining for identifying bacteria revealed numerous SACs formation and increased SAC area over the tibial area in humanized BLT mice, compared to control animals (n=4-5, ANOVA, *p<0.05). See FIGS. 1J-1K.

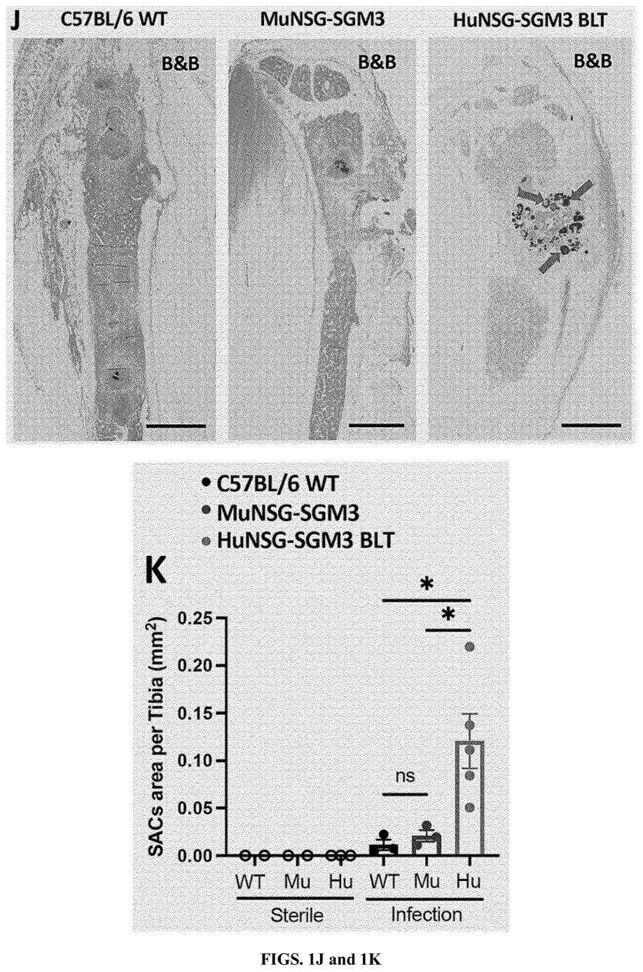

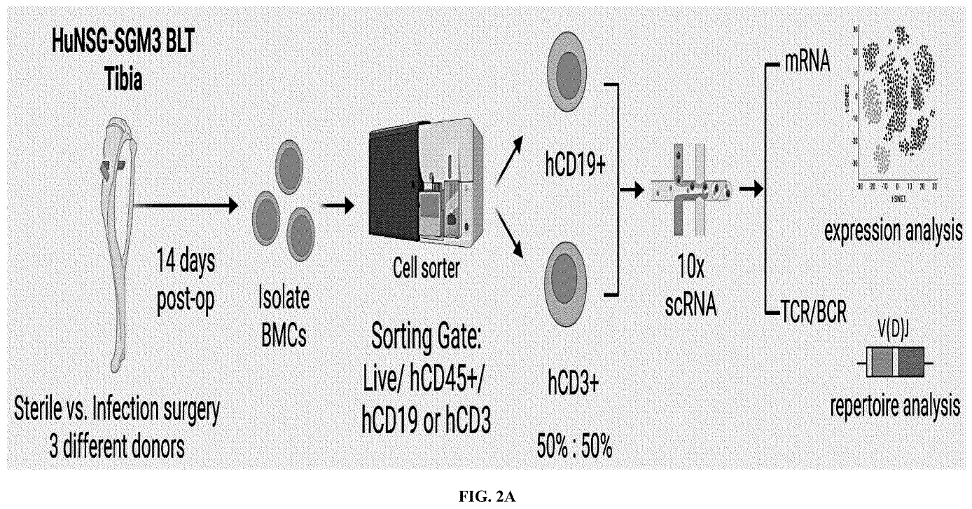

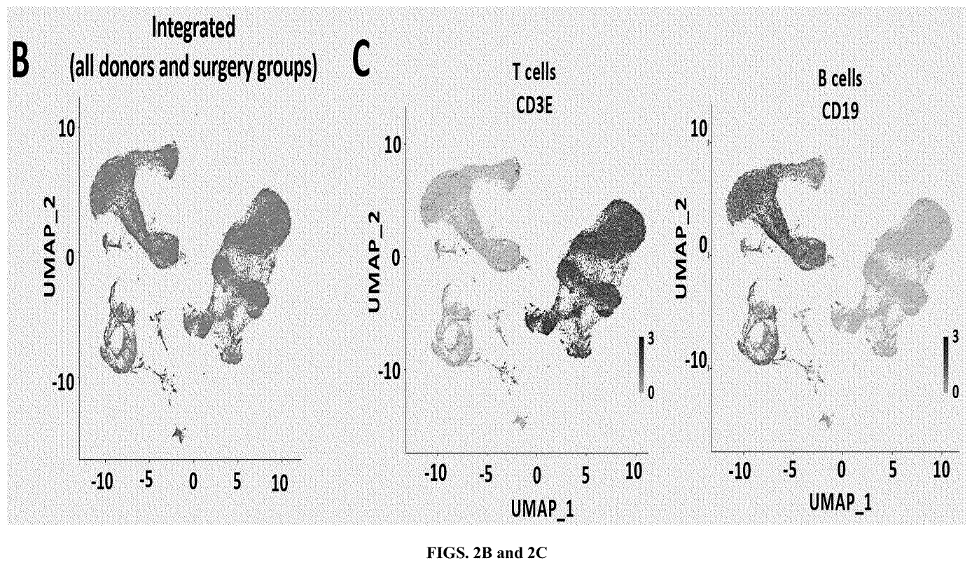

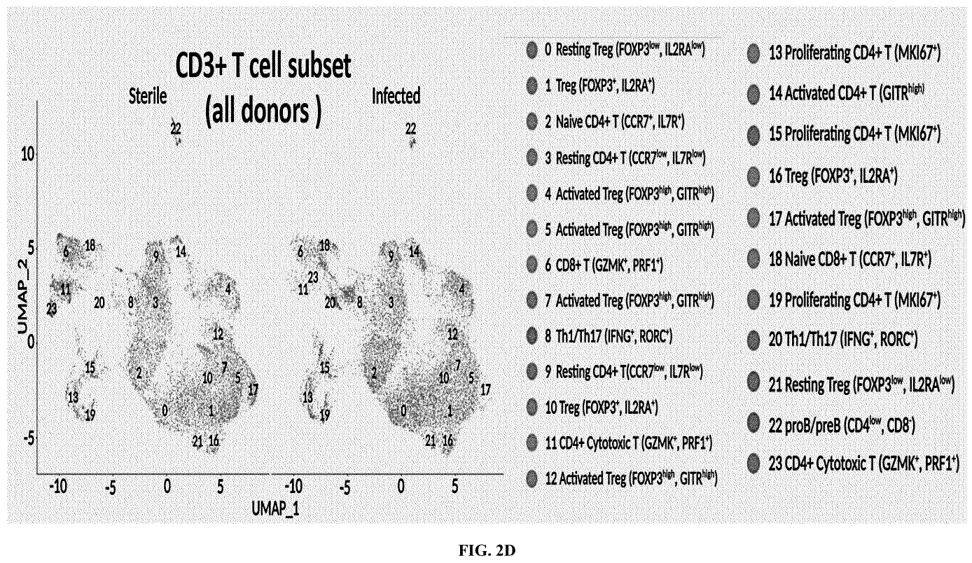

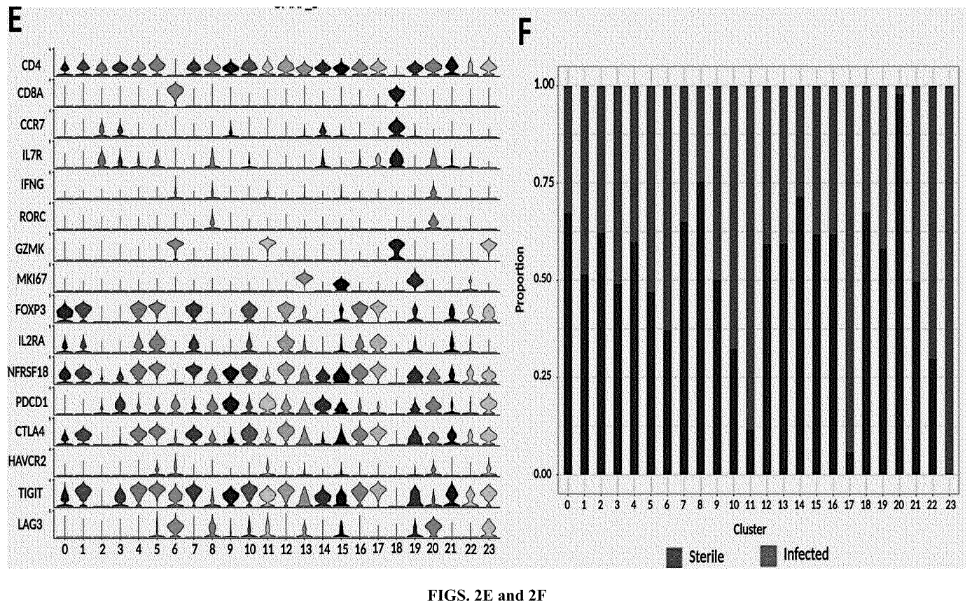

Second, single-cell RNAseq analyses of the human T (CD3+) cell population in the huNSG-SGM3 BLT mice bone marrow revealed remarkable heterogeneity in gene expression (FIGS. 2A-B) and T cell population numbers (FIG. 2C) between sterile and infection surgery groups.

More specifically, bone marrow cells were isolated from tibiae of humanized NSG-SGM3 BLT mice that underwent surgery with or without bioluminescent MRSA-contaminated transtibial implant on day 14 post-operation. The isolated bone marrow cells were FACS sorted into human CD45+CD19+ B cells and CD45+CD3+ T cells. Equal proportions of the B and T cells were subjected to sc-RNAseq and sc-TCR/BCR repertoire analyses. As shown in FIG. 2B, UMAP plot of single-cell gene expression of T and B cells in all ~30,000 BM cells from humanized NSG-SGM3 BLT mice tibiae were generated. Shown in FIG. 2C are feature plots of pan T cell marker, CD3E and B cell marker, CD19 in all integrated BM cells. As shown in FIGS. 2D and 2E, UMAP and DEG clustering analyses of hCD45+ CD3+ T cells identified 24 T cell clusters. Bar plot displaying the proportion of cell counts in each cluster between sterile sham surgery and infected implant groups was generated and shown in FIG. 2F. It was found that the number of Th1/Th17 cells (Cluster 8,20) was prominently increased in the infected animals compared to sham surgery (sterile) animals.

### Example 2

In this example, assays were carried out to examine immune checkpoint protein levels in the mice. It was found that immune checkpoint proteins were elevated in the CD4+Th1/Th17 cells in S. aureus-infected humanized BLT mice tibia.

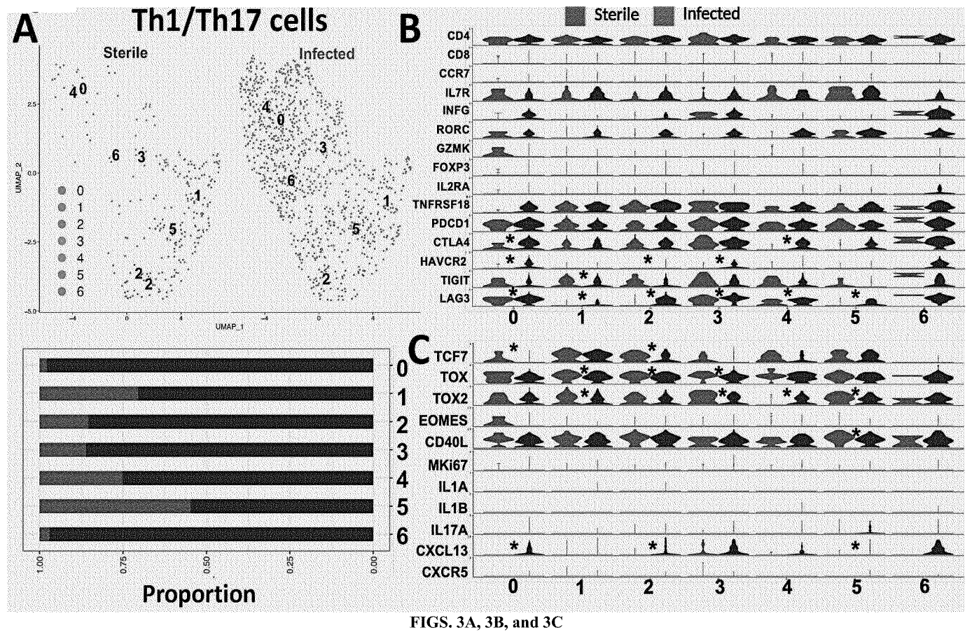

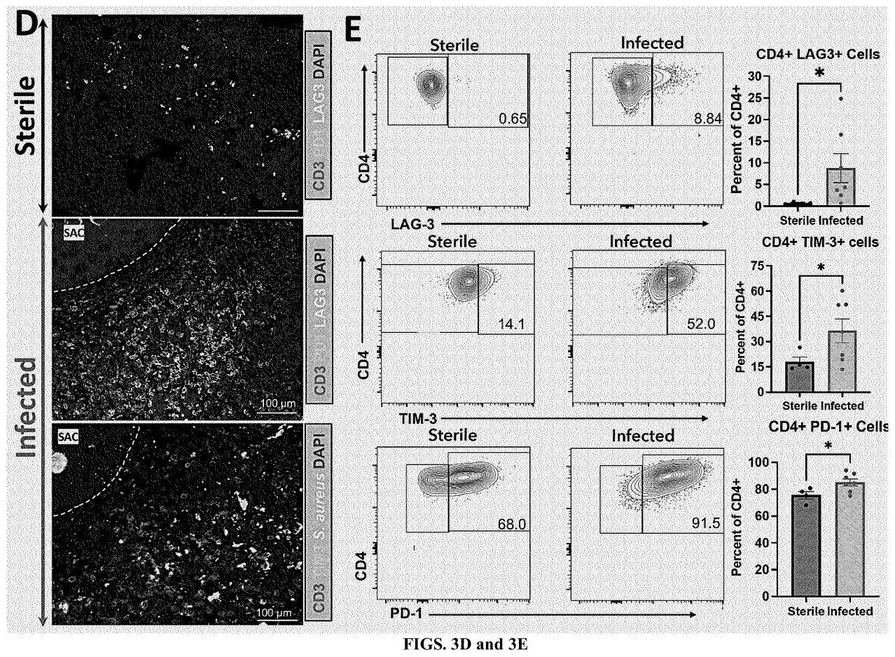

Briefly, Th1/Th17 cells (clusters 8 and 20 from FIG. 2D) were subjected to UMAP and differential gene expression analyses (DEG) sub-clustering analyses. The analyses revealed 7 clusters as shown in FIG. 3A. Bar plot analyses demonstrated that these cells were of the Th1/Th17 phenotype. See FIG. 3B. Several Th1/Th17 clusters showed significantly increased expression of immune checkpoint molecules LAG3, TIM-3 (HAVCR2), and, to a lesser extent, CTLA-4 and other immunosuppressive genes like TIGIT.

Shown in FIG. 3C were DEG analyses of transcriptional factors (TCF7, TOX1-2, EOMES, NR4A1), cytokines & chemokines (IL-1, IL-17, CXCL13, CXCR5) associated with functional T cell exhaustion, chronic antigenic stimulation (CD40L) and proliferation (MKi67). The lower expression of TCF7, MKi67, IL-1, and IL-17 genes and higher expression of CXCL13 and TOX 2 indicate transcriptional reprogramming of these cells to a terminally functionally exhausted state (*p<0.05).

Immunofluorescent analyses of tibia sections from sham surgery control and humanized BLT mice showed a large accumulation of LAG3+, TIM-3+, and/or PD-1+ T cells near the infection site in the MRSA-infected BLT mice. See FIGS. 3D and 3E. In contrast, human T cell accumulation was scant in uninfected BLT mice, and there was evidence of minimal exhaustion observed near the infection site.

A multichromatic spectral flow cytometry assay was developed, optimized, and performed on uninfected and MRSA-infected BLT mice's tibial bone marrow cells (BMs). The results are shown in FIG. 3E. Live human CD45+/CD3+/T cells and their subpopulations (CD4+, CD8+, Tregs) were analyzed for immune checkpoint expression (LAG3, TIM-3, and PD-1) and proliferation (Ki67). It was found that the frequency of human CD3+CD4+ T cells expressing TIM-3, LAG3 & PD-1) in the MRSA-infected BLT mice bone marrow cells were significantly higher compared to controls (n=4-8 mice, *p<0.05, t-test).

Differentially expressed genes (DEG) analyses revealed that immunosuppressive checkpoint signaling protein LAG-3 was significantly elevated Th17 and proliferating Th17 cells in the MRSA-infected humanized mice (FIG. 3). LAG-3 is also a well-known T cell exhaustion protein along with PD-1, TIM-3, and CTLA-4. These results suggest that human Th17 cell exhaustion could be attributed to increased susceptibility to osteomyelitis in the chronic stages of the S. aureus infection.

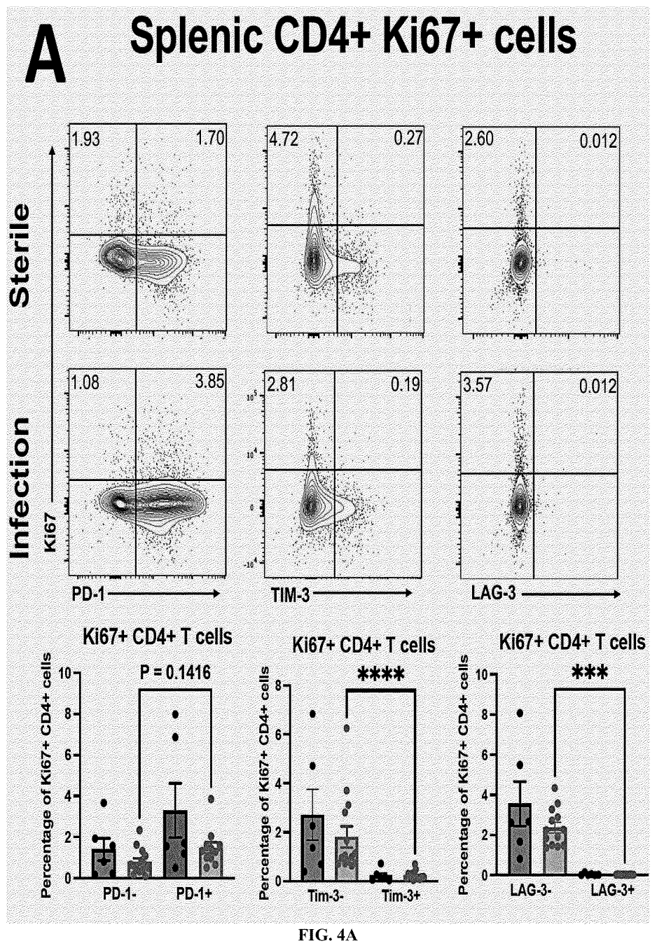

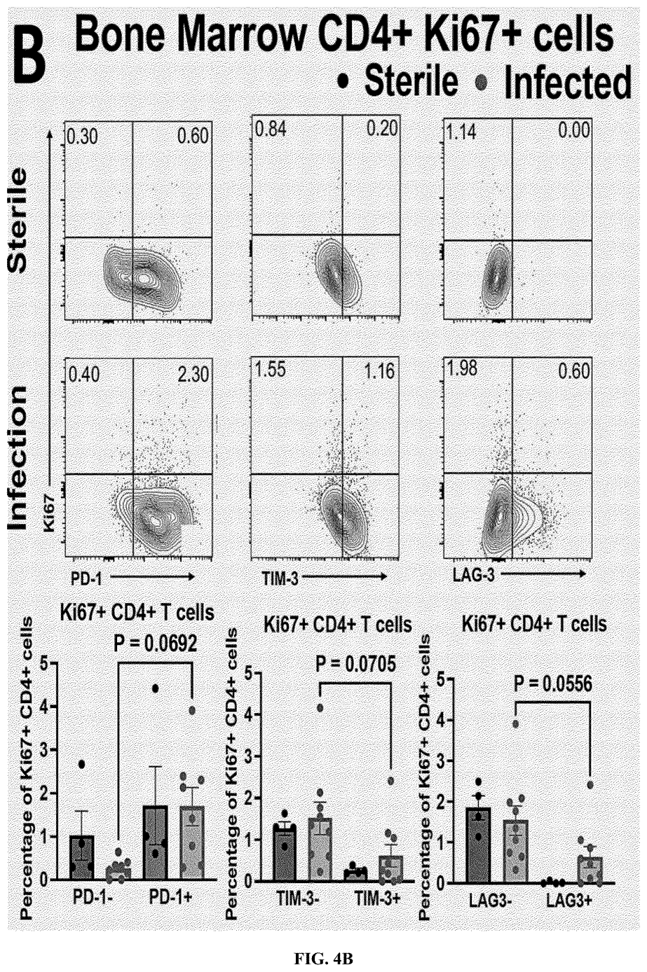

It was found that splenic and bone marrow CD4+ T cells expressing TIM-3 and LAG3 checkpoint proteins exhibit diminished proliferative capacity due to S. aureus infection. Briefly, multichromatic spectral flow cytometry on uninfected and MRSA-infected BLT mice (FIG. 4A) splenic cells and (FIG. 4B) tibial bone marrow cells was performed using protocols described herein. Subsequently, CD4+ T cell subpopulations expressing checkpoint molecules TIM-3, LAG3, and PD-1 were probed for their proliferative capacity using the cell surface marker Ki67. It was found that CD4+TIM-3+ and CD4+LAG3+ cells had a significantly lower frequency of proliferating Ki67+ cells in the spleen and trending lower amounts of proliferating Ki67+ cells in the bone marrow of infected BLT mice, suggesting functional exhaustion and dysfunction (n=4-9 mice, *p<0.05, ANOVA).

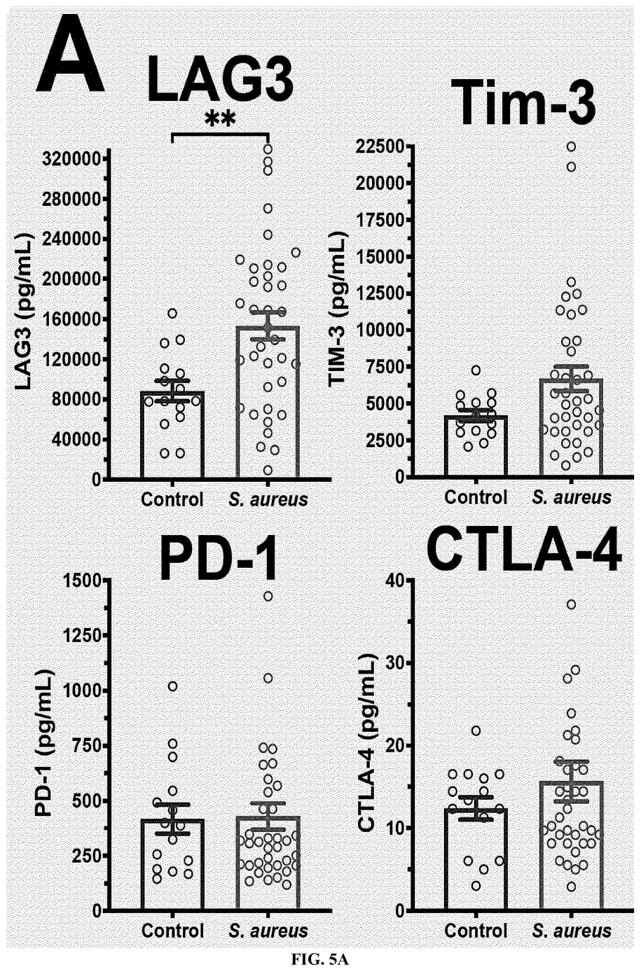

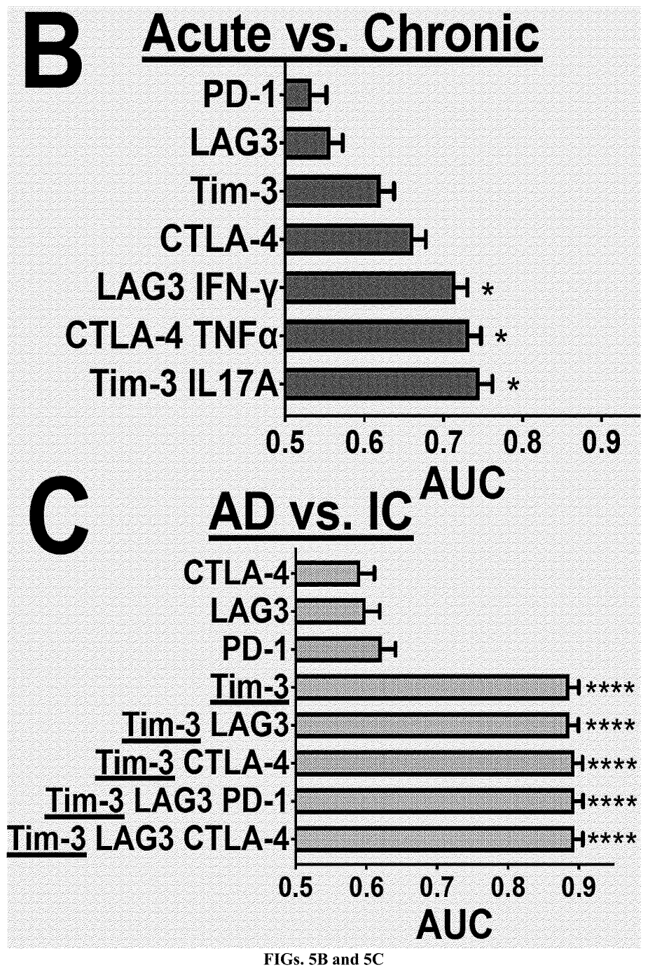

Most interestingly, serum LAG-3 levels were significantly elevated in patients with S. aureus osteomyelitis (FIG. 5). Moderate trending elevations in TIM-3 and CTLA-4 were also observed in the infected patients. Multivariate logistic regression analyses with risk characterized by odds ratios (OR calculated per 10-fold increase in serum protein levels) revealed that TIM-3 levels were significantly associated with adverse outcomes (OR=485.1, 95% CI 2.49-94511.09, p=0.02) such as arthrodesis, reinfection, amputation, and septic death. Serum TIM-3, in combination with LAG-3, PD-1, and CTLA-4, was highly predictive of adverse outcomes (AUC=0.89, p<0.00001) in osteomyelitis patients.

Briefly, serum samples were collected from individuals undergoing total hip/knee arthroplasty (n=15), orthopaedic patients with culture-confirmed S. aureus osteomyelitis with one-year (n=37, 12-Adverse Outcome (AD), 11-Infection Controlled (IC), 14-inconclusive). Immune checkpoint proteins LAG3, TIM-3, CTLA-4, PD-1 and cytokines (IFN-γ, IL-2, TNFα, IL-17A, IL-17F) were assessed by multiplex Luminex assay. The results are shown in FIG. 5, where data presented as the mean +/−SEM in each experimental group. The individual protein levels were utilized to perform receiver operating characteristic (ROC) curve analysis either singly or in combination to generate the area under the curve (AUC) for (FIG. 5B) differentiating acute vs. chronic S. aureus infections and (FIG. 5C) prognostic prediction of outcome. Interestingly, the no-correlation was observed between levels of immune checkpoint proteins and clinical time-based, anecdotal classification of acute vs. chronic classification. On the other hand, immune checkpoint proteins, especially Tim-3, were highly predictive of adverse in these patients (*p<0.05, ** p<0.01, **** p<0.00001).

The results indicate that the blockade of T cell exhaustion or suppression of immune checkpoint proteins can be used to treat patients with bone infections. For example, (1) anti-LAG-3 monoclonal antibody (40 mg/kg) or LAG-3 chemical inhibitor, and (2) anti-TIM-3 mAb (40 mg/kg) as monotherapy or combination with other immune checkpoint blockade therapies such as anti-PD-1, and anti-CTLA-4 can be useful in combating debilitating and incurable bone infections in patients. These immunotherapies can be adjuvant therapies prescribed in conjunction with clinical standard-of-care antibiotic treatments.

### Example 3

In this example, assays were carried out to examine the efficacy of immune checkpoint blockade drug OPDUALAG™, a cocktail of anti-PD-1 (nivolumab) and anti-LAG-3 (relatlimab) mAb, in reducing S. aureus burden in humanized BLT mice.

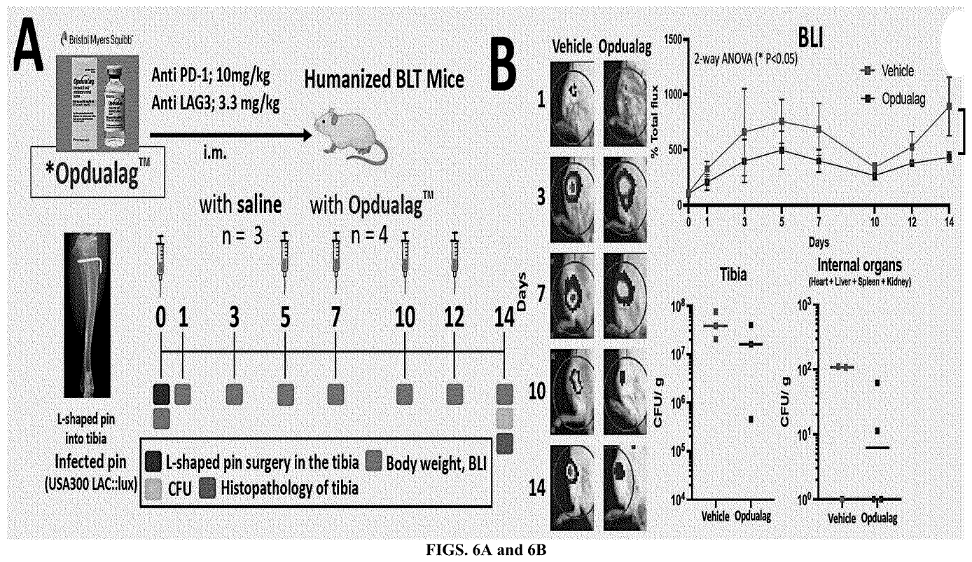

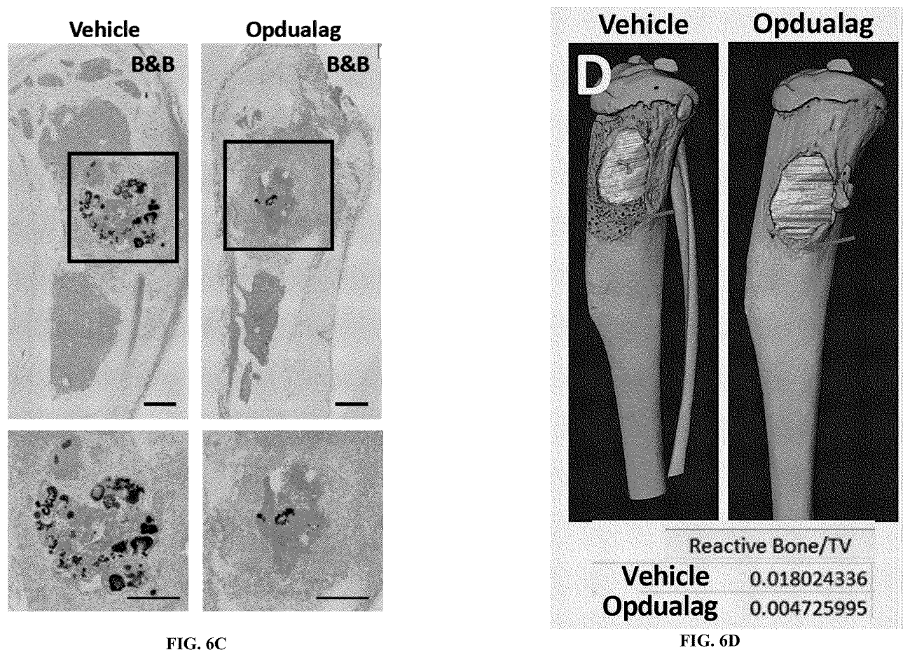

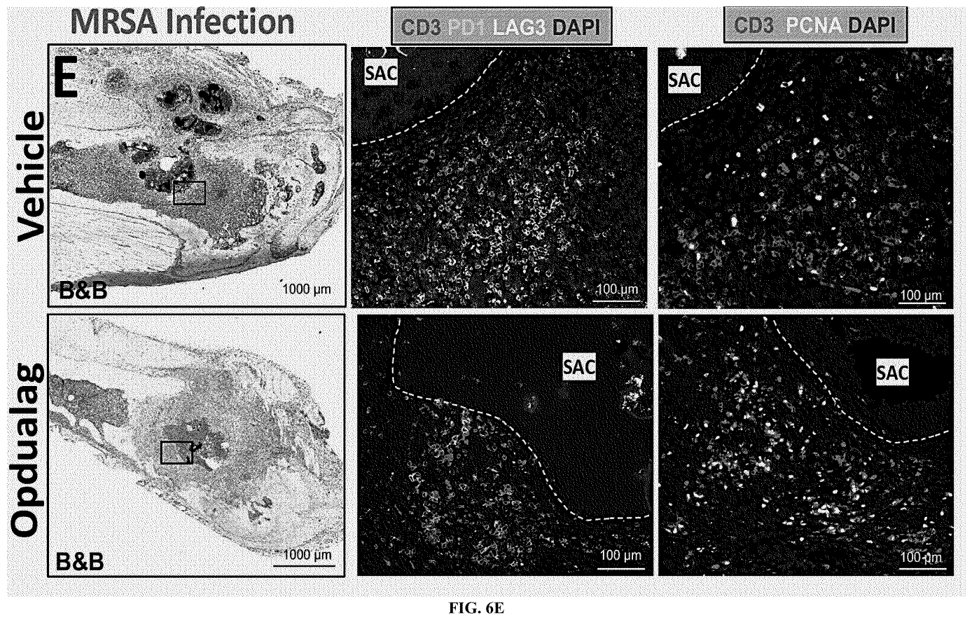

Briefly, 20-week-old humanized NSG-SGM3 BLT mice were subjected to transtibial implant-associated osteomyelitis using bioluminescent MRSA (USA300 LAC::lux). The mice were then treated with OPDUALAG™ or a saline placebo control. Post-infection, longitudinal body weight as a measure of morbidity and longitudinal bioluminescent imaging was performed on days 0, 1, 3, 5, 7, 10, and 14 to assess for the planktonic growth of MRSA. See FIG. 6A. Post-infection, the mice were sacrificed on day 14, and infection severity as a measure of (1) SAC formation (via histology) and (2) ex vivo CFU quantitation of infected tibiae, blood, liver, kidney, heart, and spleen was assessed between OPDUALAG™ or saline placebo control treated animals. The results were shown in FIGS. 6B and 6C.

As shown in FIG. 6B, longitudinal BLI revealed significantly decreased in vivo S. aureus growth in humanized BLT mice treated with OPDUALAG™ (n=3-4, ANOVA, *p<0.05). At day 14 post-surgery, terminal ex vivo CFU on the tibia and internal organs (heart, liver, kidneys, and spleen) revealed that OPDUALAG™-treated animals exhibited trending lower disease severity. As shown in FIG. 6C, histopathology revealed remarkably reduced SACs formation in the OPDUALAG™-treated animals compared to controls.

### Example 4

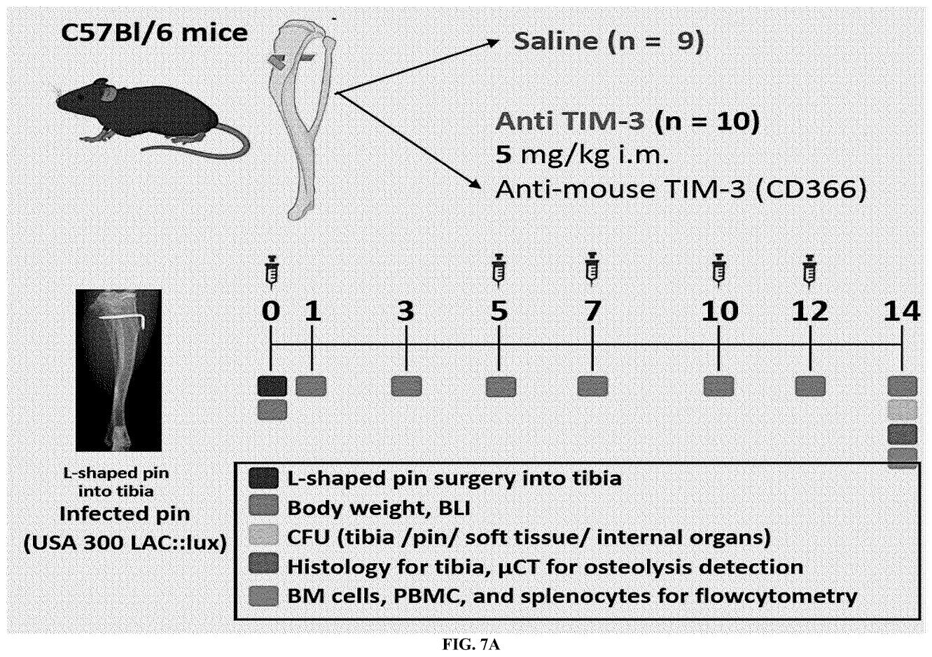

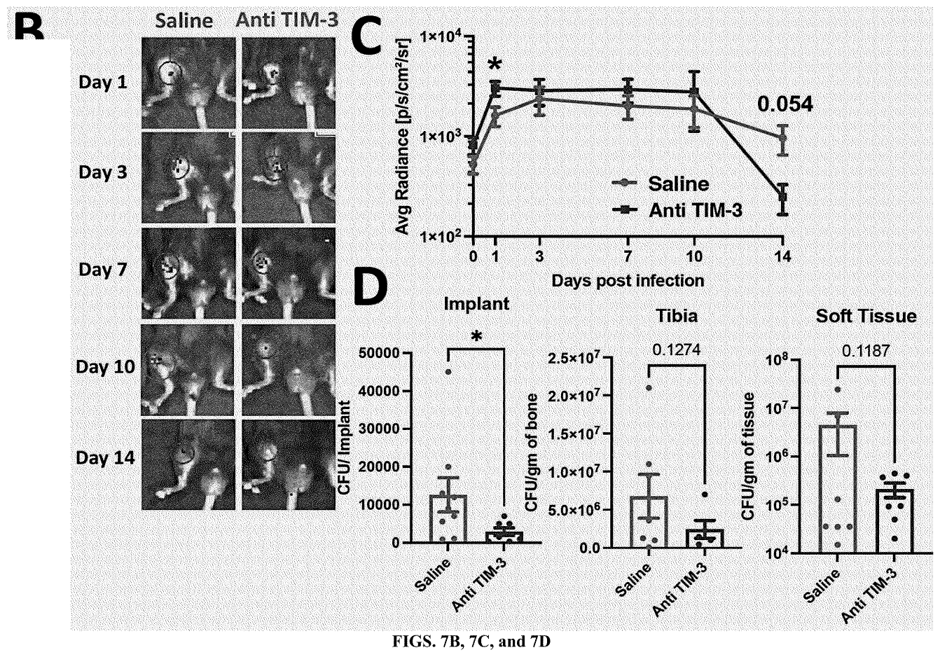

Similar assays were carried out to examine the efficacy trend of immune checkpoint blockade drug Sabatolimab (anti-TIM-3) in reducing S. aureus burden due to implant-associated osteomyelitis. The results were shown in FIGS. 7A, 7B, 7C, and 7D.

Briefly, 8-week-old C57BL/6 mice were subjected to transtibial implant-associated osteomyelitis using bioluminescent MRSA (USA300 LAC::lux). The mise were then treated with sabatolimab or a saline placebo control. The concentration of anti-TIM-3 mAb used in the study was also outlined in FIG. 7A. As shown in FIGS. 7B and 7C, longitudinal BLI revealed significantly decreased in vivo S. aureus growth in humanized BLT mice treated with sabatolimab (n=9-10, ANOVA, *p<0.05). In addition, at day 14 post-surgery, terminal ex vivo CFU on the tibia and internal organs (heart, liver, kidneys, and spleen) revealed that sabatolimab-treated animals exhibited lower disease severity (n=9−10, t-test, *p<0.05).

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method for reducing the number of cells of a pathogenic bacterium or a pathogenic fungus in a subject in need thereof, comprising administering to the subject an effective amount of an inhibitor of an inhibitory immune checkpoint molecule.

2. The method of claim 1, wherein the administered inhibitor inhibits an inhibitory immune checkpoint molecule selected from the group consisting of LAG-3, TIM-3, CTLA-4, PD-1, and PD-L1.

3. The method of claim 1, wherein the administered inhibitor comprises an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof.

4. The method of claim 1, wherein the administered inhibitor comprises an anti-LAG-3 antibody, an anti-TIM-3 antibody, an anti-CTLA-4 antibody, an anti-PD-1 antibody, an anti-PD-L1 antibody, an antigen-binding fragment thereof, or a combination thereof.

5. The method of claim 1, wherein the administered inhibitor comprises (i) an anti-LAG-3 antibody or an antigen-binding fragment thereof, (ii) a combination of an anti-PD-1 antibody or an antigen-binding fragment thereof and an anti-LAG-3 antibody or an antigen-binding fragment thereof, or (iii) a bispecific antibody that binds PD-1 and LAG-3.

6. (canceled)

7. A method of diagnosis or prognosis of a disease or disorder caused by a pathogenic bacterium or a pathogenic fungus in a subject in need thereof, comprising:
determining a level of each of a set of biomarkers in a sample from the subject, wherein the set of biomarkers comprises an inhibitory immune checkpoint molecule or a cytokine;
determining a change in the level of each of the set of biomarkers as compared to a reference level for each of the set of biomarkers; and
assessing the presence of the disease or disorder or a status of the disease or disorder based on the change in the level of each of the set of biomarkers as compared to the reference level for each of the set of biomarkers.

8. The method of claim 7, wherein the set of biomarkers comprises one or more of
(a) LAG-3, PD-1, CTLA-4, TIM-3, IFNγ, IL-2, TNFα, and IL-17; or
(b) LAG-3, TIM-3, and CXCL13.

9. The method of claim 7, wherein the set of biomarkers comprises:
(a) TIM-3; (b) TIM-3 and LAG-3; (c) TIM-3 and CTLA-4; (d) TIM-3 and PD-1; (e) TIM-3 and IL-17; (f) TIM-3, LAG-3, and PD-1; (g) TIM-3, LAG-3, and CTLA-4; (h) TIM-3, CTLA-4, and PD-1;
(i) TIM-3, LAG-3, CTLA-4, and PD-1; (j) TIM-3, LAG-3, and IL-17; (k) TIM-3, CTLA-4, and IL-17; (1) TIM-3, PD-1, and IL-17; (m) TIM-3, LAG-3, PD-1, and IL-17; (n) TIM-3, LAG-3, CTLA-4, and IL-17; (0) TIM-3, CTLA-4, PD-1, and IL-17; or (p) TIM-3, LAG-3, CTLA-4, PD-1, and IL-17.

10. (canceled)

11. The method of claim 7, wherein the sample is a bone marrow or PBMC sample or a serum sample.

12. (canceled)

13. The method of claim 7, wherein the change in the level of a set of biomarkers is an increase of an expression level of each of the biomarkers in the set.

14. A method of preventing, treating or ameliorating a disease or disorder caused by a pathogenic bacterium or a pathogenic fungus in a subject in need thereof, comprising administering to the subject a therapeutically effective amount of an inhibitor of an inhibitory immune checkpoint molecule.

15. (canceled)

16. The method of claim 7, wherein the disease or disorder comprises an infection.

17. The method of claim 16, wherein the infection is bacteremia, a bone infection, bone loss, osteomyelitis, biofilm formation, antimicrobial resistant infection, or sepsis or the infection is a prosthetic joint infection, a fracture related infection, a diabetic foot infection, a hematogenous osteomyelitis, or a spine infection.

18. (canceled)

19. The method of claim 14, wherein the administered inhibitor inhibits an inhibitory immune checkpoint molecule selected from the group consisting of LAG-3, TIM-3, CTLA-4, PD-1, and PD-L1.

20. The method of claim 14, wherein the administered inhibitor comprises an antibody or antigen-binding fragment thereof, a small molecule, a protein, a polypeptide, a peptide, a peptide mimetic, a nucleic acid, an antisense molecule, a ribozyme, a RNAi molecule, a lipid, a lipopeptide, a carbohydrate, or a combination thereof.

21-23. (canceled)

24. The method of claim 1, which further comprises administering to the subject an additional therapeutic agent.

25. The method of claim 24, wherein the additional therapeutic agent comprises a second inhibitor of a second inhibitory immune checkpoint molecule, an antibiotic, an antipathogen antibody specific for the pathogenic bacterium or pathogenic fungus, or a combination thereof.

26-30. (canceled)

31. The method of claim 7, wherein the subject has received or is about to receive a surgery or an implant.

32-50. (canceled)

51. The method of claim 1, wherein prior to the administration of the inhibitor the subject is determined to have:
(a)(i) an elevated serum titer of CXCL13 compared to a previously measured CXCL13 serum titer for the subject,
(ii) a serum titer of CXCL13 higher than that in healthy control subjects, or
(iii) a serum titer of CXCL13 is at least 50, 55, 60, 65, 70, or 75 pg/ml; and/or
(b)(i) an elevated serum titer of soluble TIM-3 compared to a previously measured TIM-3 serum titer for the subject,
(ii) a serum titer of soluble TIM-3 higher than that in healthy control subjects, or
(iii) a serum titer of soluble TIM-3 is at least 2100, 2200, 2300, 2400, 2500, or 3000 pg/ml; and/or
(c)(i) an elevated serum titer of soluble LAG-3 compared to a previously measured LAG-3 serum titer for the subject,
(ii) a serum titer of soluble LAG-3 higher than that in healthy control subjects, or
(iii) a serum titer of soluble LAG-3 is at least 80000, 85000, 90000, 95000, or 100000 pg/ml; or any combination of (a)-(c).

52-54. (canceled)

55. The method of claim 1, wherein the administered inhibitor further comprises a TIGIT inhibitor.

56-60. (canceled)
